(19) United States
(12) Patent Application Publication
(10) Pub. No.: US 2023/0331649 A1
(43) Pub. Date: Oct. 19, 2023

(54) INHIBITORS OF PROTEIN FUCOSYLATION AND USES THEREOF

(71) Applicant: SIMON FRASER UNIVERSITY, Burnaby (CA)

(72) Inventors: David J. VOCADLO, Burnaby (CA); Robert BRITTON, North Vancouver (CA); Yang WANG, Shandong (CN); Venkata Narasimharao THOTA, Burnaby (CA); Anthony FERS-LIDOU, Burnaby (CA)

(21) Appl. No.: 18/000,423

(22) PCT Filed: Jun. 3, 2021
(86) PCT No.: PCT/IB2021/054844

§ 371 Date: Dec. 1, 2022

Related U.S. Application Data
(60) Provisional application No. 63/034,256, filed on Jun. 3, 2020.

(51) Int. Cl. C07C 35/14; C07F 9/12; C07H 3/02; C12N 5/071

(52) U.S. Cl. CPC C07C 35/14; C07F 9/12; C07H 3/02; C12N 5/0682; C12N 2500/30

(57) ABSTRACT

The present invention relates to inhibitors of protein fucosylation. More specifically, the present invention relates to carbacyclic compounds of Formula (I) useful as inhibitors of protein fucosylation, or for treating a cancer, an autoimmune disease, an infectious disease, an inflammatory disease, or sickle cell disease.

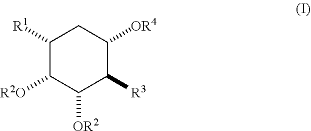

### CROSS-REFERENCE TO RELATED PATENT APPLICATIONS

This application is a U.S. National Stage Application under 35 U.S.C. § 371 of International Patent Application No. PCT/IB2021/054844 filed Jun. 3, 2021, which claims the benefit of priority of U.S. Patent Application No. 63/034,256 filed Jun. 3, 2020, both of which are incorporated by reference in their entireties. The International Application was published on Dec. 9, 2021, as International Publication No. WO/2021/245579A1.

### FIELD OF INVENTION

The present invention relates to inhibitors of protein fucosylation. More specifically, the present invention relates to carbacyclic compounds useful as inhibitors of protein fucosylation.

### BACKGROUND OF THE INVENTION

Fucosylation is a common modification, involving the transfer of a fucose residue from GDP-fucose to an N-glycan, 0-glycan or glycolipid, catalyzed by fucosyltransferases (FUTs). FUTs catalyse the transfer of fucose as follows: FUT1 and 2 catalyse α1,2-linkage; FUT3 to 7 and FUT9 to 11 catalyse α1,3-linkage; FUT3 and 5 catalyse α1,4-linkage; FUT8 catalyses a1,6-linkage (“core fucosylation”); POFUT1 catalyses direct linkage to the serine/threonine residues of EGF-like repeats; and POFUT1 catalyses direct linkage to the serine/threonine residues of thrombospondin repeats. Fucosylation is implicated in a variety of cellular processes.

Antibody-dependent cell-mediated cytotoxicity (ADCC) involves selective targeting and lyses of cells by effector cells of the immune system. ADCC is centrally important for the efficacy of anti-cancer antibodies and is mediated by the binding of the Fc region of an antibody to the Fc Receptor (FcγR) on an effector natural killer (NK) cell and between the antibody and membrane exposed antigens on the cancer cell. Most Food and Drug Administration (FDA) approved monoclonal antibodies are of the IgG1 isotype and incorporate two N-linked complex oligosaccharides in their Fc region. The ADCC of anti-cancer antibodies is attenuated by circulating IgGs that non-specifically compete for binding to FcγR on the effector NK cells. To overcome this limitation, several strategies that focus on altering the N-glycan structure in the Fc region of the antibodies have been explored. Most notably, it was found that the production of fucose-deficient (FD) antibodies improves binding to FcγR and leads to significant increases (˜50 fold) in ADCC. Accordingly, there is interest in developing fucose-deficient (FD)-antibodies. Production of FD-antibodies can be accomplished by small molecule inhibition or genetic knock out of key enzymes involved in fucosylation, for example GDP-mannose dehydratase (GMD), GDP-fucose synthase (GFS) or FUT8. Small molecule inhibitors of one or more of these enzymes offer a distinct advantage in that expensive and time-consuming engineering of cell lines is not required and disruption to existing manufacturing processes is minimal.

Several carbohydrate inhibitors of antibody fucosylation have demonstrated utility as additives in the production of FD antibodies, however, these carbohydrate inhibitors are themselves incorporated into the antibody N-glycan to varying degrees. Such inhibitors may present significant drug consistency risks and may be immunogenic.

### SUMMARY OF THE INVENTION

The present invention relates to inhibitors of protein fucosylation. More specifically, the present invention relates to carbacyclic compounds useful as inhibitors of protein fucosylation.

In one aspect, the present invention provides a method of inhibiting fucosylation of a protein, or fragment or derivative thereof by contacting a eukaryotic cell or a mammal with a compound of Formula (I) or a salt thereof:

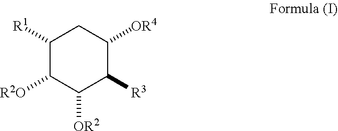

where
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring,
where fucosylation of the protein is reduced by at least 5% in the eukaryotic cell or mammal relative to the amount of fucosylation of the protein in the absence of administration of the compound.

In some embodiments, the protein may be an N-glycan.

In some embodiments, the compound may not be incorporated into the N-glycan.

In some embodiments, the protein may be an antibody.

In some embodiments, R¹ may be CH₃ or CHCH₂, R² may be H, R³ may be OH, and R⁴ may be H or —P(═O)(OR⁵)(OR⁶), where R⁵ and R⁶ may be H.

In some embodiments, the compound may be

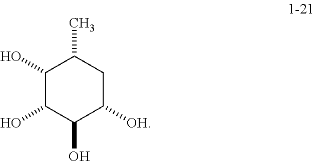

In some embodiments, the mammal may have a cancer, an autoimmune disease, an inflammatory disease, or an infectious disease.

In some embodiments, the method may further include administering a cancer-associated antigen or an antigenic fragment thereof as an immunogen to the mammal having a cancer.

In some embodiments, the mammal may be a human.

In some embodiments, the salt may be a pharmaceutically acceptable salt.

In another aspect, the present invention provides a mammalian cell culture medium comprising an effective amount of a compound of Formula (I) or a salt thereof:

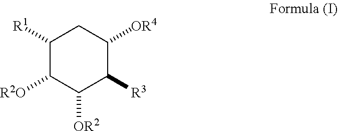

where
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring.

In some embodiments, the medium may be useful for the production of a fucose-deficient protein, or fragment or derivative thereof.

In some embodiments, the effective amount may be an amount of the compound is an amount sufficient to decrease fucose incorporation into a sugar chain of the fucose-deficient protein or fragment or derivative thereof by at least 50%.

In some embodiments, the medium may be a Chinese hamster ovary cell culture medium.

In another aspect, the present invention provides a method of treating a cancer, an autoimmune disease, an infectious disease, an inflammatory disease, or sickle cell disease by administering an effective amount of a compound of Formula (I) or a pharmaceutically acceptable salt thereof:

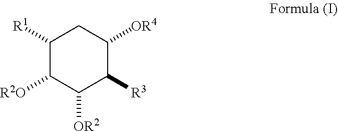

where
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring, to a mammal in need thereof.

In another aspect, the present invention provides a use of a compound of Formula (I) or a pharmaceutically acceptable salt thereof:

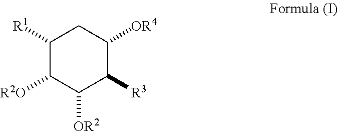

where
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring,
for inhibiting fucosylation of a protein, or fragment or derivative thereof, wherein fucosylation of the protein is reduced by at least 5% in the eukaryotic cell or mammal relative to the amount of fucosylation of the protein in the absence of the compound, or for treating a cancer, an autoimmune disease, an infectious disease, an inflammatory disease, or sickle cell disease.

In another aspect, the present invention provides a compound of Formula (II) or a pharmaceutically acceptable salt thereof:

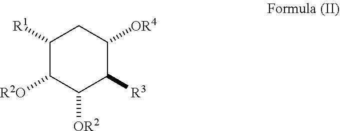

where
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring, wherein when R¹ is CH₃, R² is not H, R³ is not OH, and R⁴ is not H.

In another aspect, the present invention provides a composition comprising the compound of Formula II.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-D show the results of cell-based assays on carbafucose and carbafucose analogues, in accordance with an embodiment of the present invention. (A) 2-deoxy-2-fluoro-L-fucose (2FFuc); (B) carbafucose (1-21); (C) carbafucose analog 2-3 (phosphate); and (D) carbafucose analog 1-42 (alkene).

### DETAILED DESCRIPTION

The present disclosure provides, in part, the production of carbacyclic analogues of sugars and their use in modifying carbohydrate incorporation into proteins, such as antibodies, during production of such proteins. In some embodiments, the present disclosure provides the production and use of carbafucose and analogues of carbafucose that may, for example, be useful in inhibiting the amount of core fucosylation of antibodies and proteins. In some embodiments, the compounds according to the present disclosure reduce protein, for example antibody, fucosylation. In some embodiments, the compounds according to the present disclosure are not incorporated in the protein, for example antibody, N-glycan.

In some embodiments, the present disclosure provides carbafucose and analogues thereof, as well as methods of making the same. By “carbafucose” is meant a compound having the following structure:

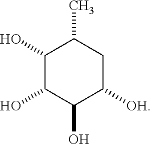

In one aspect, the present disclosure provides a compound of Formula I or a salt thereof:

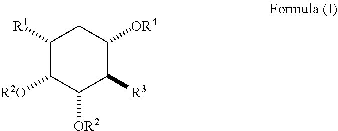

where
R¹ may be optionally substituted C₁-C₁₀ alkyl, optionally substituted C₁-C₁₀ alkenyl, or optionally substituted C₁-C₁₀ alkynyl;
R² may be H or —C(═O)(C₁-C₁₀)alkyl;
R³ may be halo, OH, or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In an alternative aspect, the present disclosure provides a compound of Formula II or a salt thereof:

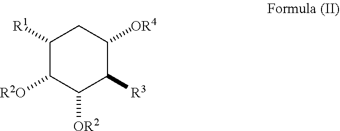

where
R¹ may be optionally substituted C₁-C₁₀ alkyl, optionally substituted C₁-C₁₀ alkenyl, or optionally substituted C₁-C₁₀ alkynyl;
R² may be H or —C(═O)(C₁-C₁₀)alkyl;
R³ may be halo, OH, or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring,
where when R¹ is CH₃, R² is not H, R³ is not OH, and R⁴ is not H.

In some embodiments, R¹ may be C₁-C₁₀ alkyl, C₂-C₁₀alkenyl, C₂-C₁₀ alkynyl, perhalo(C₁-C₁₀)alkyl, or halo(C₁-C₁₀)alkyl.

In some embodiments, R¹ may be C₁-C₆alkyl, C₂-C₆ alkenyl, C₂-C₆ alkynyl, perhalo(C₁-C₆)alkyl, or halo(C₁-C₆)alkyl.

In some embodiments, R¹ may be acyl or ester.

In some embodiments, R¹ may be selected from the group consisting of CH₃, CH₂F, CHF₂, CF₃, CCH, CCCH₃, COCH₃, COCH₂CH₃, CHCH₂, CO₂CH₃, and CH₂Br.

In some embodiments, R¹ may be CH₃.

In some embodiments, R¹ may be CHCH₂.

In some embodiments, R² may be H or —C(═O)(C₁-C₆)alkyl.

In some embodiments, R² may be H.

In some embodiments, R² may be —C(═O)(C₁-C₆)alkyl.

In some embodiments, R³ may be halo, OH, or O—C(═O)(C₁-C₆)alkyl.

In some embodiments, R³ may be OH.

In some embodiments, R³ may be F.

In some embodiments, R³ may be OH or O—C(═O)(C₁-C₆)alkyl.

In some embodiments, R⁴ may be H, O—C(═O)(C₁-C₆)alkyl, or —P(═O)(OR⁵)(OR⁶), where R⁵ and R⁶ may each independently be H, (C₁-C₆)alkyl or (CH₂)₂SC(═O)CH₃.

In some embodiments, R⁴ may be H.

In some embodiments, R⁴ may be —P(═O)(OR⁵)(OR⁶), where R⁵ and R⁶ may each independently be H.

In some embodiments, R⁴ may be —P(═O)(OR⁵)(OR⁶), where R⁵ and R⁶ may both be H.

In some embodiments, R¹ may be CH₃, R² may be H or —C(═O)CH₃, R³ may be OH or O—C(═O)CH₃, and R⁴ may be H or —C(═O)CH₃.

In some embodiments, R¹ may be CH₃, R² may be H, R³ may be OH, and R⁴ may be H.

In some embodiments, R¹ may be CH₃, R² may be H, R³ may be OH, and R⁴ may be —P(═O)(OR⁵)(OR⁶), where R⁵ and R⁶ may be H.

In some embodiments, R¹ may be CH₂F, R² may be H or —C(═O)CH₃, R³ may be OH or O—C(═O)CH₃, and R⁴ may be H or —C(═O)CH₃.

In some embodiments, R¹ may be CHF₂, R² may be H or —C(═O)CH₃, R³ may be OH or O—C(═O)CH₃, and R⁴ may be H or —C(═O)CH₃.

In some embodiments, R¹ may be CF₃, R² may be H or —C(═O)CH₃, R³ may be OH or O—C(═O)CH₃, and R⁴ may be H or —C(═O)CH₃.

In some embodiments, R¹ may be CCH, R² may be H or —C(═O)CH₃, R³ may be OH or O—C(═O)CH₃, and R⁴ may be H or —C(═O)CH₃.

In some embodiments, R¹ may be CCCH₃, R² may be H or —C(═O)CH₃, R³ may be OH or O—C(═O)CH₃, and R⁴ may be H or —C(═O)CH₃.

In some embodiments, R¹ may be COCH₃, R² may be H or —C(═O)CH₃, R³ may be OH or O—C(═O)CH₃, and R⁴ may be H or —C(═O)CH₃.

In some embodiments, R¹ may be COCH₂CH₃, R² may be H or —C(═O)CH₃, R³ may be OH or O—C(═O)CH₃, and R⁴ may be H or —C(═O)CH₃.

In some embodiments, R¹ may be CHCH₂, R² may be H or —C(═O)CH₃, R³ may be H or —C(═O)CH₃, and R⁴ may be H or —C(═O)CH₃.

In some embodiments, R¹ may be CHCH₂, R² may be H, R³ may be OH, and R⁴ may be H.

In some embodiments, R¹ may be CO₂CH₃, R² may be H or —C(═O)CH₃, R³ may be OH or O—C(═O)CH₃, and R⁴ may be H or —C(═O)CH₃.

In some embodiments, R¹ may be CH₂Br, R² may be H or —C(═O)CH₃, R³ may be OH or O—C(═O)CH₃, and R⁴ may be H or —C(═O)CH₃.

In some embodiments, R¹ may be CH₃ or CHCH₂, R² may be H, R³ may be OH, and R⁴ may be H or —P(═O)(OR⁵)(OR⁶), where R⁵ and R⁶ may be H.

In some embodiments, the present disclosure provides a compound of Formula (III) or a salt thereof:

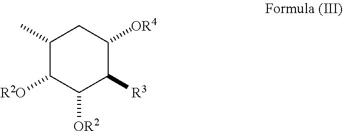

where
R² may be —C(═O)(C₁-C₁₀)alkyl;
R³ may be O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (IV) or a salt thereof:

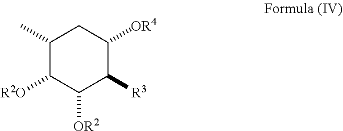

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (V) or a salt thereof:

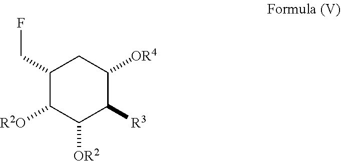

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (VI) or a salt thereof:

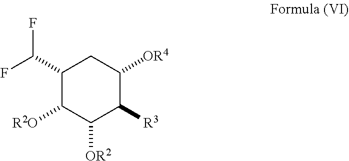

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (VII) or a salt thereof:

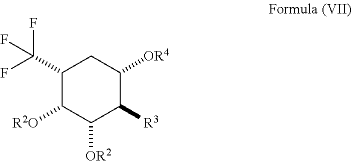

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (VIII) or a salt thereof:

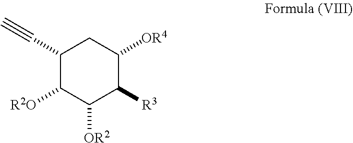

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (IX) or a salt thereof:

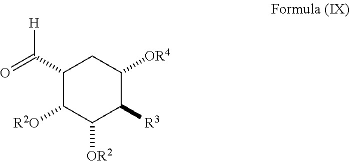

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (X) or a salt thereof:

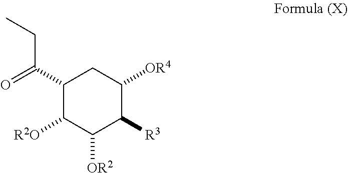

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (XI) or a salt thereof:

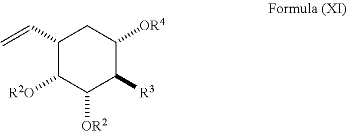

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (XII) or a salt thereof:

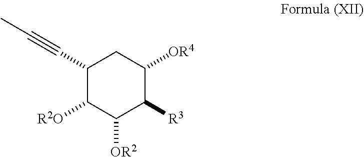

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (XIII) or a salt thereof:

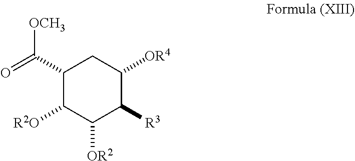

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

In some embodiments, the present disclosure provides a compound of Formula (XIV) or a salt thereof:

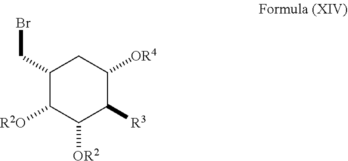

where
R² may be H or —C(═O)(C₁-C₁₀)alkyl;
R³ may be OH or O—C(═O)(C₁-C₁₀)alkyl; and
R⁴ may be H, —C(═O)(C₁-C₆)alkyl, or —P(═O)(OR⁵)(OR⁶), where R⁵ and R⁶ may each independently be H, (C₁-C₆)alkyl, (CH₂)₂SC(═O)CH₃, CH₂OC(═O)OR⁷ or CH₂OC(═O)R⁷ where R⁷ is C₁-C₈ alkyl, or where R⁵ and R⁶ may be connected to form a ring.

“Alkyl” refers to a straight or branched hydrocarbon chain group consisting solely of carbon and hydrogen atoms, containing no unsaturation and including, for example, from one to ten carbon atoms, or any value in between, such as 1, 2, 3, 4, 5, 6, 7, 8, 9 or 10 carbon atoms, and which is attached to the rest of the molecule by a single bond. In some embodiments, alkyl may refer to a straight or branched hydrocarbon chain group consisting solely of carbon and hydrogen atoms, containing no unsaturation and including from one to six carbon atoms, or any value in between, such as 1, 2, 3, 4, 5, or 6 carbon atoms, and which is attached to the rest of the molecule by a single bond. Unless stated otherwise specifically in the specification, the alkyl group may be optionally substituted by one or more substituents as described herein. Unless stated otherwise specifically herein, it is understood that the substitution can occur on any carbon of the alkyl group.

“Alkenyl” refers to a straight or branched hydrocarbon chain group consisting solely of carbon and hydrogen atoms, containing at least one double bond and including, for example, from two to ten carbon atoms, or any value in between, such as 2, 3, 4, 5, 6, 7, 8, 9 or 10 carbon atoms, and which is attached to the rest of the molecule by a single bond. In some embodiments, alkenyl may refer to a straight or branched hydrocarbon chain group consisting solely of carbon and hydrogen atoms, containing at least one double bond and including from two to six carbon atoms, or any value in between, such as 2, 3, 4, 5, or 6 carbon atoms. Unless stated otherwise specifically in the specification, the alkenyl group may be optionally substituted by one or more substituents as described herein. Unless stated otherwise specifically herein, it is understood that the substitution can occur on any carbon of the alkenyl group.

“Alkynyl” refers to a straight or branched hydrocarbon chain group consisting solely of carbon and hydrogen atoms, containing at least one triple bond and including, for example, from two to ten carbon atoms, or any value in between, such as 2, 3, 4, 5, 6, 7, 8, 9 or 10 carbon atoms, and which is attached to the rest of the molecule by a single bond. In some embodiments, alkynyl may refer to a straight or branched hydrocarbon chain group consisting solely of carbon and hydrogen atoms, containing at least one triple bond and including from two to six carbon atoms, or any value in between, such as 2, 3, 4, 5, or 6 carbon atoms. Unless stated otherwise specifically in the specification, the alkynyl group may be optionally substituted by one or more substituents as described herein.

“Acyl” refers to a group of the formula —C(O)Rₐ, where Rₐ is a C₁₋₁₀ alkyl or a C₁₋₆ alkyl group as described herein. The alkyl group may be optionally substituted as described herein.

“Optional” or “optionally” means that the subsequently described event of circumstances may or may not occur, and that the description includes instances where the event or circumstance occurs one or more times and instances in which it does not. For example, “optionally substituted alkyl” means that the alkyl group may or may not be substituted and that the description includes both substituted alkyl groups and alkyl groups having no substitution, and that the alkyl groups may be substituted one or more times. Examples of suitable optional substituents include, without limitation, halo, oxo, or ester. In some embodiments, the oxo group may be at the C₁ position of R₁ of Formula I, as described herein. In some embodiments, the ester group may be at the C₁ position of R₁ of Formula I, as described herein.

“Halo” refers to bromo, chloro, fluoro, iodo, etc. In some embodiments, suitable halogens include fluorine or bromine.

The terms “inhibit,” “inhibiting,” or “inhibition of” means to reduce by a measurable amount, or to prevent entirely.

In some embodiments, the compounds according to the present disclosure may be perhalo compounds.

In some embodiments, in the compounds according to the present disclosure, R⁵ and R⁶ may be connected to form a ring. In such embodiments, “ring” refers to a monocyclic phosphate ester having up to 7 members that may be saturated or monounsaturated. In some embodiments, R⁵ and R⁶ may be connected to form an aryl phosphate ester.

In some embodiments, the salt of the compound according to the present disclosure may be any suitable salt such as, without limitation, a sodium salt, potassium salt, an ammonium salt etc. In some embodiments, the salt of the compound according to the present disclosure may be a pharmaceutically acceptable salt.

Production of FD-Proteins

In some embodiments, the compounds according to the present disclosure may be used in the production of “fucose-deficient” or “FD”-proteins or antibodies using standard techniques as, for example, described herein or in U.S. Pat. No. 9,816,069, granted Nov. 14, 2017; PCT publication WO/2012/019165, published Feb. 9, 2012; or known in the art. Accordingly, the present disclosure provides, in part, methods for inhibiting fucosylation of a protein, or fragment or derivative thereof, by contacting a eukaryotic cell or a mammal with a compound as described herein, such as a compound of Formula I, II, III, IV, V, VI, VII, VIII, IX, X, XI, XII, XIII, or XIV, e.g., carbafucose, or a salt thereof, using standard techniques as described herein or known in the art. The methods may be conducted in vitro or in vivo.

In general, fucosylation refers to the transfer of fucose from GDP to a glycan by α(1,2)-, α(1,3)-, α(1,4)- and/or α(1,6)-linkage. Core fucosylation refers to the transfer of fucose from GDP to the innermost N-acetylglucosamine (GIcNAc) residue (the reducing terminal) of an N-linked glycan of a protein, such as an antibody, by a1,6-linkage by alpha-1,6-fucosyl transferase 8 (FUT8).

By a “fucose-deficient” or “FD” protein is meant a polypeptide, such as an antibody, with reduced fucosylation compared to a polypeptide, such as an antibody, produced in the absence of a carbacyclic compound, for example a compound as described herein, such as a compound of Formula I, II, III, IV, V, VI, VII, VIII, IX, X, XI, XII, XIII, or XIV, e.g., carbafucose. The reduction in fucosylation can be at least about 5% to 100%, or any value in between, such as at least about 5%, 10%, 15%, 20%, 25%, 30%, 35%, 40%, 45%, 50%, 55%, 60%, 65%, 70%, 75%, 80%, 85%, 90%, or 95%, or 99%. In some embodiments, the reduction in fucosylation can be at least about 50% to 100%, or any value in between, such as at least about 50%, 55%, 60%, 65%, 70%, 75%, 80%, 85%, 90%, or 95%, or 99%. In some embodiments, the reduction in fucosylation can be a reduction in “core fucosylation.”

In some embodiments, only a minor amount of fucose may be incorporated into the sugar chain(s) (e.g., a glycan, such as an N-glycan) after administering a carbacyclic compound as described herein, e.g., carbafucose. For example, in various embodiments, less than about 60%, less than about 50%, less than about 40%, less than about 30%, less than about 20%, less than about 15%, less than about 10%, less than about 5%, or less than about 1% of the antibodies in the serum of the animal (e.g., a mammal, such as a human) are fucosylated, as compared to cell or an animal not receiving the carbacyclic compound as described herein, e.g., carbafucose. In some embodiments, substantially none (i.e., less than 0.5%) of the antibodies in the serum of the animal are not fucosylated, as compared to an animal not receiving the carbacyclic compound as described herein, e.g., carbafucose.

A protein or antibody for use as described herein can be made using recombinant techniques. Recombinant expression of a protein or an antibody, or a fragment or derivative thereof that binds to a target antigen, typically involves construction of an expression vector containing a nucleic acid that encodes the antibody or derivative thereof. Once a nucleic acid encoding such a protein has been obtained, the vector for the production of the protein molecule may be produced by recombinant DNA technology using techniques well known in the art. Standard techniques such as those described in Sambrook and Russell, Molecular Cloning: A Laboratory Manual (Cold Spring Harbor Laboratory Press, Cold Spring Harbor, N.Y., 3rd ed., 2001); Sambrook et al., Molecular Cloning: A Laboratory Manual (Cold Spring Harbor Laboratory Press, Cold Spring Harbor, N.Y., 2nd ed., 1989); Ausubel et al., Short Protocols in Molecular Biology (John Wiley & Sons, New York, 4th ed., 1999); and Glick & Pasternak, Molecular Biotechnology: Principles and Applications of Recombinant DNA (ASM Press, Washington, D.C., 2nd ed., 1998) can be used for recombinant nucleic acid methods, nucleic acid synthesis, cell culture, transgene incorporation, and recombinant protein expression.

Accordingly, in some embodiments, antibodies or fragments or derivatives thereof can be produced using recombinant expression techniques from, for example, hybridomas, myelomas or other suitable cells, including mammalian cells.

For example, for recombinant expression of an antibody or a fragment or derivative thereof, an expression vector can encode a heavy or light chain thereof, or a heavy or light chain variable domain, operably linked to a promoter. An expression vector can include, for example, the nucleotide sequence encoding the constant region of the antibody molecule (see, for example, PCT publications WO 86/05807 or WO 89/01036; or U.S. Pat. No. 5,122,464), and the variable domain of the antibody can be cloned into such a vector for expression of the entire heavy or light chain. The expression vector can be transferred to a host cell by techniques known in the art, and the transfected cells can then cultured by techniques known in the art in the presence of a carbacyclic compound as described herein, e.g., carbafucose, to produce the antibody. Typically, for the expression of double-chained antibodies, vectors encoding both the heavy and light chains can be co-expressed in the host cell for expression of the entire immunoglobulin molecule.

Any suitable mammalian cell or cell line can be used to express an antibody or fragment or derivative thereof. For example, mammalian cells such as Chinese hamster ovary cells (CHO) (e.g., DG44, Dxb11, CHO-K, CHO-K1 and CHO-S) can be used. In some embodiments, human cell lines can be used. Suitable myeloma cell lines include without limitation SP2/0 and IR983F and human myeloma cell lines such as Namalwa. Other suitable cells include without limitation human embryonic kidney cells (e.g., HEK293), monkey kidney cells (e.g., COS), human epithelial cells (e.g., HeLa), PERC6, Wil-2, Jurkat, Vero, Molt-4, BHK, and K6H6. Other suitable host cells include without limitation YB2/0 cells.

In some embodiments, the host cells can be from a hybridoma.

In some embodiments, the host cells do not contain a fucose transporter gene knockout. In some embodiments, the host cells do not contain a fucosyltransferase (e.g., FUT8) gene knockout. In some embodiments, the host cells do not contain a knock-in of a GnTIII encoding nucleic acid. In some embodiments, the host cells do not contain a knock-in of a golgi alpha mannosidase II encoding nucleic acid.

A variety of mammalian host-expression vector systems can be utilized to express an antibody or fragment or derivative thereof. For example, mammalian cells such as Chinese hamster ovary cells (CHO) (such as DG44, Dxb11, CHO-K1 and CHO-S) in conjunction with a vector such as the major intermediate early gene promoter element from human cytomegalovirus or the Chinese hamster ovary EF-1α promoter, is an effective expression system for the production of antibodies and derivatives thereof (see, for example, Foecking et al., 1986, Gene 45:101; Cockett et al., 1990, Bio/Technology 8:2; Allison, U.S. Pat. No. 5,888,809). In some embodiments, suitable cell lines may be Chinese Hamster Ovary (CHO) cell lines or CHO-derived cell lines.

The cell lines can be cultured in a suitable culture medium. Suitable culture media include those containing, for example, salts, carbon source (such as sugars), nitrogen source, amino acids, trace elements, antibiotics, selection agents, and the like, as required for growth. For example, commercially available media such as Ham's F10 (Sigma), Minimal Essential Medium (MEM, Sigma), RPMI-1640 (Sigma), Dulbecco's Modified Eagle's Medium ((DMEM, Sigma), PowerCHO™ cell culture media (Lonza Group Ltd.) Hybridoma Serum-Free Medium (HSFM) (GIBCO), as appropriate, can be suitable for culturing the host cells. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements can also be included at appropriate concentrations that would be known to those skilled in the art. The culture conditions, such as temperature, pH, and the like, can be those previously used with the host cell selected for expression, as known in the art. In some embodiments, the culture medium is not supplemented with fucose. In some embodiments, the culture medium can be serum-free. In some embodiments, the culture medium can be animal-derived protein free.

In some embodiments, an effective amount of a compound as described herein, such as a compound of Formula I, II, III, IV, V, VI, VII, VIII, IX, X, XI, XII, XIII, or XIV, e.g., carbafucose, or a salt thereof, may added to a culture medium. In alternative embodiments, the culture medium can include an effective amount of a compound as described herein, such as a compound of Formula I, II, III, IV, V, VI, VII, VIII, IX, X, XI, XII, XIII, or XIV, e.g., carbafucose, or a salt thereof. Accordingly, in some embodiments, a compound as described herein, such as a compound of Formula I, II, III, IV, V, VI, VII, VIII, IX, X, XI, XII, XIII, or XIV, e.g., carbafucose, or a salt thereof, may be provided in a kit together with instructions for use in inhibiting fucosylation of a protein, such as an antibody, or fragment or derivative thereof. The kit may further include, without limitation, a cell culture medium, cell line, etc.

To produce a FD-protein or FD-antibody, or fragment or derivative thereof, an effective amount of a carbacyclic compound, for example a compound as described herein, such as a compound of Formula I, II, III, IV, V, VI, VII, VIII, IX, X, XI, XII, XIII, or XIV, e.g., carbafucose, can be added to the culture medium, or the cell culture medium can include an effective amount of the carbacyclic compound. In this context, an “effective amount” refers to an amount of the carbacyclic compound as described herein that is sufficient to reduce fucose incorporation into a sugar chain of the protein or antibody, or fragment or derivative thereof, by at least about 5% to 100%, or any value in between, such as at least about 5%, 10%, 15%, 20%, 25%, 30%, 35%, 40%, 45%, 50%, 55%, 60%, 65%, 70%, 75%, 80%, 85%, 90%, or 95%, or 99%. In some embodiments, the effective amount of the carbacyclic compound as described herein can be sufficient to reduce fucose incorporation into a sugar chain of the protein or antibody, or fragment or derivative thereof, by at least about 50%.

The cells expressing the protein or antibody, or fragment or derivative thereof, can be cultured by growing the host cell in any suitable volume of culture medium supplemented with the carbacyclic compound as described herein. The cells may be cultured in any suitable culture system and according to any method known in the art, including T-flasks, spinner and shaker flasks, WaveBag™ bags, roller bottles, bioreactors and stirred-tank bioreactors. Anchorage-dependent cells can also be cultivated on microcarrier, e.g., polymeric spheres, that are maintained in suspension in stirred-tank bioreactors. Alternatively, cells can be grown in single-cell suspension. Culture medium may be added in a batch process, e.g., where culture medium is added once to the cells in a single batch, or in a fed batch process in which small batches of culture medium are periodically added. Medium can be harvested at the end of culture or several times during culture. Continuously perfused production processes are also known in the art and involve continuous feeding of fresh medium into the culture, while the same volume is continuously withdrawn from the reactor. Perfused cultures generally achieve higher cell densities than batch cultures and can be maintained for weeks or months with repeated harvests.

For cells grown in batch culture, the volume of culture medium can be at least 750 mL, 1 liter, 2 liters, 3 liters, 4 liters, 5 liters, 10 liters, 15 liters, 20 liters or more. For industrial applications, the volume of the culture medium can be at least 100 liters, at least 200 liters, at least 250 liters, at least 500 liters, at least 750 liters, at least 1000 liters, at least 2000 liters, at least 5000 liters or at least 10,000 liters. A carbacyclic compound as described herein, e.g., carbafucose, can be added to the seed train, to the initial batch culture medium, after a rapid growth phase, or continuously with culture medium (e.g., during continuous feeding). For example, a carbacyclic compound as described herein, e.g., carbafucose, can be added to an early seed train or feedstock at a 10× or 100× concentration, such that subsequent additions of culture media change the concentration of the carbacyclic compound to a level that is still effective. Alternatively, a carbacyclic compound as described herein, e.g., carbafucose, can be added directly to the culture medium, obviating the need for dilution. In some embodiments, the carbacyclic compound, for example a compound as described herein, such as a compound of Formula I, II, III, IV, V, VI, VII, VIII, IX, X, XI, XII, XIII, or XIV, e.g., carbafucose, can be added relatively early in the cell culturing process and an effective concentration can be maintained throughout the culturing process to optimize production of the desired protein or antibody, or fragment or derivative thereof.

In some embodiments, proteins or antibodies, or fragments or derivatives thereof, produced as described herein exhibit a reduction of at least 10% fucosylation, as compared with antibodies or antibody derivatives produced from the host cells cultured in the absence of a carbacyclic compound as described herein, e.g., carbafucose. In some embodiments, proteins or antibodies, or fragments or derivatives thereof, produced by the instant methods comprise at least as described herein exhibit a reduction of at least 50% fucosylation, as compared with antibodies or antibody derivatives produced from the host cells cultured in the absence of a carbacyclic compound as described herein, e.g., carbafucose.

The amount of the carbacyclic compound as described herein (for example, any of Formulae I, II, III, IV, V, VI, VII, VIII, IX, X, XI, XII, XIII, or XIV, e.g., carbafucose) that is effective can be determined by standard cell culture methodologies. For example, cell culture assays can be conducted to determine optimal dosing ranges. The precise amount to be used can depend on the time of administration, the host cell line, the cell density, etc. Effective doses may be extrapolated from dose-response curves derived from in vitro model or test systems.

In some embodiments, a carbacyclic compound as described herein, e.g., carbafucose, can be present in the culture medium at a concentration of 10 nM to 50 mM. In some embodiments, a carbacyclic compound as described herein, e.g., carbafucose, can be present in the culture medium at a concentration of 10 nM to 10 mM. In some embodiments, the carbacyclic compound as described herein, e.g., carbafucose, can be present in the culture medium at a concentration of 100 nM to 5 mM. In some embodiments, the carbacyclic compound as described herein, e.g., carbafucose, can be present in the culture medium at a concentration of 100 nM to 3 mM. In some embodiments, the carbacyclic compound as described herein, e.g., carbafucose, can be present in the culture medium at a concentration of 100 nM to 2 mM. In some embodiments, the carbacyclic compound as described herein, e.g., carbafucose, can be present in the culture medium at a concentration of 100 nM to 1 mM. In some embodiments, the carbacyclic compound as described herein, e.g., carbafucose, can be present in the culture medium at a concentration of 1 μM to 1 mM. In some embodiments, the carbacyclic compound as described herein, e.g., carbafucose, can be present in the culture medium at a concentration of 10 nM to 1 mM. In some embodiments, the carbacyclic compound as described herein, e.g., carbafucose, can be is present in the culture medium at a concentration of 10 nM to 500 μM. In some embodiments, the carbacyclic compound as described herein can be present in the culture medium at a concentration of 1 μM to 500 μM. In some embodiments, the carbacyclic compound as described herein can be present, e.g., carbafucose, in the culture medium at a concentration of 1 μM to 250 μM. In some embodiments, the carbacyclic compound as described herein, e.g., carbafucose, can be present in the culture medium at a concentration of 10 μM to 100 μM. In some embodiments, the carbacyclic compound as described herein, e.g., carbafucose, can be soluble in the culture medium (at the appropriate temperature for host cell maintenance/growth) at a concentration of at least 100 nM.

The content (e.g., the ratio) of sugar chains in which fucose is not bound to N-acetylglucosamine in the reducing end of the sugar chain versus sugar chains in which fucose is bound to N-acetylglucosamine in the reducing end of the sugar chain can be determined, for example, as described in U.S. Patent Application Publication No. 2004-0110282; by hydrazinolysis or enzyme digestion (see, e.g., Biochemical Experimentation Methods 23: Method for Studying Glycoprotein Sugar Chain (Japan Scientific Societies Press), edited by Reiko Takahashi (1989)), fluorescence labeling or radioisotope labeling of the released sugar chain and then separating the labeled sugar chain by chromatography or any other suitable technique. The compositions of the released sugar chains can be determined by analyzing the chains by the HPAEC-PAD method (see, e.g., J. Liq Chromatogr. 6:1557 (1983)) or any other suitable technique.

In some embodiments, the antibodies, or fragments or derivatives thereof, produced as described herein can have higher effector function (e.g., ADCC activity) than the antibodies, or fragments or derivatives thereof, produced in the absence of a carbacyclic compound as described herein. The effector function activity may be modulated by altering the concentration of the carbacyclic compound as described herein in the culture medium and/or the duration of exposure to the carbacyclic compound as described herein. ADCC activity may be measured using assays known in the art and in exemplary embodiments increases by at least 10%, 20%, 30%, 40%, 50%, 60%, 70%, 80%, 90%, 2-fold, 3-fold, 4-fold, 5-fold, 6-fold, 7-fold, 8-fold, 9-fold, 10-fold, 15-fold or 20-fold, as compared to the fucosylated parent antibody. The cytotoxic activity against an antigen-positive cultured cell line can be evaluated by measuring effector function (e.g., as described in Cancer Immunol. Immunother. 36:373 (1993)).

Proteins or antibodies, or fragments or derivatives thereof, can be purified using, for example, hydroxyapatite chromatography, gel electrophoresis, dialysis, and affinity chromatography. In some embodiments, proteins or antibodies, or fragments or derivatives thereof, can be purified using affinity chromatography.

In some embodiments, Protein A can be used to purify antibodies, or fragments or derivatives thereof, that are based on human IgGI, 2, or 4 heavy chains. The suitability of protein A as an affinity ligand can depend on the species and isotype of any immunoglobulin Fc domain that is present in the antibody, or fragment or derivative thereof.

In some embodiments, Protein G can be used for mouse isotypes or for some human antibodies, or fragments or derivatives thereof. The matrix to which the affinity ligand is attached can be agarose or any other suitable matrix. Mechanically stable matrices such as controlled pore glass or poly(styrenedivinyl)benzene allow for faster flow rates and shorter processing times than can be achieved with agarose. Where the antibody, or fragment or derivative thereof, includes a C_{H3} domain, the Bakerbond ABX™ resin (J. T. Baker, Phillipsburg, N.J.) can be useful for purification. Other techniques for protein purification such as fractionation on an ion-exchange column (cationic or anionic exchange), ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody, or fragment or derivative thereof, to be recovered.

Following any purification step(s), the mixture including the antibody of interest, or fragment or derivative thereof, and contaminants can be subjected to low pH hydrophobic interaction chromatography (e.g., using an elution buffer at a pH between about 2.5-4.5, preferably performed at low salt concentrations (e.g., from about 0-0.25 M salt)).

In general, it is to be understood that any suitable procedure, described herein or known in the art, in combination with a carbacyclic compound as described herein, e.g., carbafucose, can be used in the production of FD-proteins and/or FD-antibodies, or fragments or derivatives thereof.

Antibodies, or fragments or derivatives thereof, that can be produced as described herein can be monoclonal, chimeric, humanized (including veneered), or human antibodies. Suitable antibodies also include antibody fragments, such as single chain antibodies, or the like that have a Fc region or domain having a complex N-glycoside-linked sugar chain (e.g., a human IgG1 Fc region or domain). The Fc region or domain can include an Fcgamma receptor binding site. The antibodies can be human or humanized. In some embodiments, the antibodies can be rodent (e.g., mouse and rat), donkey, sheep, rabbit, goat, guinea pig, camelid, horse, or chicken.

The antibodies, or fragments or derivatives thereof, can be mono-specific, bi-specific, tri-specific, or of greater multi-specificity. Multi-specific antibodies maybe specific for different epitopes of different target antigens or may be specific for different epitopes on the same target antigen. (See, e.g., WO 93/17715; WO 92/08802; WO 91/00360; WO 92/05793; Tutt et al., 1991, J. Immunol. 147:60-69; U.S. Pat. Nos. 4,474,893; 4,714,681; 4,925,648; 5,573,920; and U.S. Pat. No. 5,601,819; Kostelny et al., 1992, J. Immunol. 148:1547-1553.)

The antibodies, or fragments or derivatives thereof, can also be described in terms of their specificity of binding to a target antigen.

By “antibody” is meant a polypeptide belonging to the immunoglobulin family or fragment thereof, such as an antigen-binding fragment thereof, as well as such conservative substitutions of such polypeptides and fragments, as discussed further herein. Antibodies are generally described in, for example, Harlow & Lane, Antibodies: A Laboratory Manual (Cold Spring Harbor Laboratory Press, 1988). Unless otherwise apparent from the context, reference to an antibody also includes antibody derivatives as described herein.

“Native antibodies” or “native immunoglobulins” are generally heterotetrameric glycoproteins of about 150,000 daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. The “variable domain” differ extensively in sequence and are involved in the binding and specificity of an antibody for its antigen. The variability is concentrated in three segments, termed “hypervariable regions” in both the light chain and the heavy chain variable domains while the less variable sequences are termed the “framework region” or “FR.” The constant domains are not involved directly in antigen binding, instead exhibiting various effector functions.

The term “Fc region” refers to the constant region of an antibody, e.g., a C_{H}1-hinge-C_{H}2-C_{H}3 domain, optionally having a CH4 domain, or a conservatively substituted derivative of such an Fc region.

The term “Fc domain” refers to the constant region domain of an antibody, e.g., a C_{H}1, hinge, C_{H}2, C_{H}3 or C_{H}4 domain, or a conservatively substituted derivative of such an Fc domain.

The terms “antigen-binding portion,” “antigen-binding fragment,” or “antigen-binding domain,” “antibody fragment” or a “functional fragment of an antibody” refer to an antibody fragment that retains the ability to specifically bind to an antigen, (see generally, Holliger et al., Nature Biotech. 23 (9) 1126-1129 (2005)). Examples of antibody fragments include, without limitation, Fab fragments, monovalent fragments consisting of V_{L}, V_{H}, C_{L} and C_{H} domains; F(ab′)₂ fragments; bivalent fragments including two Fab fragments linked by a disulfide bridge at the hinge region; Fd fragments consisting of the V_{H} and C_{H} domains; Fv fragments consisting of the V_{L} and V_{H} domains of a single arm of an antibody, (v) dAb fragments consisting of a V_{H} domain; single chain Fv (scFv) molecules consisting of synthetically linked V_{L} and V_{H} domains; diabodies consisting of V_{L} and V_{H} domains synthetically linked synthetically linked a short linker that necessitates pairing with the complementary domains of a different antibody chain, thus creating two antigen-binding sites; isolated complementarity determining regions (CDRs), etc. In general, antigen-binding fragments include complex N-glycoside-linked sugar chain(s).

In some embodiments, the antibody may be a monoclonal antibody. The term “monoclonal antibody” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology.

In some embodiments, the antibody may be a chimeric antibody. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. (See, e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397.)

In some embodiments, the antibody can be a humanized antibody, including a veneered antibody. Humanized antibodies are antibody molecules that bind the desired antigen and have one or more complementarity determining regions (CDRs) from a non-human species, and framework and constant regions from a human immunoglobulin molecule. Often, framework residues in the human framework regions will be substituted with the corresponding residue from the CDR donor antibody to alter, or preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riecbmann et al., 1988, Nature 332:323.) Antibodies can be humanized using a variety of techniques known in the art such as CDR-grafting (EP 0 239 400; WO 91/09967; U.S. Pat. Nos. 5,225,539; 5,530,101; and 5,585,089), veneering or resurfacing (EP 0 592 106; EP 0 519 596; Padlan, 1991, Molecular Immunology, 28 (4/5):489-498; Studnicka et al., 1994, Protein Engineering 7(6):805-814; Roguska et al., 1994, Proc. Natl. Acad. Sci. USA 91:969-973), and chain shuffling (U.S. Pat. No. 5,565,332.

In some embodiments, the antibody can be a human antibody. Human antibodies can be made by a variety of methods known in the art such as phage display methods using antibody libraries derived from human immunoglobulin sequences. See e.g., U.S. Pat. Nos. 4,444,887 and 4,716,111; WO 98/46645, WO 98/50433, WO 98/24893, WO 98/16654, WO 96/34096, WO 96/33735, and WO 91/10741. In addition, a human antibody recognizing a selected epitope can be generated using a technique referred to as “guided selection,” in which a selected non-human monoclonal antibody, e.g., a mouse antibody, is used to guide the selection of a completely human antibody recognizing the same epitope (see, e.g., Jespers et al., 1994, Biotechnology 12:899-903). Human antibodies can also be produced using transgenic mice that express human immunoglobulin genes. Monoclonal antibodies directed against the antigen can be obtained from the immunized, transgenic mice using conventional hybridoma technology. For an overview of the technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598, 877; U.S. Pat. Nos. 5,413,923; 5,625,126; 5,633,425; 5,569,825; 5,661,016; 5,545,806; 5,814,318; 5,885,793; 5,916,771; and 5,939,598.

Examples of antibodies include HERCEPTIN® (trastuzumab; Genentech), RITUXAN® (rituximab; Genentech), lintuzumab (Seattle Genetics, Inc.), Palivizumab (Medimmune), Alemtuzumab (BTG) and Epratuzumab (Immunomedics).

An “antibody derivative” refers to an antibody, or fragment thereof, that is modified by covalent attachment of a heterologous molecule such as by attachment of a heterologous polypeptide (e.g., a ligand binding domain of heterologous protein), or by glycosylation (other than fucosylation), deglycosylation (other than defucosylation), acetylation, phosphorylation or other modification not normally associated with the antibody or fragment thereof.

Examples of antibody derivatives include without limitation binding domain-Ig fusions, wherein the binding domain may be, for example, a ligand, an extracellular domain of a receptor, a peptide, a non-naturally occurring peptide or the like. Exemplary fusions with immunoglobulin or Fc regions include without limitation: etanercept which is a fusion protein of sTNFRII with the Fc region (U.S. Pat. No. 5,605,690), alefacept which is a fusion protein of LFA-3 expressed on antigen presenting cells with the Fc region (U.S. Pat. No. 5,914,111), a fusion protein of Cytotoxic T Lymphocyte-associated antigen-4 (CTLA-4) with the Fc region (J. Exp. Med. 181:1869 (1995)), a fusion protein of interleukin 15 with the Fc region (J. Immunol. 160:5742 (1998)), a fusion protein of factor VII with the Fc region (Proc. Natl. Acad. Sci. USA 98: 12180 (2001)), a fusion protein of interleukin 10 with the Fc region (J. Immunol. 154: 5590 (1995)), a fusion protein of interleukin 2 with the Fc region (J. Immunol. 146: 915 (1991)), a fusion protein of CD40 with the Fc region (Surgery 132:149 (2002)), a fusion protein of Flt-3 (fms-like tyrosine kinase) with the antibody Fc region (Acta. Haemato. 95:218 (1996)), a fusion protein of OX40 with the antibody Fc region (J. Leu. Biol. 72:522 (2002)), and fusion proteins with other CD molecules (e.g., CD2, CD30 (TNFRSF8), CD95 (Fas), CD106 (VCAM-1), CD137), adhesion molecules (e.g., ALCAM (activated leukocyte cell adhesion molecule), cadherins, ICAM (intercellular adhesion molecule)-1, ICAM-2, ICAM-3) cytokine receptors (e.g., interleukin-4R, interleukin-5R, interleukin-6R, interleukin-9R, interleukin-10R, interleukin-12R, interleukin-13Ralpha1, interleukin-13Ralpha2, interleukin-15R, interleukin-21 Ralpha), chemokines, cell death-inducing signal molecules (e.g., B7-H1, DR6 (Death receptor 6), PD-1 (Programmed death-1), TRAIL R1), costimulating molecules (e.g., B7-1, B7-2, B7-H2, ICOS (inducible co-stimulator)), growth factors (e.g., ErbB2, ErbB3, ErbB4, HGFR), differentiation-inducing factors (e.g., B7-H3), activating factors (e.g., NKG2D), signal transfer molecules (e.g., gp130), BCMA, and TACI.

An “antigen” is a molecule to which an antibody, or fragment or derivative thereof, specifically binds.

The terms “specific binding” or “specifically binds” mean that the antibody, or fragment or derivative thereof, will bind, in a highly selective manner, with its corresponding target antigen and not with the multitude of other antigens. For example, the antibody, or fragment or derivative thereof, can bind with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

Antibodies can be assayed for specific binding to a target antigen by conventional methods, such as for example, competitive and non-competitive immunoassay systems using techniques such as Western blots, radioimmunoassays, ELISA (enzyme linked immunosorbent assay), “sandwich” immunoassays, immunoprecipitation assays, immunoradiometric assays, fluorescent immunoassays, and protein A immunoassays. (See, e.g., Ausubel et al., eds., Short Protocols in Molecular Biology (John Wiley & Sons, Inc., New York, 4th ed. 1999); Harlow & Lane, Using Antibodies: A Laboratory Manual (Cold Spring Harbor Laboratory Press, Cold Spring Harbor, N.Y., 1999.) Further, the binding affinity of an antibody to a target antigen and the off-rate of an antibody-antigen interaction can be determined by surface plasmon resonance, competition FACS using labeled antibodies or other competitive binding assays. One example of a competitive binding assay is a radioimmunoassay comprising the incubation of labeled antigen (e.g., ³H or ¹²⁵I) with the antibody of interest in the presence of increasing amounts of unlabeled antibody, and the detection of the antibody bound to the labeled antigen. The affinity of the antibody and the binding off-rates can then be determined from the data by Scatchard plot analysis. Competition with a second antibody can also be determined using radioimmunoassays. In this case, the antigen is incubated with the antibody of interest conjugated to a labeled compound (e.g., ³H or ¹²⁵I) in the presence of increasing amounts of an unlabeled second antibody. Alternatively, the binding affinity of an antibody and the on- and off-rates of an antibody-antigen interaction can be determined by surface plasmon resonance.

In some embodiments, an antibody, or fragment or derivative thereof, specifically binds to CD19, CD20, CD21, CD22, CD30, CD33, CD38, CD40, CD70, CD133, CD138, or CD276. In other embodiments, the antibody, or fragment or derivative thereof, specifically binds to BMPR1B, LAT1 (SLC7A5), STEAP1, MUC16, megakaryocyte potentiating factor (MPF), Napi3b, Sema 5b, PSCA hlg, ETBR (Endothelin type B receptor), STEAP2, TrpM4, CRIPTO, CD21, CD79a, CD79b, FcRH2, HER2, HER3, HER4, NCA, MDP, IL20R.alpha., Brevican, Ephb2R, ASLG659, PSCA, PSMA, GEDA, BAFF-R, CXCR5, HLA-DOB, P2X5, CD72, LY64, FCRH1, or IRTA2.

Antibodies can be made from antigen-containing fragments of the target antigen by standard procedures according to the type of antibody (see, e.g., Kohler, et al., Nature, 256:495, (1975); Harlow & Lane, Antibodies, A Laboratory Manual (C.S.H.P., NY, 1988); Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes). As an example, monoclonal antibodies can be prepared using a wide variety of techniques including, e.g., the use of hybridoma, recombinant, and phage display technologies, or a combination thereof. Hybridoma techniques are generally discussed in, e.g., Harlow et al., supra, and Hammerling, et al., In Monoclonal Antibodies and T-Cell Hybridomas, pp. 563-681 (Elsevier, N.Y., 1981). Examples of phage display methods that can be used to make antibodies include, e.g., those disclosed in Briinnan et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01 134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Examples of techniques that can be used to produce single-chain Fvs and antibodies include those described in U.S. Pat. Nos. 4,946,778 and 5,258,498; Huston et al., 1991, Methods in Enzymology 203:46-88; Shu et al., 1993, Proc. Natl. Acad. Sci. USA 90:7995-7999; and Skerra et al., 1988, Science 240:1038-1040.

Direct Administration

In some embodiments, a carbacyclic compound as described herein, e.g., carbafucose, can be administered to a subject, e.g, a mammal, such as a human, to reduce fucosylation of a protein. Accordingly, the present disclosure provides, in part, methods for reducing or inhibiting fucosylation of a protein, or fragment or derivative thereof, by administering a compound as described herein, such as a compound of Formula I, II, III, IV, V, VI, VII, VIII, IX, X, XI, XII, XIII, or XIV, e.g., carbafucose, or a pharmaceutically acceptable salt thereof, to subject e.g, a mammal, such as a human.

As used herein, a subject may be a mammal, such as a human, non-human primate, rat, mouse, cow, horse, pig, sheep, goat, dog, cat, etc. The subject may be a clinical patient, a clinical trial volunteer, an experimental animal, etc.

In some embodiments, only a minor amount of fucose is incorporated into the sugar chain(s) (e.g., an N-glycan or complex N-glycoside-linked sugar chain) after administering a carbacyclic compound as described herein, e.g., carbafucose. For example, in various embodiments, less than about 60%, less than about 50%, less than about 40%, less than about 30%, less than about 20%, less than about 15%, less than about 10%, less than about 5%, or less than about 1% of the proteins in the animal (e.g., a mammal, such as a human) are core fucosylated, as compared to an animal not receiving the carbacyclic compound as described herein, e.g., carbafucose. In some embodiments, substantially none (i.e., less than 0.5%) of the antibodies in the serum of the animal are not core fucosylated, as compared to an animal not receiving the carbacyclic compound as described herein, e.g., carbafucose.

In some embodiments, protein fucosylation is reduced by about 60%, by about 50%, by about 40%, by about 30%, by about 20%, by about 15%, by about 10%, by about 5%, or by about 1% for cell surface proteins in the animal (e.g., a mammal, such as a human) are fucosylated, as compared to an animal not receiving the carbacyclic compound as described herein, e.g., carbafucose. In some embodiments, protein fucosylation via α(1,2)-linkage is reduced by about 60%, by about 50%, by about 40%, by about 30%, by about 20%, by about 15%, by about 10%, by about 5%, or by about 1% for cell surface proteins in the animal (e.g., a mammal, such as a human) are fucosylated, as compared to an animal not receiving the carbacyclic compound as described herein, e.g., carbafucose.

In some embodiments, protein fucosylation via α(1,3)-linkage is reduced by about 60%, by about 50%, by about 40%, by about 30%, by about 20%, by about 15%, by about 10%, by about 5%, or by about 1% for cell surface proteins in the animal (e.g., a mammal, such as a human) are fucosylated, as compared to an animal not receiving the carbacyclic compound as described herein, e.g., carbafucose. In some embodiments, protein fucosylation via α(1,4)-linkage is reduced by about 60%, by about 50%, by about 40%, by about 30%, by about 20%, by about 15%, by about 10%, by about 5%, or by about 1% for cell surface proteins in the animal (e.g., a mammal, such as a human) are fucosylated, as compared to an animal not receiving the carbacyclic compound as described herein, e.g., carbafucose.

In some embodiments, protein fucosylation via α(1,6)-linkage is reduced by about 60%, by about 50%, by about 40%, by about 30%, by about 20%, by about 15%, by about 10%, by about 5%, or by about 1% for cell surface proteins in the animal (e.g., a mammal, such as a human) are fucosylated, as compared to an animal not receiving the carbacyclic compound as described herein, e.g., carbafucose.

In some embodiments, fucosylation of white blood cells in the serum of the animal (e.g., a mammal, such as a human) is reduced by at least about 60%, at least about 50%, at least about 40%, at least about 30%, at least about 20%, at least about 15%, at least about 10%, or at least about 5%, as compared to an animal not receiving the carbacyclic compound as described herein, e.g., carbafucose. In some embodiments, fucosylation via α(1,3) linkages of white blood cells in the serum of the animal (e.g., a mammal, such as a human) is reduced by at least about 60%, at least about 50%, at least about 40%, at least about 30%, at least about 20%, at least about 15%, at least about 10%, or at least about 5%, as compared to an animal not receiving the carbacyclic compound as described herein, e.g., carbafucose. In some embodiments, fucosylation via α(1,4) linkages of white blood cells in the serum of the animal (e.g., a mammal, such as a human) is reduced by at least about 60%, at least about 50%, at least about 40%, at least about 30%, at least about 20%, at least about 15%, at least about 10%, or at least about 5%, as compared to an animal not receiving the carbacyclic compound as described herein, e.g., carbafucose.

In certain embodiments, only a minor amount of a fucose analog (or a metabolite or product of the carbacyclic compound as described herein, e.g., carbafucose) is incorporated into glycans (e.g., an N-glycan or the complex N-glycoside-linked sugar chain(s)) of an antibody or antibody derivative or other glycans of proteins. For example, in various embodiments, less than about 60%, less than about 40%, less than about 30%, less than about 20%, less than about 15%, less than about 10%, less than about 5%, or less than about 1% of the carbacyclic compound as described herein, e.g., carbafucose (or a metabolite or product of the carbacyclic compound as described herein) is incorporated into glycans of the antibodies in the serum of the animal, as compared to an animal not receiving the carbacyclic compound as described herein. In some embodiments, less than about 60%, less than about 40%, less than about 30%, less than about 20%, less than about 15%, less than about 10%, less than about 5%, or less than about 1% of the carbacyclic compound as described herein, e.g., carbafucose (or a metabolite or product of the carbacyclic compound as described herein) is incorporated into glycans of cell surface proteins of the animal, as compared to an animal not receiving the carbacyclic compound as described herein. In some embodiments, none of a carbacyclic compound as described herein, e.g., carbafucose (or a metabolite or product of the carbacyclic compound as described herein) is incorporated into glycans (e.g., an N-glycan or the complex N-glycoside-linked sugar chain(s)) of an antibody, antibody derivative or other glycans of proteins.

In some embodiments, less than about 60%, less than about 40%, less than about 30%, less than about 20%, less than about 15%, less than about 10%, less than about 5%, or less than about 1% of the carbacyclic compound as described herein e.g., carbafucose (or a metabolite or product of the carbacyclic compound as described herein) is incorporated into glycans of white blood cells in the serum of the animal, as compared to an animal not receiving the carbacyclic compound as described herein. In some embodiments, none of the carbacyclic compound as described herein e.g., carbafucose (or a metabolite or product of the carbacyclic compound as described herein) is incorporated into glycans of white blood cells in the serum of the animal, as compared to an animal not receiving the carbacyclic compound as described herein.

Carbacyclic Compounds and Uses Thereof

In some embodiments, a carbacyclic compound as described herein, e.g., carbafucose, can be useful in a variety of therapeutic and non-therapeutic applications. For example, the carbacyclic compounds and/or antibodies can be used as therapeutics. Antibody derivatives (e.g., a receptor-Fc fusion) can be used as a therapeutic molecule. In some embodiments, the antibody or antibody derivative is not conjugated to another molecule. In some embodiments, the antibody is conjugated to a suitable drug (e.g., an antibody drug conjugate) or other active agent. The antibodies and antibody derivatives can also be used for non-therapeutic purposes, such as diagnostic assays, prognostic assays, release assays and the like. Accordingly, the present disclosure provides, in part, a kit including a compound as described herein, such as a compound of Formula I, II, III, IV, V, VI, VII, VIII, IX, X, XI, XII, XIII, or XIV, e.g., carbafucose, together with instructions for use in diagnostic assays, prognostic assays, release assays, etc. or for inhibiting fucosylation in animals or cells.

In some embodiments, the carbacyclic compound can provide therapeutic benefit to patients suffering from a cancer, an autoimmune disease, an infectious disease, an inflammatory disease, or sickle cell disease. Accordingly, the present disclosure provides, in part, methods for treating a cancer, an autoimmune disease, an infectious disease, an inflammatory disease, or sickle cell disease by administering a compound as described herein, such as a compound of Formula I, II, Ill, IV, V, VI, VII, VIII, IX, X, XI, XII, XIII, or XIV, e.g., carbafucose, or a pharmaceutically acceptable salt thereof, to a mammal, such as a human. Accordingly, the present disclosure provides, in part, pharmaceutical compositions including a compound as described herein, such as a compound of Formula I, II, III, IV, V, VI, VII, VIII, IX, X, XI, XII, XIII, or XIV, e.g., carbafucose, or a pharmaceutically acceptable salt thereof, in combination with a pharmaceutically acceptable carrier, to a mammal, such as a human.

The subject may be suspected of having or at risk for having a cancer, an autoimmune disease, an infectious disease, an inflammatory disease, or sickle cell disease, be diagnosed with a cancer, an autoimmune disease, an infectious disease, an inflammatory disease, or sickle cell disease, or be a control subject that is confirmed to not have a cancer, an autoimmune disease, an infectious disease, an inflammatory disease, or sickle cell disease. Diagnostic methods for cancer, an autoimmune disease, an infectious disease, an inflammatory disease, or sickle cell disease and the clinical delineation of such diagnoses are known to those of ordinary skill in the art.

Cancers

A carbacyclic compound as described herein, can be useful for treating cancer in patients. Administration of a carbacyclic compound as described herein to an animal (e.g., a mammal, such as a human) in need thereof can result in inhibition of the multiplication of a tumor cell(s) or cancer cell(s), or treatment of cancer in an animal (e.g., a human patient). A carbacyclic compound as described herein can be used accordingly in a variety of settings for the treatment of animal cancers.

A carbacyclic compound as described herein can also be useful for enhancing the in vivo production of antibodies lacking fucosylation. Increasing the proportion of such antibodies against cancer targets in a patient can result in inhibition of the multiplication of a tumor cell(s) or cancer cell(s), or treatment of cancer in an animal (e.g., a human patient). A carbacyclic compound as described herein can be used accordingly in a variety of settings for the treatment of animal cancers.

Particular types of cancers that can be treated with a carbacyclic compound as described herein include, solid tumors and hematologic malignancies. Such cancers include, but are not limited to: (1) solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias, and (3) lymphomas such as Hodgkin's disease, non-Hodgkin's Lymphoma, multiple myeloma, Waldenstrom's macroglobulinemia, Heavy chain disease, and Polycythemia vera.

A cancer, including, but not limited to, a tumor, a metastasis, or any disease or disorder characterized by uncontrolled cell growth, can be treated or prevented by administration of a carbacyclic compound as described herein, to an animal (e.g., a mammal, such as a human) in need thereof. In some embodiments, the invention provides methods for treating or preventing cancer, comprising administering to an animal in need thereof an effective amount of a carbacyclic compound as described herein and optionally a chemotherapeutic agent. In one embodiment the chemotherapeutic agent is that with which treatment of the cancer has not been found to be refractory. In another embodiment, the chemotherapeutic agent is that with which the treatment of cancer has been found to be refractory. A carbacyclic compound as described herein can be administered to an animal that has also undergone surgery as treatment for the cancer.

In one embodiment, the additional method of treatment may be radiation therapy.

In a specific embodiment, a carbacyclic compound as described herein is administered concurrently with the chemotherapeutic agent or with radiation therapy. In another specific embodiment, the chemotherapeutic agent or radiation therapy is administered prior or subsequent to administration of a carbacyclic compound as described herein, preferably at least an hour, five hours, 12 hours, a day, a week, two weeks, three weeks, a month, or several months (e.g., up to three months), prior or subsequent to administration of a carbacyclic compound as described herein.

A chemotherapeutic agent can be administered over a series of sessions, and can be any one or a combination of the chemotherapeutic agents provided herein. With respect to radiation, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, the invention provides methods of treatment of cancer with a carbacyclic compound as described herein as an alternative to chemotherapy or radiation therapy, where the chemotherapy or the radiation therapy has proven or can prove too toxic, e.g., results in unacceptable or unbearable side effects, for the subject being treated. The animal being treated can, optionally, be treated with another cancer treatment such as surgery, radiation therapy or chemotherapy, depending on which treatment is found to be acceptable or bearable.

The present disclosure also provides methods for treating a cancer, comprising administering to an animal in need thereof an effective amount of a carbacyclic compound as described herein and a therapeutic agent that is an anti-cancer agent. Suitable anticancer agents include, but are not limited to, methotrexate, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbizine, topotecan, nitrogen mustards, Cytoxan, etoposide, 5-fluorouracil, BCNU, irinotecan, a camptothecin, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, the anti-cancer agent includes, but is not limited to: alkylating agents, nitrogen mustards (cyclophosphamide, Ifosfamide, trofosfamide, Chlorambucil), nitrosoureas (carmustine (BCNU), Lomustine (CCNU)), alkylsulphonates (busulfan, Treosulfan), triazenes (Dacarbazine), platinum containing compounds (cisplatin, oxaliplatin, carboplatin), plant alkaloids (vinca alkaloids—vincristine, Vinblastine, Vindesine, Vinorelbine), taxoids (paclitaxel, Docetaxol), DNA Topoisomerase Inhibitors, Epipodophyllins (etoposide, Teniposide, Topotecan, 9-aminocamptothecin, camptothecin), crisnatol, mitomycins (Mitomycin C); Anti-metabolites such as Anti-folates: DHFR inhibitors: methotrexate, Trimetrexate; IMP dehydrogenase Inhibitors: mycophenolic acid, Tiazofurin, Ribavirin, EICAR; Ribonuclotide reductase Inhibitors: hydroxyurea deferoxamine; Pyrimidine analogs: Uracil analogs: 5-Fluorouracil, Floxuridine, Doxifluridine, Ratitrexed; Cytosine analogs: cytarabine (ara C), Cytosine arabinoside, fludarabine; Purine analogs: mercaptopurine, Thioguanine; Hormonal therapies: Receptor antagonists: Anti-estrogen: Tamoxifen, Raloxifene, megestrol; LHRH agonists: goscrclin, Leuprolide acetate; Anti-androgens: flutamide, bicalutamide; Retinoids/Deltoids: Vitamin D3 analogs: EB 1089, CB 1093, KH 1060; Photodynamic therapies: vertoporfin (BPD-MA), Phthalocyanine, photosensitizer Pc4, Demethoxy-hypocrellin A (2BA-2-DMHA); Cytokines: Interferon-alpha, Interferon-gamma; Tumor necrosis factor: Others: Isoprenylation inhibitors: Lovastatin; Dopaminergic neurotoxins: 1-methyl-4-phenylpyridinium ion; Cell cycle inhibitors: staurosporine; Actinomycins: Actinomycin D, Dactinomycin; Bleomycins: bleomycin A2, Bleomycin B2, Peplomycin; Anthracyclines: daunorubicin, Doxorubicin (adriamycin), Idarubicin, Epirubicin, Pirarubicin, Zorubicin, Mitoxantrone; MDR inhibitors: verapamil; and Ca²⁺ ATPase inhibitors: thapsigargin.

In some embodiments, a carbacyclic compound as described herein can be used as an adjuvant, in combination with a cancer vaccine. The term “cancer vaccine” as used herein means a compound that selectively damages tumor cells by inducing and/or enhancing a specific immune response against the tumor cells. A cancer vaccine can be, for example, a medicament comprising a peptide, polypeptide or protein of a TAA or TSA, and pharmaceutical compositions containing a peptide, polypeptide or protein of a TAA or TSA. As used herein, TSA refers to a “tumor-specific antigen” and TAA refers to a tumor-associated antigen. TSAs are molecules unique to cancer cells. TAAs are molecules shared, but differently expressed, by cancer cells and normal cells.

The dosage of the cancer vaccine can be determined with appropriate modifications according to the extent of stimulation of an immune response against the vaccine. In general, it is between 0.01 and 100 mg/day/adult human, or preferably between 0.1 and 10 mg/day/adult human as an active principle. The cancer vaccine can be administered from once every few days to every few months. Administration can be carried out according to well-known methods for administrating a peptide, polypeptide or protein for medical use, such as subcutaneously, intravenously, or intramuscularly. In order to induce and/or enhance the immune response during administration, the peptide, polypeptide or protein can be used, in the presence or absence of an appropriate adjuvant, with or without linking to a carrier. The carrier is not limited as long as it exerts no harmful effect by itself onto the human body and is capable of enhancing antigenicity; cellulose, polymeric amino acids, albumin, and the like can be given as examples of carriers. Adjuvants can be those used in general for peptide vaccine inoculation, and a Freund incomplete adjuvant (FIA), aluminum adjuvant (ALUM), Bordetella pertussis vaccine, mineral oil, and the like can be given as examples. In addition, the formulation can be suitably selected by applying a suitable well-known method for formulating a peptide, polypeptide or protein.

Otherwise, an effective cancer vaccine effect can be obtained also by collecting a fraction of mononuclear cells from the peripheral blood of a patient, incubating them with the peptide, polypeptide or protein of the present invention, and then returning the fraction of mononuclear cells in which induction of CTL and/or activation of CTL was observed, into the blood of the patient. A carbacyclic compound as described herein can be co-administered during or after re-administration of the mononuclear cells. Culture conditions, such as mononuclear cell concentration, concentration of the peptide, polypeptide or proteins, culture time, and the like, can be determined by simply repeating studies. A substance having a capability to enhance the growth of lymphocytes, such as interleukin-2, may be added during culturing.

Autoimmune Diseases

A carbacyclic compound as described herein, e.g., carbafucose, can be useful for modulating an autoimmune disease or for treating an autoimmune disease, so as to decrease symptoms and/or the autoimmune response. A carbacyclic compound as described herein can be used accordingly in a variety of settings for the treatment of an autoimmune disease in an animal.

In one embodiment, a carbacyclic compound as described herein can down-regulate or down-modulate an auto-immune antibody associated with a particular autoimmune disease.

Particular types of autoimmune diseases that can be treated with a carbacyclic compound as described herein include, but are not limited to, Th2-lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); activated B lymphocyte-related disorders (e.g., systemic lupus erythematosus, Goodpasture's syndrome, rheumatoid arthritis, and type I diabetes); Active Chronic Hepatitis, Addison's Disease, Allergic Alveolitis, Allergic Reaction, Allergic Rhinitis, Alport's Syndrome, Anaphlaxis, Ankylosing Spondylitis, Anti-phosholipid Syndrome, Arthritis, Ascariasis, Aspergillosis, Atopic Allergy, Atropic Dermatitis, Atropic Rhinitis, Behcet's Disease, Bird-Fancier's Lung, Bronchial Asthma, Caplan's Syndrome, Cardiomyopathy, Celiac Disease, Chagas' Disease, Chronic Glomerulonephritis, Cogan's Syndrome, Cold Agglutinin Disease, Congenital Rubella Infection, CREST Syndrome, Crohn's Disease, Cryoglobulinemia, Cushing's Syndrome, Dermatomyositis, Discoid Lupus, Dressler's Syndrome, Eaton-Lambert Syndrome, Echo virus Infection, Encephalomyelitis, Endocrine opthalmopathy, Epstein-Barr Virus Infection, Equine Heaves, Erythematosis, Evan's Syndrome, Felty's Syndrome, Fibromyalgia, Fuch's Cyclitis, Gastric Atrophy, Gastrointestinal Allergy, Giant Cell Arteritis, Glomerulonephritis, Goodpasture's Syndrome, Graft v. Host Disease, Graves' Disease, Guillain-Barre Disease, Hashimoto's Thyroiditis, Hemolytic Anemia, Henoch-Schonlein, Purpura Idiopathic Adrenal Atrophy, Idiopathic Pulmonary Fibritis, IgA Nephropathy, Inflammatory Bowel Diseases, Insulin-dependent Diabetes Mellitus, Juvenile Arthritis, Juvenile Diabetes Mellitus (Type 1), Lambert-Eaton Syndrome, Laminitis, Lichen Planus, Lupoid Hepatitis, Lupus Lymphopenia, Meniere's Disease, Mixed Connective Tissue Disease, Multiple Sclerosis, Myasthenia Gravis, Pernicious Anemia, Polyglandular Syndromes, Presenile Dementia, Primary Agammaglobulinemia, Primary Biliary Cirrhosis, Psoriasis, Psoriatic Arthritis, Raynauds Phenomenon, Recurrent Abortion, Reiter's Syndrome, Rheumatic Fever, Rheumatoid Arthritis, Sampter's Syndrome, Schistosomiasis, Schmidt's Syndrome, Scleroderma, Shulman's Syndrome, Sjogren's Syndrome, Stiff-Man Syndrome, Sympathetic Ophthalmia, Systemic Lupus Erythematosis, Takayasu's Arteritis, Temporal Arteritis, Thyroiditis, Thrombocytopenia, Thyrotoxicosis, Toxic Epidermal Necrolysis Type B, Insulin Resistance Type I Diabetes Mellitus, Ulcerative Colitis, Uveitis Vitiligo, Waldenstrom's Macroglobulemia, and Wegener's Granulomatosis.

The present disclosure also provides methods for treating an autoimmune disease, comprising administering to an animal (e.g., a mammal) in need thereof an effective amount of a carbacyclic compound as described herein and optionally a therapeutic agent that known for the treatment of an autoimmune disease. In one embodiment, the anti-autoimmune disease agent includes, but is not limited to cyclosporine, cyclosporine A, mycophenylate, mofetil, sirolimus, tacrolimus, enanercept, prednisone, azathioprine, methotrexate, cyclophosphamide, prednisone, aminocaproic acid, chloroquine, hydroxychloroquine, hydrocortisone, dexamethasone, chlorambucil, DHEA, danazol, bromocriptine, meloxicam or infliximab.

Infectious Diseases

A carbacyclic compound as described herein, e.g., carbafucose, can be useful for enhancing an immune response that results in increased killing or inhibition of the multiplication of a cell that produces an infectious disease or for treating an infectious disease. A carbacyclic compound as described herein can be used accordingly in a variety of settings for the treatment of an infectious disease in an animal.

In one embodiment, a carbacyclic compound as described herein can enhance an immune response, resulting in kill or inhibit, or increased killing or inhibition, of the multiplication of cells that produce a particular infectious disease.

Particular types of infectious diseases that can be treated with a carbacyclic compound as described herein include, but are not limited to, (1) Bacterial Diseases: Diptheria, Pertussis, Occult Bacteremia, Urinary Tract Infection, Gastroenteritis, Cellulitis, Epiglottitis, Tracheitis, Adenoid Hypertrophy, Retropharyngeal Abcess, Impetigo, Ecthyma, Pneumonia, Endocarditis, Septic Arthritis, Pneumococcal, Peritonitis, Bactermia Meningitis, Acute Purulent Meningitis, Urethritis, Cervicitis, Proctitis, Pharyngitis, Salpingitis, Epididymitis, Gonorrhea, Syphilis, Listeriosis, Anthrax, Nocardiosis, Salmonella, Typhoid Fever, Dysentery, Conjuntivitis, Sinusitis, Brucellosis, Tullaremia, Cholera, Bubonic Plague, Tetanus, Necrotizing Enteritis, Actinomycosis Mixed Anaerobic Infections, Syphilis, Relapsing Fever, Leptospirosis, Lyme Disease, Rat Bite Fever, Tuberculosis, Lymphadenitis, Leprosy, Chlamydia, Chlamydial Pneumonia, Trachoma, Inclusion Conjunctivitis, Systemic; (2) Fungal Diseases: Histoplamosis, Coccicidiodomycosis, Blastomycosis, Sporotrichosis, Cryptococcsis, Systemic Candidiasis, Aspergillosis, Mucormycosis, Mycetoma, Chromomycosis; (3) Rickettsial Diseases: Typhus, Rocky Mountain Spotted Fever, Ehrlichiosis, Eastern Tick-Borne Rickettsioses, Rickettsialpox, Q Fever, and Bartonellosis; (4) Parasitic Diseases: Malaria, Babesiosis, African Sleeping Sickness, Chagas' Disease, Leishmaniasis, Dum-Dum Fever, Toxoplasmosis, Meningoencephalitis, Keratitis, Entamebiasis, Giardiasis, Cryptosporidiasis, Isosporiasis, Cyclosporiasis, Microsporidiosis, Ascariasis, Whipworm Infection, Hookworm Infection, Threadworm Infection, Ocular Larva Migrans, Trichinosis, Guinea Worm Disease, Lymphatic Filariasis, Loiasis, River Blindness, Canine Heartworm Infection, Schistosomiasis, Swimmer's Itch, Oriental Lung Fluke, Oriental Liver Fluke, Fascioliasis, Fasciolopsiasis, Opisthorchiasis, Tapeworm Infections, Hydatid Disease, Alveolar Hydatid Disease; (5) Viral Diseases: Measles, Subacute sclerosing panencephalitis, Common Cold, Mumps, Rubella, Roseola, Fifth Disease, Chickenpox, Respiratory syncytial virus infection, Croup, Bronchiolitis, Infectious Mononucleosis, Poliomyelitis, Herpangina, Hand-Foot-and-Mouth Disease, Bornholm Disease, Genital Herpes, Genital Warts, Aseptic Meningitis, Myocarditis Pericarditis, Gastroenteritis, Acquired Immunodeficiency Syndrome (AIDS), Reye's Syndrome, Kawasaki Syndrome, Influenza, Bronchitis, Viral “Walking” Pneumonia, Acute Febrile Respiratory Disease, Acute pharyngoconjunctival fever, Epidemic keratoconjunctivitis, Herpes Simplex Virus 1 (HSV-1), Herpes Simples Virus 2 (HSV-2), Shingles, Cytomegalic Inclusion Disease, Rabies, Progressive Multifocal Leukoencephalopathy, Kuru, Fatal Familial Insomnia, Creutzfeldt-Jakob Disease, Gerstmann-Straussler-Scheinker Disease, Tropical Spastic Paraparesis, Western Equine Encephalitis, California Encephalitis, St. Louis Encephalitis, Yellow Fever, Dengue Lymphocytic choriomeningitis, Lassa Fever, Hemorrhagic Fever, Hantvirus, Pulmonary Syndrome, Marburg Virus Infections, Ebola Virus Infections, smallpox and COVID-19.

The present disclosure also provides methods for treating an infectious disease, by administering to an animal (e.g., a mammal) in need thereof a carbacyclic compound as described herein and optionally a therapeutic agent that is an anti-infectious disease agent. In one embodiment, the anti-infectious disease agent is, but not limited to: (1) Antibacterial Agents: p-Lactam Antibiotics: Penicillin G, Penicillin V, Cloxacilliin, Dicloxacillin, Methicillin, Nafcillin, Oxacillin, Ampicillin, Amoxicillin, Bacampicillin, Azlocillin, Carbenicillin, Mezlocillin, Piperacillin, Ticarcillin; Aminoglycosides: Amikacin, Gentamicin, Kanamycin, Neomycin, Netilmicin, Streptomycin, Tobramycin; Macrolides: Azithromycin, Clarithromycin, Erythromycin, Lincomycin, Clindamycin; Tetracyclines: Demeclocycline, Doxycycline, Minocycline, Oxytetracycline, Tetracycline; Quinolones: Cinoxacin, Nalidixic Acid, Fluoroquinolones: Ciprofloxacin, Enoxacin, Grepafloxacin, Levofloxacin, Lomefloxacin, Norfloxacin, Ofloxacin, Sparfloxacin, Trovafloxicin; Polypeptides: Bacitracin, Colistin, Polymyxin B; Sulfonamides: Sulfisoxazole, Sulfamethoxazole, Sulfadiazine, Sulfamethizole, Sulfacetamide; Miscellaneous Antibacterial Agents: Trimethoprim, Sulfamethazole, Chloramphenicol, Vancomycin, Metronidazole, Quinupristin, Dalfopristin, Rifampin, Spectinomycin, Nitrofurantoin; Antiviral Agents: General Antiviral Agents: Idoxuradine, Vidarabine, Trifluridine, Acyclovir, Famcicyclovir, Pencicyclovir, Valacyclovir, Gancicyclovir, Foscarnet, Ribavirin, Amantadine, Rimantadine, Cidofovir; Antisense Oligonucleotides; mmunoglobulins; Inteferons; Drugs for HIV infection: Zidovudine, Didanosine, Zalcitabine, Stavudine, Lamivudine, Nevirapine, Delavirdine, Saquinavir, Ritonavir, Indinavir and Nelfinavir.

Inflammatory Diseases

Inflammation involves the reaction of vascularized living tissue to injury. By way of example, although fucose containing epitopes that mediate cell adhesion are important to the body's anti-infective immune response, in other circumstances, fucose containing epitopes that mediate cell adhesion may be undesirable or excessive, resulting in tissue damage instead of repair. For example, many pathologies (such as autoimmune and inflammatory diseases, shock and reperfusion injuries) involve abnormal adhesion of white blood cells that are mediated by fucose-containing epitopes. Therefore, inflammation affects blood vessels and adjacent tissues in response to an injury or abnormal stimulation by a physical, chemical, or biological agent. Examples of inflammatory diseases or disorders include, without limitation, vascular inflammatory disease, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the carbacyclic compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischaemic episodes, prevention of restinosis associated with vascular stenting, and for undesirable angiogenesis, for example, angiogenesis associated with tumor growth.

Other Therapeutic Agents

The methods provided in accordance with the present disclosure can further include the administration of a carbacyclic compound as described herein, e.g., carbafucose, and a therapeutic agent or pharmaceutically acceptable salts or solvates thereof. The carbacyclic compound as described herein and the therapeutic agent can act additively or, more preferably, synergistically. In a preferred embodiment, a composition comprising a carbacyclic compound as described herein is administered concurrently with the administration of one or more therapeutic agent(s), which can be part of the same composition or in a different composition from that comprising the carbacyclic compound as described herein. In another embodiment, a carbacyclic compound as described herein is administered prior to or subsequent to administration of the therapeutic agent(s).

In the present methods for treating cancer, an autoimmune disease or an infectious disease, the therapeutic agent also can be an antiemetic agent. Suitable antiemetic agents include, but are not limited to, metoclopromide, domperidone, proclorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinols, thiethylperazine, thioproperazine and tropisetron.

In another embodiment, the therapeutic agent can be an hematopoietic colony stimulating factor. Suitable hematopoietic colony stimulating factors include, but are not limited to, filgrastim, sargramostim, molgramostim and erythropoietin alfa.

In still another embodiment, the therapeutic agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefenamic acid, nabumetone, naproxen, piroxicam and sulindac.

Compositions, Dosages, and Administration

A carbacyclic compound as described herein, e.g., carbafucose, can be formulated for therapeutic applications. The carbacyclic compounds can be formulated as pharmaceutical compositions including a therapeutically or prophylactically effective amount of the antibody or derivative and one or more pharmaceutically compatible (acceptable) ingredients.

An “effective amount” of carbacyclic compound as described herein, e.g., carbafucose, includes a therapeutically effective amount or a prophylactically effective amount or a nutritionally effective amount. A “therapeutically effective amount” refers to an amount effective, at dosages and for periods of time necessary, to achieve the desired therapeutic result. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. An exemplary range for therapeutically or prophylactically effective amounts of a compound may be about 5- about 50 mg/day/kg of body weight of the subject e.g., a human.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, the carbacyclic compound should be used without causing substantial toxicity. Toxicity of the carbacyclic compound can be determined using standard techniques, for example, by testing in cell cultures or experimental animals or subjects and determining the therapeutic index, i.e., the ratio between the LD50 (the dose lethal to 50% of the population) and the ED50 (the minimum effective dose for 50% of the population) for non-human animals or the ratio between the TD50 (the dose toxic to 50% of the population) and the ED50 (the minimum effective dose for 50% of the population) for humans. The maximum tolerated dose (MTD) is the highest regularly administered dose of a compound or composition that does not cause overt toxicity (e.g. does not cause unacceptable side effects) in a subject study over a period of time. The subject may be a human, or an animal, such as a mouse or a rat, for example.

The regularly administered dose may be a daily dose, administered as a single bolus; alternately the daily dose may be divided into two or more partial doses so that the subject receives the total daily dose over time. The period of time of the study may vary from a few days to a few months, for example about 10, 20, 30, 60, 90 or 120 days, or any value therebetween. Examples of overt toxicity may include, but are not limited to, appreciable death of cells or organ dysfunction, toxic manifestations that are predicted materially to reduce the life span of the subject, or 10% or greater retardation of body weight gain. In some embodiments, the carbacyclic compound may be provided together with other compounds (for example, nucleic acid molecules, small molecules, peptides, or peptide analogues), in the presence of a liposome, an adjuvant, or any pharmaceutically or physiologically acceptable carrier, in a form suitable for administration to humans or animals. If desired, treatment with the carbacyclic compound according to the invention may be combined with more traditional and existing therapies for the condition to be treated.

Conventional pharmaceutical or non-pharmaceutical formulation practice may be employed to provide suitable formulations or compositions to administer the carbacyclic compound to patients suffering from the condition to be treated. For example, a pharmaceutical or non-pharmaceutical composition typically includes one or more carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable excipients include, for example, amino acids, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin. Such compositions will typically contain a therapeutically effective amount of the protein, typically in purified form, together with a suitable amount of carrier so as to provide the form for proper administration to the patient. The formulations correspond to the mode of administration.

Any appropriate route of administration may be employed, for example, parenteral, intravenous, subcutaneous, intramuscular, intracranial, intraorbital, ophthalmic, intraventricular, intracapsular, intraspinal, intracistemal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, Remington's Pharmaceutical Sciences. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. Typically, compositions for intravenous administration are solutions in sterile isotonic aqueous buffer. When necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. When the pharmaceutical is to be administered by infusion, it can be dispensed with an infusion bottle containing sterile pharmaceutical grade water or saline. When the pharmaceutical is administered by injection, an ampoule of sterile water for injection or saline can be provided so that the ingredients can be mixed prior to administration.

The present invention will be further illustrated in the following examples.

### EXAMPLES

### General Experimental Details

All anhydrous reactions described were performed under an atmosphere of nitrogen using flamedried glassware. Normal phase column chromatography was carried out with 230-400 mesh silica gel (Silicycle, SiliaFlash® P60). Concentration and removal of trace solvents was done with a Bũchi rotary evaporator using a dry ice/acetone condenser and vacuum applied from a Bũchi V-500 pump.

All reagents and starting materials were purchased from Sigma Aldrich, Alfa Aesar, TCl America, Arcos or Carbosynth and were used without further purification. All solvents were purchased from Sigma Aldrich, EMD, Anachemia, Caledon, Fisher or ACP and used without further purification unless otherwise specified. CH₂Cl₂ was freshly distilled over CaH₂; Tetrahydrofuran (THF) was freshly distilled over Na metal/benzophenone. Cold temperatures were maintained by use of the following conditions: 0° C., ice-water bath; −78° C., acetone-dry ice bath; temperatures between −78° C. and 0° C. required for longer reaction times were maintained with a Neslab Cryocool Immersion Cooler (CC-100 II) in a 2-propanol bath.

Nuclear magnetic resonance (NMR) spectra were recorded using CDCl₃ or CD₃OD. Signal positions (6) are given in parts per million from tetramethylsilane (δ0) and were measured relative to the signal of the solvent (¹H NMR: CDCl₃: δ 7.26, CD₃OD: δ 3.31, D₂O: δ 4.79; ¹³C NMR: CDCl₃: δ 77.16, CD₃OD: δ 49.00). Coupling constants (J values) are given in Hertz (Hz) and are reported to the nearest 0.1 Hz. ¹H NMR spectral data are tabulated in the order: multiplicity (s, singlet; d, doublet; t, triplet; q, quartet; m, multiplet; br., broad), coupling constants, number of protons.

Infrared (IR) spectra were recorded on a Perkin Elmer Spectrum Two™ Fourier transform spectrometer with neat samples. Only selected, characteristic absorption data are provided for each compound.

High resolution mass spectra were performed on an Agilent 6210 TOF LC/MS using ESI-MS technique.

Optical rotation was measured on a Perkin Elmer 341 Polarimeter at 589 nm.

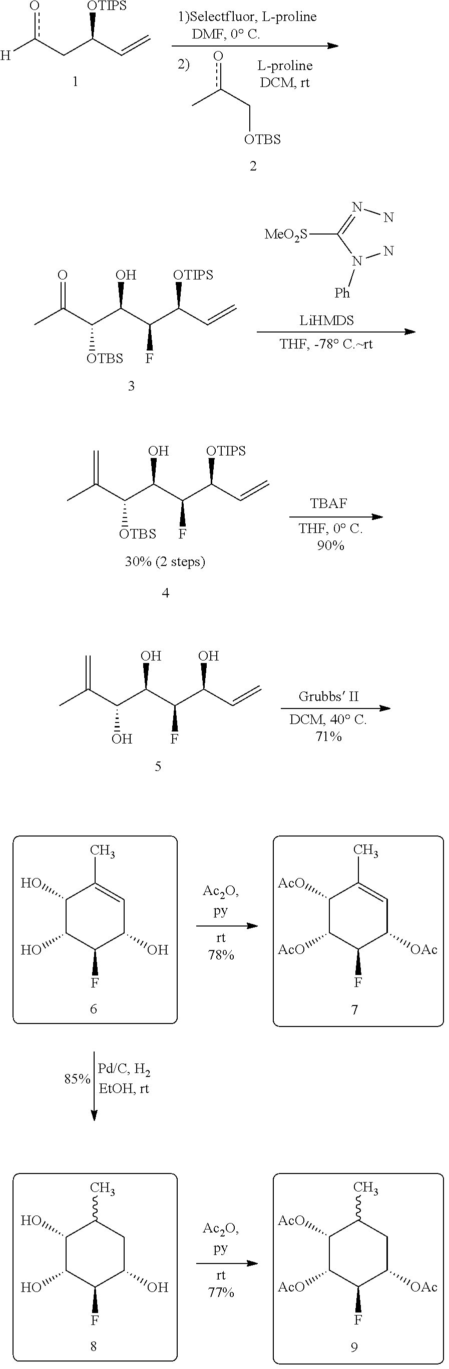

Synthesis of diene 4. To a solution of 1 (2.0 mmol, 1 equiv) in DMF (20 mL) at 5° C. were added Selectfluor® (2.0 mmol, 1 equiv) and (L)-proline (2.0 mmol, 1 equiv) The mixture was stirred at 5° C. for 1 h, treated with H₂O, then extracted with Et₂O. The combined organic layers were washed with brine and then dried over Na₂SO₄. The solvents were removed in vacuo and the residue was redissolved in CH₂Cl₂ (10 mL). (L)-proline (1.6 mmol, 0.8 equiv) and 1-((tert-butyldimethylsilyl)oxy)propan-2-one 2 (2.6 mmol, 1.3 equiv) were then added at 0° C. The mixture was warmed to room temperature and stirred for 48 h. The resulting mixture was then treated with H₂O and extracted with Et₂O. The combined organic layers were washed with brine and then dried over Na₂SO₄. The solvents were removed in vacuo and the residue was dissolved in THF (6 mL). In another flask, LiHMDS (1.0 M in THF, 5.0 mmol, 2.5 equiv) was added dropwise to a cooled (−78° C.) solution of 5-(methanesulfonyl)-1-phenyl-1 H-tetrazole (5.0 mmol, 2.5 equiv) in THF (14 mL) and stirred at −78° C. for 30 min. Then the above solution of ketone 3 in THF (6 mL) was added dropwise at −78° C. and the mixture was stirred for another 1 h before quenching with H₂O. The mixture was extracted with Et₂O and the combined organic layers were washed with brine and then dried over Na₂SO₄. The solvents were removed in vacuo and the residue was purified by flash column chromatography (pentane:ethyl ether, 25:1) to give compound 4 as a yellow oil (276 mg, 30% for 2 steps). [α]_{D}=−14.5 (c=1.1, CHCl₃); ¹H NMR (400 MHz, CDCl₃) δ 5.94 (ddd, J=6.5, 10.5, 17.1 Hz, 1H), 5.40 (dt, J=1.4, 17.3 Hz, 1H), 5.23 (m, 1H), 5.01 (m, 1H), 4.68-4.46 (m, 2H), 4.13 (d, J=8.8 Hz, 1H), 3.56 (ddd, J=5.4, 8.9, 27.8 Hz, 1H), 1.79 (d, J=5.4 Hz, 1H), 1.71 (s, 3H), 1.07 (s, 12H), 1.06 (s, 6H), 0.88 (s, 9H), 0.08 (s, 3H), 0.02 (s, 3H); ¹³C NMR (101 MHz, CDCl₃) δ 145.2, 137.2 (d, J=7.0 Hz), 117.4, 115.3, 93.3 (d, J=183.0 Hz), 76.4 (d, J=4.2 Hz), 74.5 (d, J=22.6 Hz), 70.5 (d, J=18.2 Hz), 25.9, 18.2, 18.1, 18.1, 18.1, 16.4, 12.6, −4.6, −5.3, −5.3; ¹⁹F NMR (377 MHz, CDCl₃) δ −208.7 (ddd, J=12.6, 27.8, 46.3 Hz); IR (neat) v 3570, 2939, 1641, 1466, 1087, 829 cm⁻¹; HRMS (ESI-TOF) m/z: [M+H]⁺ Calcd for C₂₄H₅₀FO₃Si₂ 461.3277; Found 461.3274.

Synthesis of triol 5. To a solution of 4 (0.5 mmol, 1 equiv) in THF (5 mL) at 0° C. were added TBAF (1.5 mmol, 3 equiv). The mixture was stirred at 0° C. for 2 h. The solvents were removed in vacuo and the residue was purified by flash column chromatography (pentane:ethyl acetate, 1:1) to give compound 5 as a colorless oil (85 mg, 90%).

Synthesis of fluorotriol 6. To a solution of 5 (0.1 mmol, 1 equiv) in CH₂Cl₂ (5 mL) at rt were added Grubbs' II catalyst (0.01 mmol, 0.1 equiv). The mixture was heated to 40° C. under argon and maintained at 40° C. for 2 h. The reaction was cooled to room temperature and concentrated in vacuo. The residue was then purified by flash column chromatography (pentane:ethyl acetate, 6:1 then 1:1) to give compound 6 as a colorless oil (12.1 mg, 71%). [α]_{D}=−79.5 (c=0.5, CH₃OH); ¹H NMR (400 MHz, CD₃OD) δ 5.36 (m, 1H), 4.44 (ddd, J=7.2, 10.4, 54.3 Hz, 1H), 4.16 (m, 1H), 3.99 (t, J=4.7 Hz, 1H), 3.63 (ddd, J=4.4, 8.5, 10.5 Hz, 1 H), 1.83 (t, J=1.8 Hz, 3H); ¹³C NMR (151 MHz, CD₃OD) δ136.9 (d, J=2.4 Hz), 126.9 (d, J=9.7 Hz), 96.0 (d, J=177.7 Hz), 72.4 (d, J=7.7 Hz), 71.7 (d, J=20.6 Hz), 71.1 (d, J=16.0 Hz), 20.6; ¹⁹F NMR (377 MHz, CD₃OD) δ −205.2; IR (cast film) v 3728, 3691, 2919, 1718, 1443, 967 cm⁻¹; HRMS (ESI-TOF) m/z: [M−H]⁻ Calcd for C₇H₁₀FO₃ 161.0619; Found 161.0617.

Synthesis of acetate 7. 6 (0.01 mmol) was dissolved in Ac₂O (25 μL) and pyridine (25 μL) and stirred at rt for 12 h. The solvents were removed in vacuo and the residue was purified by flash column chromatography (pentane:ethyl acetate, 2:1) to give compound 7 as a colorless oil (2.3 mg, 78%).

Synthesis of fluorotriol 8. A mixture of 6 (0.04 mmol, 1 equiv) and 10% Pd/C (0.004 mmol, 0.1 equiv) in EtOH (0.4 mL) was stirred at rt under H₂ (2 atm). After 12 h, the reaction mixture was filtered through Celite and the filtrate was evaporated in vacuo. The residue was purified by flash column chromatography (pentane:ethyl acetate, 2:1) to give compound 8 as a colorless oil (5.6 mg, 85%, ˜1.5:1 mixture of diastereomers). ¹⁹F NMR (377 MHz, CD₃OD): δ −203.7, −203.8.

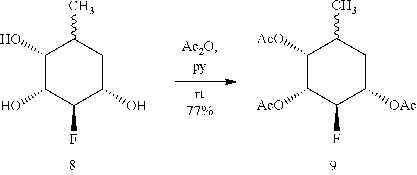

Synthesis of acetate 9. 8 (0.01 mmol) was dissolved in Ac₂O (25 μL) and pyridine (25 μL) and stirred at rt for 12 h. The solvents were removed in vacuo and the residue was purified by flash column chromatography (pentane:ethyl acetate, 2:1) to give compound 9 as a colorless oil (2.2 mg, 77%).

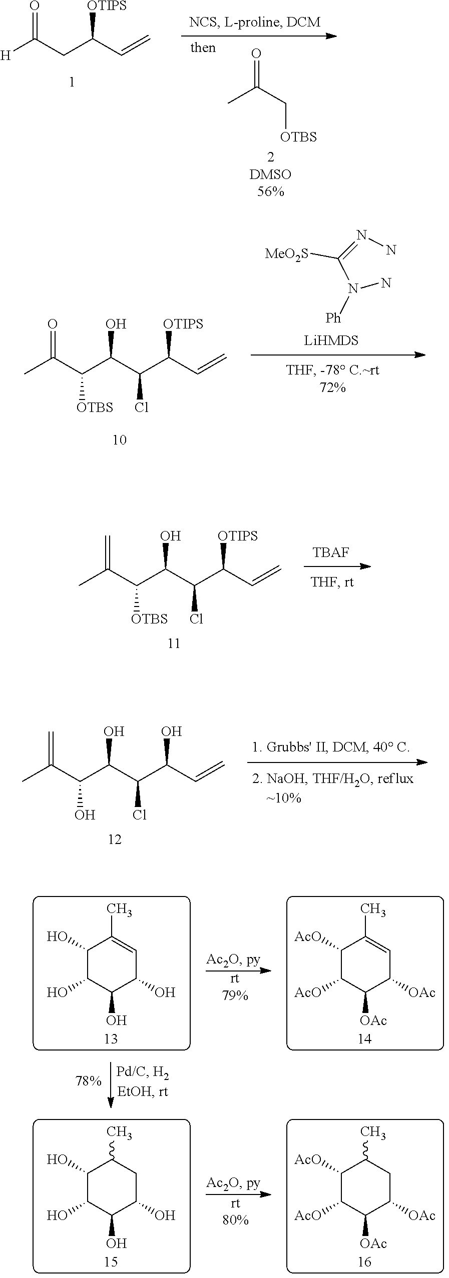

Synthesis of ketone 10. To a solution of aldehyde 1 (2.0 mmol, 1 equiv) in CH₂Cl₂ (5 mL) at 0° C. was added NCS (2.2 mmol, 1.1 equiv) and L-Proline (1.6 mmol, 0.8 equiv). The reaction mixture was left at 0° C. for 30 min, and ketone 2 (4 mmol, 2 equiv) in 5.0 mL of DMSO was added at 0° C. The reaction mixture was warmed to room temperature and stirred for 12 h. The reaction mixture was diluted with CH₂Cl₂, washed with water, dried with Na₂SO₄, filtered, and concentrated under reduced pressure. The residue was purified by flash column chromatography (pentane:ethyl ether, 8:1) to give compound 10 as a yellow oil (536 mg, 56%). [α]_{D}=−25.3 (c=1.19, CHCl₃); ¹H NMR (600 MHz, CDCl₃) δ 5.66-5.58 (m, 4H), 5.36-5.27 (m, 2H), 4.83 (dd, J=10.5, 3.0 Hz, 1H), 4.41 (m, 1H), 2.14 (s, 3H), 2.08 (s, 3H), 2.06 (m, 1H), 1.97 (s, 3H), 1.92-1.78 (m, 2H), 1.24 (s, 18H), 0.95 (d, J=6.6 Hz, 3 H); ¹³C NMR (151 MHz, CDCl₃) δ 209.4, 137.7, 117.8, 78.5, 76.9, 71.4, 64.8, 25.7, 25.4, 18.2, 18.1, 17.8, 12.6, −4.8, −5.0; IR (neat) v 3522, 2994, 1721, 1099, 838 cm⁻¹; HRMS (ESI-TOF) m/z: [M+NH₄]⁺ Calcd for C₂₃H₅₁NClO₄Si₂ 496.3040; Found 496.3045.

Synthesis of diene 11. LiHMDS (1.0 M in THF, 2.7 mmol, 2 equiv) was added dropwise to a cooled (−78° C.) solution of 5-(methanesulfonyl)-1-phenyl-1 H-tetrazole (2.7 mmol, 2 equiv) in THF (9.5 mL) and stirred at −78° C. for 30 min. Then 10 (1.35 mmol, 1 equiv) in THF (4 mL) was added dropwise at −78° C. and the mixture was stirred for another 1 h before quenching with H₂O. The mixture was extracted with Et₂O and the combined organic layers were washed with brine and then dried over Na₂SO₄. The solvents were removed in vacuo and the residue was purified by flash column chromatography (pentane:ethyl ether, 20:1) to give compound 11 as a yellow oil (464 mg, 72%). [α]_{D}=−15.1 (c=0.96, CHCl₃); ¹H NMR (500 MHz, CDCl₃) δ 6.09 (ddd, J=7.2, 10.3, 17.4 Hz, 1H), 5.31 (dt, J=1.3, 17.3 Hz, 1H), 5.22 (dt, J=1.0, 10.3 Hz, 1H), 5.0 (m, 1H), 4.54 (m, 1H), 4.33 (d, J=5.4 Hz, 1H), 4.04 (d, J=8.6 Hz, 1H), 3.98 (dd, J=5.6, 8.7 Hz, 1H), 2.12 (d, J=5.5 Hz, 1 H), 1.73 (s, 3H), 1.07 (s, 12H), 1.06 (s, 6H), 0.88 (s, 9H), 0.10 (s, 3H), 0.03 (s, 3 H); ¹³C NMR (126 MHz, CDCl₃) δ 145.1, 138.3, 117.2, 115.4, 77.5, 77.2, 70.4, 65.3, 25.9, 18.2, 18.2, 16.6, 12.6, −4.6, −5.0; IR (neat) v 3661, 2957, 1572, 1462, 1084, 836 cm⁻¹; HRMS (ESI-TOF) m/z: [M+H]⁺ Calcd for C₂₄H₅₀ClO₃Si₂ 477.2982; Found 477.2977.

Synthesis of triol 12. To a solution of 11 (1.0 mmol, 1 equiv) in THF (10 mL) at 0° C. were added TBAF (3.0 mmol, 3.0 equiv). The mixture was stirred at 0° C. for 2 h. The solvents were removed in vacuo and the residue was purified by flash column chromatography (pentane:ethyl acetate, 1:1) to give compound 12 as a yellow oil (169 mg, 82%).

Synthesis of tetraol 13. To a solution of 12 (0.5 mmol, 1 equiv) in CH₂Cl₂ (25 mL) at rt was added Grubbs' II catalyst (0.05 mmol, 0.1 equiv). The mixture was heated to 40° C. under argon and maintained at 40° C. for 1 h. The reaction was cooled to room temperature and the reaction mixture was filtered through Celite. The filtrate was evaporated in vacuo. The mixture was redissolved in THF (2.5 mL)/NaOH (2.0 M, 0.5 mL). The reaction was heated to reflux for 12 h. The reaction was cooled to room temperature. The solvents were removed in vacuo and the residue was purified by flash column chromatography (CH₂Cl₂:MeOH, 20:1 then 4:1) to give compound 13 as a colorless oil (8.0 mg, 10%). [α]_{D}=−61.5 (c=0.9, CH₃OH); ¹H NMR (400 MHz, CD₃OD) δ 5.40 (m, 1H), 3.95 (d, J=4.3 Hz, 1H), 3.88 (m, 1H), 3.57 (dd, J=7.6, 10.5 Hz, 1H), 3.38 (dd, J=4.3, 10.5 Hz, 1H), 1.82 (t, J=1.7 Hz, 1H); ¹³C NMR (151 MHz, CD₃OD) δ 136.3, 128.3, 74.1, 73.6, 72.8, 72.0, 20.8; IR (cast film) v3724, 3698, 2349, 1664, 1436, 1084 cm⁻¹; HRMS (ESI-TOF) m/z: [M+NH₄]⁺ Calcd for C₇H₁₆NO₄ 178.1074; Found 178.1075.

Synthesis of acetate 14. 13 (0.01 mmol) was dissolved in Ac₂O (25 μL) and pyridine (25 μL) and stirred at rt for 12 h. The solvents were removed in vacuo and the residue was purified by flash column chromatography (pentane:ethyl acetate, 3:1) to give compound 14 as a colorless oil (2.6 mg, 79%).

Synthesis of 15. A mixture of 13 (0.025 mmol, 1 equiv) and 10% Pd/C (0.0025 mmol, 0.1 equiv) in EtOH (0.25 mL) was stirred at rt under H₂ (2 atm). After 12 h, the reaction mixture was filtered through Celite and the filtrate was evaporated in vacuo. The residue was purified by flash column chromatography (CH₂Cl₂:MeOH, 20:1 then 5:1) to give 15 as a colorless oil (3.0 mg, 78%, 1.5:1 mix of diastereomers in favor of 1-21). The diastereomers could be separated by column chromatography (20% MeOH in EtOAc) to afford 1-21. [α]_{D}²⁰=+22.3 (c=0.05, CH₃OH); ¹H NMR (600 MHz, CD₃OD) δ3.68 (brs, 1H), 3.48-3.45 (m, 1H), 3.39-3.35 (m, 1H), 3.27 (dd, J=9.6, 3.0 Hz, 1H), 1.65-1.60 (m, 1H), 1.57-1.48 (m, 2H), 1.02 (d, J=6.8 Hz, 3H); ¹³C NMR (151 MHz, CD₃OD): δ 76.5, 76.4, 74.9, 73.8, 35.8, 33.1, 17.8; IR (neat) v3368, 2958, 2928, 2857, 1731, 1668, 1462, 1261, 1067, 1022, 799 cm⁻¹; HRMS (ESI-TOF) m/z: [M+Na]⁺ Calcd for C₇H₁₄NaO₄ 185.0784; Found 185.0784.

Synthesis of acetate 16. 15 (0.01 mmol) was dissolved in Ac₂O (25 μL) and pyridine (25 μL) and stirred at rt for 12 h. The solvents were removed in vacuo and the residue was purified by flash column chromatography (pentane:ethyl acetate, 3:1) to give compound 16 as a colorless oil (2.6 mg, 80%).

Glycan Analysis

Expi-CHO cells were treated with compounds 16, 7, 9, 13, 6, 8, 14 and 15 at 0.1 mM (final conc.) a day after transfection with HER2 antibody. The transfected cells were harvested after 8 days and purified HER2 antibodies were deglycosylated using PNGaseF and the released glycans were purified and analyzed by capillary electrophoresis-laser-induced fluorescence (CE-LIF). The results are shown in Table 1.

TABLE 1

| | Glycan | Control | 16 | 7 | 9 | 13 | 6 | 8 | 14 | 15 |
|---|---|---|---|---|---|---|---|---|---|---|
| Glycan | (alt name) | (%) | (%) | (%) | (%) | (%) | (%) | (%) | (%) | (%) |
| FA2G2S2 | G2FS2 | 0.0 | 0.0 | 0.0 | 0.0 | 0.0 | 0.0 | 0.0 | 0.0 | 3.3 |
| FA2(6)G1S1 | G1FS1 | 0.0 | 5.4 | 0.0 | 0.0 | 0.9 | 0.8 | 0.0 | 0.9 | 17.2 |
| A2 | G0 | 0.0 | 33.2 | 0.0 | 0.0 | 0.0 | 0.0 | 0.0 | 0.0 | 70.6 |
| FA2G2S1 | G2FS1 | 16.8 | 0.0 | 16.6 | 16.4 | 17.0 | 15.9 | 16.8 | 16.4 | 0.0 |
| FA2 | G0F | 78.7 | 57.0 | 79.5 | 78.9 | 77.3 | 77.7 | 79.3 | 76.8 | 8.8 |
| FA2(6)G1 | G1F | 4.6 | 3.6 | 3.9 | 3.9 | 3.9 | 4.1 | 3.9 | 4.4 | 0.0 |
| FA2(3)G1 | G1′F | 0.0 | 0.0 | 0.0 | 0.0 | 0.0 | 0.6 | 0.0 | 0.7 | 0.0 |
| A2BG2 | G2B | 0.0 | 0.8 | 0.0 | 0.8 | 0.8 | 0.8 | 0.0 | 0.8 | 0.0 |

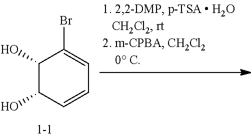

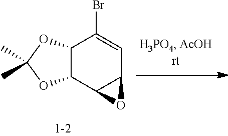

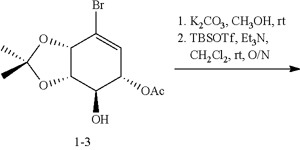

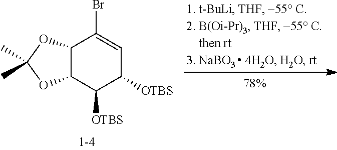

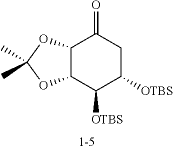

Epoxide 1-2 was prepared according to the literature protocol (Hudlicky, T.; Price, J. D.; Rulin, F.; Tsunoda, T. J. Am. Chem. Soc. 1990, 112, 9439-9440). Acetate 1-3 was prepared according to the literature protocol (Banwell, M. G.; Ma, X.; Karunaratne, O. P.; Willis, A. C. Aust. J. Chem. 2010, 63, 1437-1447).

Synthesis of vinyl bromide 1-4. Potassium carbonate (200 mg, 1.45 mmol) was added to a stirred solution of acetate 1-3 (402 mg, 1.31 mmol) in methanol (5 mL) and stirred for 1 h. The reaction mixture was concentrated under reduced pressure. The solid mass thus obtained was treated with saturated aq NH₄Cl solution (10 mL) and extracted with ethyl acetate (20 mL). The aqueous layer was separated and further extracted with EtOAc (2×20 mL). The combined organic phases were washed with brine (20 mL), dried over anhydrous Na₂SO₄, filtered and concentrated to obtain the crude diol as a pale-yellow viscous liquid. Without further purification, this crude diol was dissolved in CH₂Cl₂ (5 mL) and cooled to 0° C. Triethylamine (1.25 mL, 8.97 mmol) followed by tert-butyldimethylsilyl trifluoromethanesulfonate (0.90 mL, 3.92 mmol) was added dropwise. Ice bath was removed and the reaction mixture was warmed to room temperature. After stirring for 10 h, reaction mixture was treated with saturated aq NH₄Cl solution (10 mL) and extracted with Et₂O (20 mL). The aqueous layer was separated and further extracted with Et₂O (2×20 mL). The combined organic phases were washed with brine (20 mL), dried over anhydrous Na₂SO₄, filtered, concentrated and the resulting crude product was purified by column chromatography (10% Et₂O in hexane) to afford 1-4 (491 mg, 76%) as a white solid: ¹H NMR (500 MHz, CDCl₃) δ 6.06 (d, J=3.4 Hz, 1 H), 4.61 (d, J=6.4 Hz, 1H), 4.16 (t, J=6.2 Hz, 1H), 4.03 (m, 1H), 3.81 (t, J=5.6 Hz, 1H), 1.49 (s, 3H), 1.39 (s, 3H), 0.90 (s, 9H), 0.89 (s, 9H), 0.12 (s, 6H), 0.10 (s, 3H), 0.09 (s, 3H).

Synthesis of ketone 1-5. A flame-dried 50-mL flask equipped with a magnetic stirring bar, an argon inlet, and a septum was charged with acetonide 1-4 (565 mg, 1.14 mmol) and THF (6 mL). The mixture was cooled to −55° C. using dry-ice and acetone bath, then tert-butyllithium (1.45 M in pentane, 1.25 mL, 1.81 mmol) was added dropwise. The reaction mixture was stirred at −55° C. for 30 min, then triisopropylborate (neat, 1.10 mL, 4.77 mmol) was added dropwise. The dry-ice and acetone bath was removed and the reaction mixture was slowly warmed up to room temperature and stirred for 60 h. Then NaBO₃·4H₂O (1.77 g, 11.5 mmol) and H₂O (6 mL) was added. After the mixture had been stirred at rt for 24 h, it was poured into water (20 mL), extracted with Et₂O (30 mL). The aqueous layer was separated and further extracted with Et₂O (2×30 mL). The combined organic phases were washed with brine (20 mL), dried over anhydrous Na₂SO₄, filtered, concentrated and the resulting crude product was purified by column chromatography (20% Et₂O in hexane) to afford 1-5 (386 mg, 78% over the three steps) as a white solid: ¹H NMR (500 MHz, CDCl₃) δ 4.42 (m, 2H), 4.19 (m, 1H), 4.12 (m, 1H), 2.94 (dd, J=13.6, 2.9 Hz, 1H), 2.38 (dd, J=13.6, 3.8 Hz, 1H), 1.41 (s, 3H), 1.36 (s, 3H), 0.92 (s, 9 H), 0.86 (s, 9H), 0.16 (s, 3H), 0.13 (s, 3H), 0.11 (s, 3H), 0.10 (s, 3H); ¹³C NMR (126 MHz, CDCl₃) δ 206.2, 110.6, 82.3, 78.1, 74.6, 70.3, 42.8, 27.3, 25.9, 25.7, 25.6, 18.0, 17.9, −4.88, −4.91, −5.1.

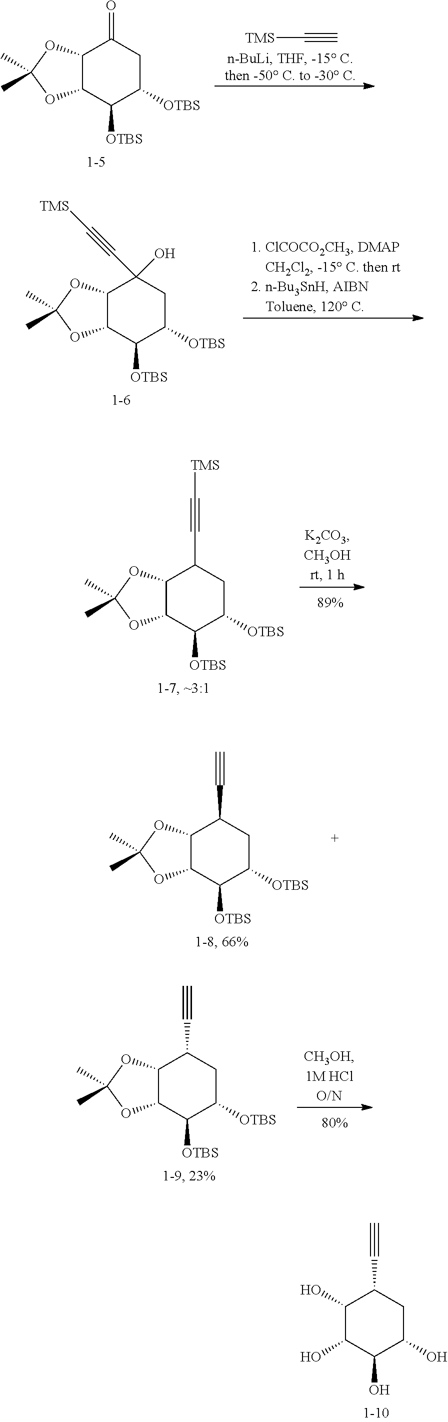

Synthesis of alkyne 1-6. To a stirred solution of ethynyltrimethylsilane (0.25 mL, 1.77 mmol) in THF (1 mL) under Ar atmosphere at −15° C. was added n-butyllithium (2.41 M in hexane, 600 μL, 1.45 mmol). The reaction mixture was allowed to stir at −15° C. for 30 min, and then was cooled to −50° C. Ketone 1-5 (201 mg, 0.467 mmol) in THF (2 mL) was added dropwise to the above reaction mixture and allowed to stir for 3 h between −50° C. and −30° C. Excess lithium trimethylsilylethanide was quenched by the addition of saturated aq NH₄Cl solution (10 mL) and extracted with EtOAc (20 mL). The aqueous layer was separated and further extracted with EtOAc (2×20 mL). The combined organic phases were washed with brine (20 mL), dried over anhydrous Na₂SO₄, filtered, concentrated and the resulting crude product was purified by column chromatography (20% Et₂O in hexane) to afford 1-6 (235 mg, 95%) as a white solid: ¹H NMR (500 MHz, CDCl₃) δ 4.23 (d, J=5.4 Hz, 1H), 3.99 (t, J=5.6 Hz, 1H), 3.77 (m, 1H), 3.65 (dd, J=8.0, 5.9 Hz, 1H), 2.09 (dd, J=13.0, 3.8 Hz, 1H), 2.00 (dd, J=13.0, 9.7 Hz, 1H), 1.54 (s, 3H), 1.37 (s, 3H), 0.91 (s, 9H), 0.90 (s, 9H), 0.18 (s, 9 H), 0.13 (s, 3H), 0.12 (s, 3H), 0.09 (s, 3H), 0.08 (s, 3H); ¹³C NMR (126 MHz, CDCl₃) δ 110.0, 90.8, 81.1, 79.6, 77.8, 77.4, 71.2, 67.7, 27.9, 26.5, 26.4, 26.3, 18.3, 18.2, −0.1, −3.7, −3.7, −4.1, −4.4.

Synthesis of alkyne 1-7. To a stirred solution of alcohol 1-6 (203 mg, 0.384 mmol) in CH₂Cl₂ (3 mL) at 0° C. was added N,N-dimethylaminopyridine (280 mg, 2.29 mmol) followed by methyl 2-chloro-2-oxoacetate (220 μL, 2.39 mmol) in a dropwise manner. Ice bath was removed and the reaction mixture was warmed to room temperature and stirred for 12 h. The reaction mixture was treated with cold saturated aq NaHCO₃ solution (5 mL) and extracted with EtOAc (20 mL). The aqueous layer was separated and further extracted with EtOAc (2×20 mL). The combined organic phases were dried over anhydrous Na₂SO₄, filtered, concentrated and the resulting crude product was quickly purified by column chromatography (15% Et₂O in hexane) to afford the corresponding ester. This ester was dissolved in toluene (1 mL) in a microwavable glass tube. To this reaction mixture was added 2,2′-azobis(2-methylpropionitrile) (75 mg, 0.46 mmol) and the N₂ gas was bubbled through the solution for 30 min. In another microwavable glass tube, neat n-tributyltin hydride (1.20 mL, 4.46 mmol) was added and the N₂ gas was bubbled through the solution for 30 min, after which time the reaction mixture was heated at 120° C. for 10 min. The above crude reaction mixture was added dropwise over 2 min and stirred at 120° C. for 2 h, after which time the solution was cooled to room temperature over 20 min. The reaction mixture was treated with saturated aq NH₄Cl solution (10 mL) and extracted with EtOAc (20 mL). The aqueous layer was separated and further extracted with EtOAc (2×20 mL). The combined organic phases were washed with brine (20 mL), dried over anhydrous Na₂SO₄, filtered, concentrated and the resulting crude product was purified by column chromatography (5% Et₂O in hexane) to afford 1-7 (117 mg, 59% over the two steps) as a colorless viscous liquid which exhibits ˜3:1 diastereomeric ratio, as determined by ¹H NMR spectroscopy in CDCl₃ at 27° C.

Synthesis of alkynes 1-8 and 1-9. Potassium carbonate (18 mg, 0.13 mmol) was added to a stirred solution of alkyne 1-7 (44.9 mg, 87.5 μmol) in CH₃OH (1 mL) and CH₂Cl₂ (0.2 mL) and stirred for 23 h. The reaction mixture was concentrated under reduced pressure. The solid mass thus obtained was treated with saturated aq NH₄Cl solution (5 mL) and extracted with ethyl acetate (10 mL). The aqueous layer was separated and further extracted with EtOAc (2×10 mL). The combined organic phases were washed with brine (10 mL), dried over anhydrous Na₂SO₄, filtered, concentrated and the resulting crude product was purified by column chromatography (5% Et₂O in hexane) to afford pseudo D-altro isomer 1-8 (25.3 mg, 66%) as a colorless viscous liquid and pseudo L-fuco isomer 1-9 (9 mg, 23%) as a colorless viscous liquid. Data for 1-8: ¹H NMR (500 MHz, CDCl₃) δ 4.10 (dd, J=8.5, 5.2 Hz, 1H), 4.00 (dd, J=5.1, 2.7 Hz, 1H), 3.88 (m, 1H), 3.75 (m, 1H), 3.00 (m, 1H), 2.10 (d, J=2.5 Hz, 1H), 1.88 (m, 1H), 1.72 (m, 1H), 1.49 (s, 3H), 1.34 (s, 3H), 0.90 (s, 9H), 0.89 (s, 9H), 0.10 (s, 3H), 0.08 (s, 6H), 0.07 (s, 3H); ¹³C NMR (126 MHz, CDCl₃) δ 108.9, 86.6, 78.5, 77.5, 72.1, 69.7, 69.1, 32.2, 28.5, 27.1, 26.2, 25.9, 18.1, −4.5, −4.6, −4.7, −4.8. Data for 1-9: ¹H NMR (500 MHz, CDCl₃) δ 4.30 (m, 1H), 3.98 (m, 1H), 3.61 (m, 1H), 3.51 (m, 1H), 2.82 (m, 1H), 2.17 (d, J=2.5 Hz, 1H), 1.97-1.85 (m, 2H), 1.54 (s, 3H), 1.38 (s, 3H), 0.89 (s, 9H), 0.88 (s, 9H), 0.11 (s, 3H), 0.08 (s, 6H), 0.07 (s, 3H); ¹³C NMR (126 MHz, CDCl₃) δ 109.5, 83.9, 80.4, 77.2, 74.9, 72.7, 69.8, 33.2, 27.6, 27.5, 26.2, 26.1, 25.8, 18.2, −3.8, −3.9, −4.2, −4.3.

Synthesis of tetraol 1-10. To a stirred solution of alkyne 1-9 (15 mg, 34 μmol) in CH₃OH (1 mL) was added 1 M HCl (0.4 mL, 0.4 mmol) and stirred for 14 h. The reaction mixture was concentrated under reduced pressure and co-evaporated with toluene (3×2 mL) and the resulting crude product was purified by column chromatography (5% CH₃OH in EtOAc) to afford 1-10 (4.7 mg, 80%) as a colorless viscous liquid: ¹H NMR (500 MHz, CD₃OD) δ 3.93 (m, 1H), 3.49 (m, 1H), 3.35 (m, 1H), 3.25 (dd, J=9.6, 3.0 Hz, 1H), 2.55 (m, 1H), 2.37 (d, J=2.5 Hz, 1H), 1.93-1.83 (m, 2H); ¹³C NMR (126 MHz, CD₃OD) δ 85.2, 76.0, 75.5, 73.0, 72.9, 70.7, 34.1, 31.8; HRMS (ESI-TOF) m/z: [M+NH₄]⁺ Calcd for C₈H₁₂O₄ 190.1074; Found 190.1074.

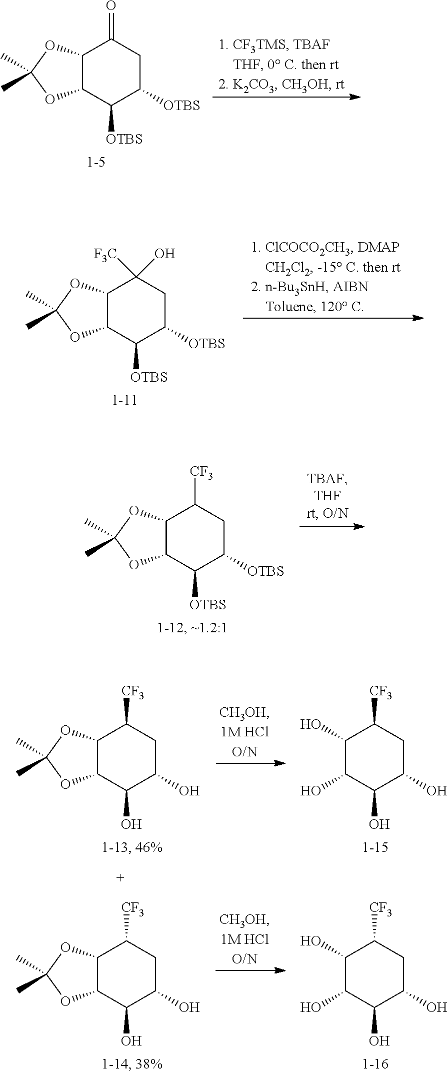

Synthesis of trifluoromethyl compound 1-11. To a stirred solution of ketone 1-5 (141 mg, 0.327 mmol) in THF (2 mL) at 0° C. was added trimethyl(trifluoromethyl)silane (2.00 M in THF, 180 μL, 360 μmol) followed by tetra-n-butylammonium fluoride (1.0 M in THF, 10 μL, 10 μmol). Ice bath was removed and the reaction mixture was warmed to room temperature and stirred for 30 min. The reaction mixture was treated with saturated aq NH₄Cl solution (5 mL) and extracted with EtOAc (20 mL). The organic layer was dried over anhydrous Na₂SO₄, filtered, concentrated and the resulting crude material was dissolved in CH₃OH (2 mL). Potassium carbonate (50 mg, 0.36 mmol) was added to the above solution and stirred for 1 h. The reaction mixture was concentrated under reduced pressure. The solid mass thus obtained was treated with saturated aq NH₄Cl solution (5 mL) and extracted with ethyl acetate (20 mL). The aqueous layer was separated and further extracted with EtOAc (2×20 mL). The combined organic phases were washed with brine (10 mL), dried over anhydrous Na₂SO₄, filtered, concentrated and the resulting crude product was purified by column chromatography (10% Et₂O in hexane) to afford 1-11 (110 mg, 67%) as a colorless viscous liquid: ¹H NMR (500 MHz, CDCl₃) δ 4.3 (d, J=6.4 Hz, 1H), 4.17-4.11 (m, 2H), 3.92 (m, 1H), 2.00 (m, 2H), 1.56 (s, 3 H), 1.38 (s, 1H), 0.91 (s, 9H), 0.88 (s, 9H), 0.12 (s, 12H).

Synthesis of trifluoromethyl compounds 1-12. To a stirred solution of alcohol 1-11 (352 mg, 0.703 mmol) in CH₂Cl₂ (3 mL) at 0° C. was added N,N-dimethylaminopyridine (515 mg, 4.22 mmol) followed by methyl 2-chloro-2-oxoacetate (0.40 mL, 4.4 mmol) in a dropwise manner. Ice bath was removed and the reaction mixture was warmed to room temperature and stirred for 14 h. The reaction mixture was treated with cold saturated aq NaHCO₃ solution (5 mL) and extracted with EtOAc (30 mL). The aqueous layer was separated and the organic phase was dried over anhydrous Na₂SO₄, filtered, concentrated and the resulting crude product was quickly purified by column chromatography (10% Et₂O in hexane) to afford the corresponding ester along with the hydrolysed ester (starting material). This crude material was dissolved in toluene (1 mL) in a microwavable glass tube. To this reaction mixture was added 2,2′-azobis(2-methylpropionitrile) (60 mg, 0.37 mmol) and the N₂ gas was bubbled through the solution for 30 min. In an another microwavable glass tube, neat n-tributyltin hydride (900 μL, 3.35 mmol) was added and the N₂ gas was bubbled through the solution for 30 min, after which time the reaction mixture was heated at 120° C. for 10 min. The above crude reaction mixture was added dropwise over 2 min and stirred at 120° C. for 2 h, after which time the solution was cooled to room temperature over 20 min. The reaction mixture was treated with saturated aq NH₄Cl solution (10 mL) and extracted with EtOAc (30 mL). The aqueous layer was separated and further extracted with EtOAc (2×20 mL). The combined organic phases were washed with brine (20 mL), dried over anhydrous Na₂SO₄, filtered, concentrated and the resulting crude product was purified by column chromatography (2.5% Et₂O in hexane) to afford 1-12 (119 mg, 35% over the two steps) as a colorless viscous liquid which exhibits ˜1.2:1 diastereomeric ratio, as determined by ¹H NMR spectroscopy in CDCl₃ at 27° C. along with 1-11 (65 mg, 18%).

Synthesis of diols 1-13 and 1-14. To a stirred solution of acetonide 1-12 (100 mg, 0.206 mmol) in THF (2 mL) was added tetra-n-butylammonium fluoride (1.0 M in THF, 1.0 mL, 1.0 mmol) and stirred for 10 h. The reaction mixture was then treated with H₂O and extracted with ethyl acetate (20 mL). The aqueous layer was separated and further extracted with EtOAc (2×20 mL). The combined organic phases were washed with brine (10 mL), dried over anhydrous Na₂SO₄, filtered, concentrated and the resulting crude product was purified by column chromatography (65% EtOAc in hexane) to afford 1-13 (24 mg, 46%) as a colorless viscous liquid and 1-14 (80% EtOAc in hexane, 20 mg, 38%) as a white solid. Data for 1-13: ¹H NMR (500 MHz, CDCl₃) δ 4.35 (t, J=5.3 Hz, 1H), 4.03 (t, J=6.8 Hz, 1 H), 3.78 (m, 1H), 3.67 (t, J=7.7 Hz, 1H), 2.81 (m, 1H), 2.04 (m, 1H), 1.88 (m, 1 H), 1.53 (s, 3H), 1.38 (s, 3H); ¹³C NMR (126 MHz, CDCl₃): δ 126.9, 109.8, 78.6, 75.6, 72.2, 68.0, 40.2, 28.1, 26.2, 25.9. Data for 1-14: ¹H NMR (500 MHz, CDCl₃) δ 4.37 (m, 1H), 3.95 (m, 1H), 3.56 (m, 1H), 3.48 (m, 1H), 2.54 (m, 1H), 2.06 (m, 1 H), 1.85 (m, 1H), 1.55 (s, 3H), 1.38 (s, 3H); ¹³C NMR (126 MHz, CDCl₃) δ 127.0, 110.9, 80.5, 77.8, 71.5, 70.4, 40.1, 28.4, 26.4, 25.9.

Synthesis of tetraol 1-15. To a stirred solution of diol 1-13 (10.1 mg, 39.4 μmol) in CH₃OH (0.9 mL) was added 1 M HCl (0.2 mL, 0.2 mmol) and stirred for 12 h. The reaction mixture was concentrated under reduced pressure and co-evaporated with toluene (3×2 mL) and the resulting crude product was purified by column chromatography (3% CH₃OH in EtOAc) to afford 1-15 (6.1 mg, 72%) as a colorless viscous liquid: ¹H NMR (500 MHz, CD₃OD) δ 4.02 (dd, J=7.1, 3.1 Hz, 1H), 3.80-3.72 (m, 2H), 3.70 (m, 1H), 2.72 (m, 1H), 1.99-1.88 (m, 2H); ¹³C NMR (126 MHz, CD₃OD) δ 129.0, 74.7, 73.4, 70.6, 68.2, 41.8, 27.7; ¹⁹F NMR (377 MHz, CD₃OD) δ −68.7; HRMS (ESI-TOF) m/z: [M+NH₄]⁺ Calcd for C₇H₁₁F₃O₄ 234.0948; Found 234.0951.

Synthesis of tetraol 1-16. To a stirred solution of diol 1-14 (7.40 mg, 28.9 μmol) in CH₃OH (0.9 mL) was added 1 M HCl (0.2 mL, 0.2 mmol) and stirred for 12 h. The reaction mixture was concentrated under reduced pressure and co-evaporated with toluene (3×2 mL) and the resulting crude product was purified by column chromatography (3% CH₃OH in EtOAc) to afford 1-16 (5.1 mg, 82%) as a colorless viscous liquid: ¹H NMR (500 MHz, CD₃OD) δ 4.13 (m, 1H), 3.53 (m, 1H), 3.43 (m, 1H), 3.28 (dd, J=9.6, 3.0 Hz, 1H), 2.35 (m, 1H), 1.93-1.82 (m, 2H); ¹³C NMR (126 MHz, CD₃OD) δ 128.2, 76.1, 75.4, 72.5, 68.8, 42.7, 27.2; HRMS (ESI-TOF) m/z: [M+NH₄]⁺ Calcd for C₇H₁₁F₃O₄ 234.0948; Found 234.0952.

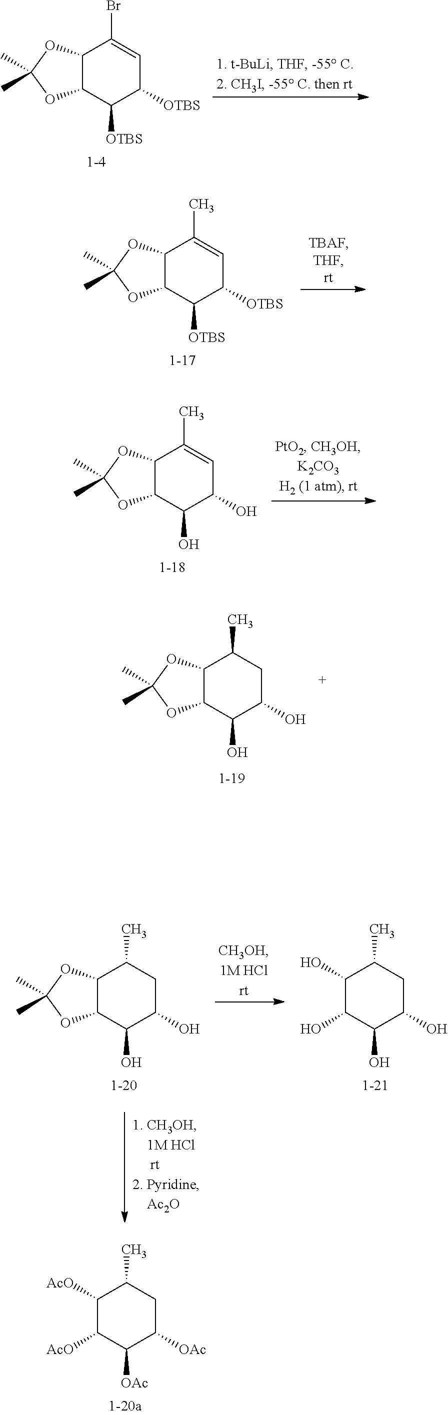

Synthesis of alkene 1-17. A flame-dried 50-mL flask equipped with a magnetic stirring bar, an argon inlet, and a septum was charged with acetonide 1-4 (199 mg, 0.403 mmol) and THF (3 mL). The mixture was cooled to −55° C. using dry-ice and acetone bath, then tert-butyllithium (1.55 M in pentane, 410 μL, 0.636 mmol) was added dropwise. The reaction mixture was stirred at −55° C. for 30 min, then methyl iodide (0.10 mL, 1.6 mmol) was added dropwise. The dry-ice and acetone bath was removed and the reaction mixture was slowly warmed up to room temperature and stirred for 2 h. The reaction mixture was treated with saturated aq NH₄Cl solution (10 mL) and extracted with Et₂O (30 mL). The aqueous layer was separated and further extracted with Et₂O (2×20 mL). The combined organic phases were washed with brine (20 mL), dried over anhydrous Na₂SO₄, filtered, concentrated and the resulting crude product was purified by column chromatography (2% Et₂O in hexane) to afford 1-17 (104 mg, 60% over the two steps) as a colorless viscuous liquid: ¹H NMR (500 MHz, CDCl₃) δ 5.36 (m, 1H), 4.42 (d, J=7.0 Hz, 1H), 4.02 (m, 1H), 3.99 (m, 1H), 3.56 (m, 1H), 1.80 (s, 3H), 1.45 (s, 3H), 1.35 (s, 3H), 0.91 (s, 9 H), 0.90 (s, 9H), 0.13 (s, 3H), 0.09 (s, 6H), 0.08 (s, 3H); ¹³C NMR (126 MHz, CDCl₃) δ 131.6, 129.0, 109.5, 78.8, 76.1, 75.8, 71.7, 28.0, 26.3, 26.2, 25.8, 19.9, 18.4, 18.3, −3.5, −3.7, −3.9, −4.4.

Synthesis of diol 1-18. To a stirred solution of acetonide 1-17 (119.5 mg, 0.2787 mmol) in THF (2 mL) was added tetra-n-butylammonium fluoride (1.0 M in THF, 1.4 mL, 1.4 mmol) and stirred for 10 h. The reaction mixture was then treated with H₂O and extracted with ethyl acetate (20 mL). The aqueous layer was separated and further extracted with EtOAc (2×20 mL). The combined organic phases were washed with brine (10 mL), dried over anhydrous Na₂SO₄, filtered, concentrated and the resulting crude product was purified by column chromatography (95% EtOAc in hexane) to afford 1-18 (50 mg, 90%) as a colorless viscous liquid: ¹H NMR (500 MHz, CDCl₃) δ 5.50 (s, 1H), 4.42 (d, J=6.5 Hz, 1H), 4.07 (dd, J=8.9, 6.6 Hz, 1H), 4.03 (m, 1H), 3.52 (t, J=8.5 Hz, 1H), 1.82 (s, 3H), 1.48 (s, 3H), 1.37 (s, 3H); ¹³C NMR (126 MHz, CDCl₃) δ 131.9, 128.0, 110.3, 77.7, 76.1, 75.4, 70.5, 28.3, 25.9, 20.1.

Synthesis of diol 1-20. A solution of diol 1-18 (216 mg, 1.08 mmol) in CH₃OH (6 mL) was stirred under an argon balloon. To this solution was added potassium carbonate (15 mg, 0.11 mmol) followed by PtO₂ (26 wt %, 56 mg, 0.25 mmol) under an argon atmosphere. The argon balloon was replaced with a H₂ balloon (1 atm) and the solution was flushed with H₂ for 10 sec. The reaction mixture was stirred under H₂ balloon for 24 h and then the H₂ balloon was replaced with argon balloon and flushed with argon for 1 min. The reaction mixture was diluted with CH₃OH (30 mL) and filtered over a pad of Celite-545. The filtrate was concentrated and the resulting crude product was purified by column chromatography (95% EtOAc in hexane) to afford 1-20 (126 mg, 58%) as a colorless viscuous liquid along with D-altro isomer 1-19 (26 mg, 12%). Data for 1-20: ¹H NMR (500 MHz, CDCl₃) δ 4.07 (m, 1H), 3.86 (m, 1H), 3.48 (m, 1H), 3.44 (m, 1H), 1.92 (m, 1H), 1.80-1.69 (m, 2H), 1.51 (s, 3H), 1.35 (s, 3H), 1.12 (d, J=6.9 Hz, 3H); ¹³C NMR (126 MHz, CDCl₃) δ 109.2, 81.0, 78.6, 78.3, 71.4, 34.5, 30.0, 28.6, 26.5, 17.4; HRMS (ESI-TOF) m/z: [M+NH₄]⁺ Calcd for C₁₀H₁₈O₄ 203.1278; Found 203.1275.

Synthesis of carbafucose 1-21. To a stirred solution of diol 1-20 (62.0 mg, 0.307 mmol) in CH₃OH (8.3 mL) was added 1 M HCl (1.7 mL, 1.7 mmol) and stirred for 12 h. The reaction mixture was concentrated under reduced pressure and co-evaporated with toluene (3×3 mL) and the resulting crude product was purified by column chromatography (5% CH₃OH in EtOAc) to afford 1-21 (42 mg, 85%) as a white solid: [α]_{D}²⁰=+22.3 (c=0.05, CH₃OH); ¹H NMR (600 MHz, CD₃OD) δ 3.68 (brs, 1H), 3.48-3.45 (m, 1H), 3.39-3.35 (m, 1H), 3.27 (dd, J=9.6, 3.0 Hz, 1 H), 1.65-1.60 (m, 1H), 1.57-1.48 (m, 2H), 1.02 (d, J=6.8 Hz, 3H); ¹³C NMR (151 MHz, CD₃OD): δ 76.5, 76.4, 74.9, 73.8, 35.8, 33.1, 17.8; IR (neat): v 3368, 2958, 2928, 2857, 1731, 1668, 1462, 1261, 1067, 1022, 799 cm⁻¹; HRMS (ESI-TOF) m/z: [M+Na]⁺ Calcd for C₇H₁₄NaO₄ 185.0784; Found 185.0784.

Synthesis of acetate 1-20a. To a stirred solution of diol 1-20 (6 mg, 29 μmol) in CH₃OH (1 mL) was added 1 M HCl (0.2 mL, 0.4 mmol) and stirred for 12 h. The reaction mixture was concentrated under reduced pressure and co-evaporated with toluene (3×2 mL) and the resulting crude product was dissolved in pyridine (1 mL). Ac₂O (0.10 mL, 1.1 μmol) was added and heated at 50° C. for 12 h. The reaction mixture was concentrated under reduced pressure and co-evaporated with toluene (3×2 mL) and the resulting crude product was purified by column chromatography (20% EtOAc in hexane) to afford 1-20a (7.8 mg, 80% over the two steps) as a white solid: ¹H NMR (500 MHz, CDCl₃) δ 5.36 (t, J=10.1 Hz, 1H), 5.31 (m, 1H), 4.92-4.86 (m, 2H), 2.14 (s, 3H), 2.03 (s, 3H), 2.02 (s, 3H), 1.97 (s, 3H), 1.92 (m, 1H), 1.89 (m, 1H), 1.64 (m, 1H); HRMS (ESI-TOF) m/z: [M+NH₄]⁺ Calcd for C₁₅H₂₆NO₈ 348.1653; Found 348.1650.

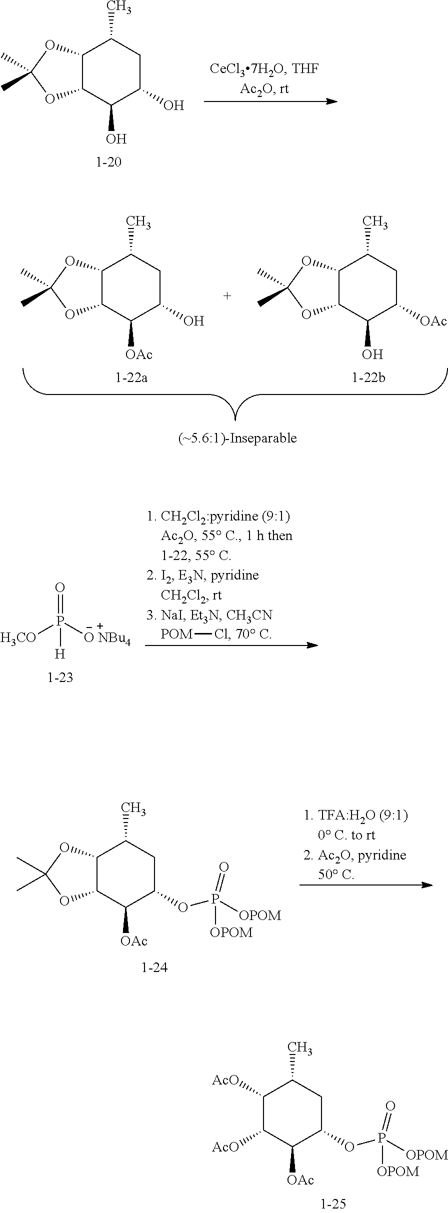

Synthesis of acetates 1-22a and 1-22b. To a stirred solution of diol 1-20 (63.5 mg, 0.314 mmol) in THF (1.5 mL) was added CeCl₃·7H₂O (11 mg, 30 μmol) followed by acetic anhydride (150 μL, 1.59 mmol) and stirred for 24 h. The reaction mixture was treated with saturated aq NaHCO₃ solution (10 mL) and extracted with EtOAc (20 mL). The aqueous layer was separated and further extracted with EtOAc (2×20 mL). The combined organic phases were washed with brine (20 mL), dried over anhydrous Na₂SO₄, filtered, concentrated and the resulting crude product was purified by column chromatography (40% EtOAc in hexane) to afford inseparable 1-22a and 122b (60 mg, 78%) as a colorless viscous liquid which exhibits ˜5.6:1 regioisomeric ratio, as determined by ¹H NMR spectroscopy in CDCl₃ at 27° C.: ¹H NMR (400 MHz, CDCl₃, Major isomer) 64.88 (dd, J=10.1, 7.7 Hz, 1H), 4.08 (m, 1H), 3.98 (dd, J=7.7, 4.8 Hz, 1H), 3.48 (m, 1H), 2.14 (s, 3H), 1.91 (m, 1 H), 1.81 (m, 1H), 1.61 (m, 1H), 1.53 (s, 3H), 1.34 (s, 3H), 1.13 (d, J=7.0 Hz, 3H); ¹³C NMR (101 MHz, CDCl₃) δ 172.2, 109.5, 79.8, 78.4, 78.1, 70.9, 35.8, 29.8, 28.1, 26.4, 21.3, 17.3; HRMS (ESI-TOF) m/z: [M+NH₄]⁺ Calcd for C₁₂H₂₀O₅ 245.1384; Found 245.1387.

Synthesis of phosphate 1-24. To a stirred solution of tetra-n-butylammonium methyl H-phosphonate 1-23 (125 mg, 0.389 mmol) in CH₂Cl₂ (2.7 mL) and pyridine (0.3 mL) was added Ac₂O (40 μL, 0.42 mmol). The reaction mixture was stirred at 55° C. for 1 h before being cooled to room temperature over 5 min using an ice bath. A solution of acetates 1-22 (12.0 mg, 37.3 μmmol) in CH₂Cl₂ (1.5 mL) was added dropwise to the above cooled reaction mixture over 5 min and the reaction mixture was stirred at 55° C. for 15 h before being cooled to room temperature over 15 min. The reaction mixture was concentrated and the resulting crude product was quickly purified by column chromatography (75% EtOAc in hexane) to obtain the desired 2-O-acetyl H-phosphonate. This H-phosphonate (8.0 mg, 24.8 μmmol) was dissolved in CH₂Cl₂ (1 mL) and cooled to 0° C. Et₃N (20 μL, 0.18 mmol) followed by I₂ (16 mg, 63 μmmol) in pyridine (1 mL) were added. The ice bath was removed and the reaction mixture was warmed to room temperature over 4 h. Excess I₂ was quenched by the addition of saturated aq Na₂S₂O₃ solution (5 mL) and the reaction mixture was extracted with EtOAc (20 mL). The aqueous layer was separated and further extracted with EtOAc (2×20 mL). The combined organic phases were washed with brine (10 mL), dried over anhydrous Na₂SO₄, filtered, concentrated and the resulting crude product was quickly purified by column chromatography (20% CH₃OH in EtOAc) to obtain the desired methyl H-phosphonate. This was dissolved in CH₃CN (1.5 mL). Et₃N (35 μL, 0.25 mmol), Nal (18 mg, 0.12 mmol) followed by POM-Cl (35 μL, 0.24 mmol) were added sequentially and was heated at 70° C. for 14 h. The reaction mixture was treated with saturated aq Na₂S₂O₃ solution (10 mL) and extracted with EtOAc (20 mL). The aqueous layer was separated and further extracted with EtOAc (2×20 mL). The combined organic phases were washed with brine (20 mL), dried over anhydrous Na₂SO₄, filtered, concentrated and the resulting crude product was purified by column chromatography (30% EtOAc in hexane) to afford 1-24 (2.7 mg, 10% over the three steps) as a colorless viscuous liquid: ¹H NMR (400 MHz, CDCl₃) δ 5.66-5.56 (m, 4 H), 5.12 (dd, J=10.3, 7.9 Hz, 1H), 4.28 (m, 1H), 4.08 (m, 1H), 3.98 (dd, J=7.7, 4.8 Hz, 1H), 2.12 (s, 3H), 2.07-1.91 (m, 3H), 1.55 (s, 3H), 1.35 (s, 3H), 1.24 (s, 18 H), 1.15 (d, J=6.7 Hz, 3H); ³¹P NMR (162 MHz, CDCl₃) δ −5.08; HRMS (ESI-TOF) m/z: [M+NH₄]⁺ Calcd for C₂₄H₄₁O₁₂P 570.2674; Found 570.2675.

Synthesis of 1-25. A cooled solution of TFA:H₂O (9:1, 0.5 mL) was added to the acetonide 1-24 (2.0 mg, 3.6 μmol) and stirred for 3 h. The reaction mixture was concentrated under reduced pressure and co-evaporated with toluene (3×2 mL) and the resulting crude product was dissolved in pyridine (1 mL). Ac₂O (0.10 mL, 1.1 μmol) was added and heated at 50° C. for 24 h. The reaction mixture was concentrated under reduced pressure and co-evaporated with toluene (3×2 mL) and the resulting crude product was purified by column chromatography (30% EtOAc in hexane) to afford 1-25 (1.5 mg, 69% over the two steps) as a colorless viscuous liquid: ¹H NMR (400 MHz, CDCl₃) δ 5.66-5.58 (m, 4H), 5.36-5.27 (m, 2H), 4.83 (dd, J=10.5, 3.0 Hz, 1H), 4.41 (m, 1H), 2.14 (s, 3H), 2.08 (s, 3H), 2.06 (m, 1H), 1.97 (s, 3H), 1.92-1.78 (m, 2H), 1.24 (s, 18H), 0.95 (d, J=6.6 Hz, 3H); ³¹P NMR (162 MHz, CDCl₃) δ −5.11. HRMS (ESI-TOF) m/z: [M+NH₄]⁺ Calcd for C₂₅H₄₁O₁₄P 614.2572; Found 614.2575.

### Phosphoric Acid

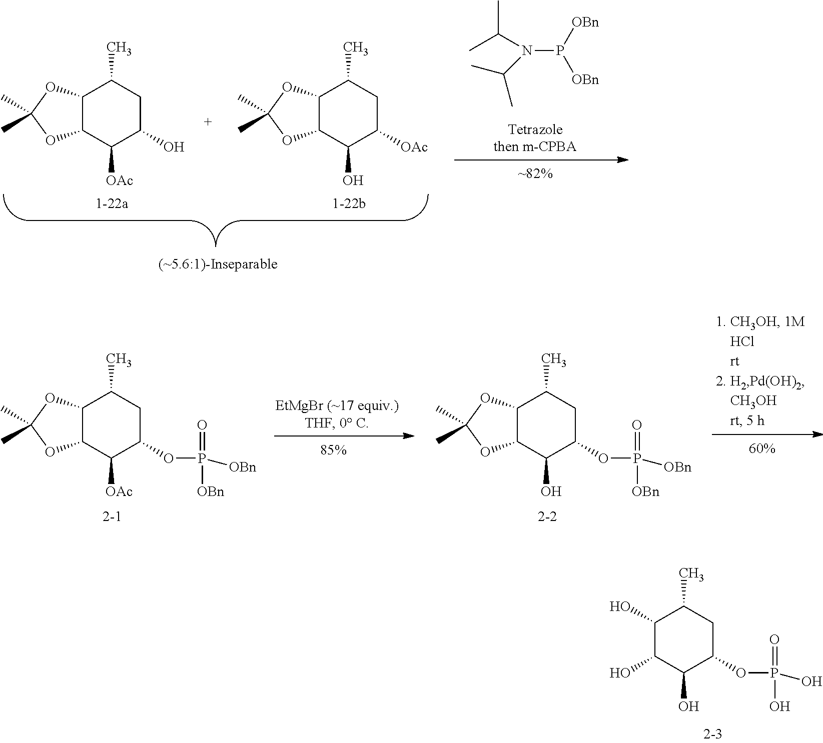

Synthesis of dibenzyl phosphate 2-1. To a stirred solution of acetates 1-22a and 1-22b (233 mg, 0.954 mmol) in CH₂Cl₂ (4 mL) was added 1 H tetrazole (0.40 in acetonitrile, 6.0 mL, 2.4 mmol) followed by dibenzyl diisopropylphosphoramidite (0.80 mL, 2.4 mmol) at rt. After 1 hour, m-CPBA (77%, 854 mg, 3.81 mmol) was added to the mixture at 0° C. After being stirred 1 hour at 0° C., the mixture was diluted with CH₂Cl₂ (30 mL) and washed with saturated aq Na₂SO₃ (10 mL), saturated aq NaHCO₃ (10 mL). The aqueous layer was separated and further extracted with CH₂Cl₂ (2×20 mL). The combined organic phases were washed with brine (20 mL), dried over anhydrous Na₂SO₄, filtered, concentrated and the resulting crude product was purified by column chromatography (25% EtOAc in hexane) to afford 2-1 (337 mg, 70%) as a white solid along with the undesired isomer (12%): Data for 2-1: ¹H NMR (500 MHz, CDCl₃) δ 7.38-7.28 (m, 10H), 5.13 (dd, J=10.2, 7.7 Hz, 1H), 5.03-4.92 (m, 4H), 4.24 (dddd, J=11.4, 10.2, 7.5, 4.0 Hz, 1H), 4.09-4.03 (m, 1H), 3.96 (dd, J=7.8, 4.7 Hz, 1H), 1.92 (s, 3H), 1.91-1.82 (m, 2H), 1.77-1.66 (m, 1H), 1.56 (s, 3H), 1.34 (s, 3H), 1.10 (d, J=6.7 Hz, 3H); ¹³C NMR (125 MHz, CDCl₃) δ 170.5, 135.8 (2C), 128.7, 128.7, 128.1, 128.0, 109.8, 78.4, 77.7, 76.3, 76.3, 75.8, 75.7, 69.5, 69.4, 69.4, 33.7, 29.4, 28.0, 26.5, 21.1, 17.1; ³¹P NMR (162 MHz, CDCl₃) 5-1.79.

Synthesis of acetonide 2-2. To a cooled (0° C.) solution of acetate 2-1 (85 mg, 0.17 mmol) in THF (3 mL) was added EtMgBr (3.0 M in Et₂O, 0.95 mL, 2.9 mmol) and stirred at 0° C. for 1.5 h. Excess Grignard reagent was quenched by the addition of saturated aq NH₄Cl solution (10 mL) and extracted with EtOAc (20 mL). The aqueous layer was separated and further extracted with EtOAc (2×20 mL). The combined organic phases were washed with brine (15 mL), dried over anhydrous Na₂SO₄, filtered, concentrated and the resulting crude product was purified by column chromatography (45% EtOAc in hexane) to afford 2-2 (65 mg, 85%) as a colorless viscous liquid: ¹H NMR (600 MHz, CDCl₃) δ 7.41-7.28 (m, 11H), 5.13-4.90 (m, 4H), 4.11-3.98 (m, 2H), 3.87 (dd, J=7.3, 4.9 Hz, 1H), 3.61 (dd, J=9.7, 7.3 Hz, 1H), 1.82 (ddq, J=16.2, 6.9, 3.5 Hz, 1H), 1.72 (dt, J=12.7, 4.0 Hz, 1H), 1.66-1.54 (m, 1H), 1.50 (s, 3H), 1.34 (s, 3H), 1.08 (d, J=6.9 Hz, 3H); ¹³C NMR (151 MHz, CDCl₃) δ 135.81, 135.80, 135.76, 135.75, 128.8, 128.74, 128.72, 128.2, 128.18, 128.15, 128.13, 109.4, 80.7, 79.7, 79.66, 77.5, 76.5, 76.4, 69.81, 69.77, 69.72, 69.68, 33.22, 33.20, 29.6, 28.5, 26.4, 17.2; ³¹P NMR (162 MHz, CDCl₃) δ −0.82.

Synthesis of 2-3. To a stirred solution of acetonide 2-2 (30.3 mg, 65.5 μmol) in CH₃OH (1.2 mL) was added 1 M HCl (0.3 mL, 0.3 mmol) and stirred for 2 h. The reaction mixture was concentrated under reduced pressure and co-evaporated with toluene (3×2 mL) and the resulting crude product was quickly purified by column chromatography (5% CH₃OH in EtOAc) to afford the corresponding triol (22.7 mg, 82%) as a colorless viscous liquid. A solution of this triol (22.7 mg, 53.7 μmol) in CH₃OH (3 mL) was stirred under an argon. Pd(OH)₂—C (20 wt %, 17 mg, 0.024 mmol) were added to the reaction mixture and then the argon balloon was replaced with a H₂ balloon (1 atm) and stirred for 5 h. At this point, the H₂ balloon was replaced with argon balloon and flushed argon for 1 min. The reaction mixture was diluted with CH₃OH (20 mL) and the solution was filtered over a pad of Celite-545. The filtrate was concentrated, and the resulting crude product was purified by column chromatography (50% CH₃OH in CH₂Cl₂) on spherical silica gel (40-75 μm particle size) to afford 2-3 (9.5 mg, 60% over the two steps) as a white solid: ¹H NMR (500 MHz, D₂O) δ 3.98-3.91 (m, 1H), 3.79 (t, J=2.8 Hz, 1H), 3.60 (dd, J=9.5, 9.5 Hz, 1 H), 3.49 (dd, J=10.0, 3.1 Hz, 1H), 1.89 (dt, J=13.0, 4.2 Hz, 1H), 1.78-1.69 (m, 1 H), 1.48 (td, J=13.0, 11.5 Hz, 1H), 0.99 (d, J=6.9 Hz, 3H).

### POM-Phosphate Triol

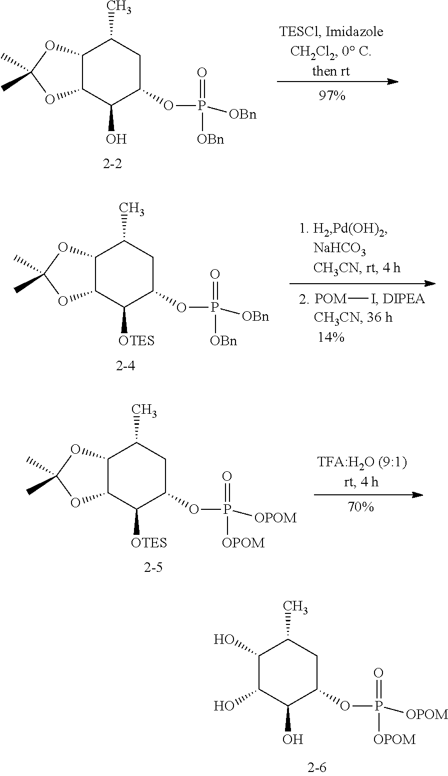

Synthesis of dibenzyl phosphate 2-4. To a cooled (0° C.) solution of alcohol 2-2 (37 mg, 80 μmol) in CH₂Cl₂ (2 mL) was added imidazole (55 mg, 0.81 mmol) followed by TESCl (0.10 mL, 0.74 mmol). Ice-bath was removed and stirred at rt for 2 h. Reaction mixture is diluted with CH₂Cl₂ and washed with saturated aq NaHCO₃ solution (10 mL) and extracted with CH₂Cl₂ (20 mL). The aqueous layer was separated and further extracted with CH₂Cl₂ (2×20 mL). The combined organic phases were washed with brine (15 mL), dried over anhydrous Na₂SO₄, filtered, concentrated and the resulting crude product was purified by column chromatography (20% EtOAc in hexane) to afford 2-4 (44.6 mg, 97%) as a colorless viscous liquid: ¹H NMR (400 MHz, CDCl₃) δ 7.38-7.28 (m, 10H), 5.14-4.94 (m, 4H), 4.13-3.96 (m, 2H), 3.82 (dd, J=6.6, 5.1 Hz, 1H), 3.63 (dd, J=9.1, 6.6 Hz, 1H), 1.95 (dt, J=12.6, 4.0 Hz, 1H), 1.89-1.77 (m, 1H), 1.68-1.53 (m, 1H), 1.49 (s, 3H), 1.33 (s, 3H), 1.05 (d, J=6.9 Hz, 3H), 0.93 (t, J=7.9 Hz, 9H), 0.69-0.58 (m, 6H); ³¹P NMR (162 MHz, CDCl₃) δ −1.49.

Synthesis of POM-phosphate 2-5. To a stirred solution of dibenzyl phosphate 2-4 (46 mg, 80 μmol) in CH₃CN (3 mL) was added NaHCO₃ (76 mg, 0.90 mmol) followed by Pd(OH)₂—C (20 wt %, 23 mg, 33 μmol) were added to the reaction mixture under argon. The argon balloon was replaced with a H₂ balloon (1 atm) and stirred for 4 h. At this point, the H₂ balloon was replaced with argon balloon and flushed argon for 1 min. The reaction mixture was diluted with CH₃OH (20 mL) and the solution was filtered over a pad of Celite-545. The filtrate was concentrated, and the resulting crude product was dissolved in CH₃CN (2 mL) and CH₂Cl₂ (0.5 mL). DIPEA (0.14 mL, 0.80 mmol) followed by POM-I (0.15 mL, 0.99 mmol) were added sequencially and stirred for 36 h. The reaction mixture was treated with saturated aq Na₂S₂O₃ solution (10 mL) and extracted with EtOAc (20 mL). The aqueous layer was separated and further extracted with EtOAc (2×20 mL). The combined organic phases were washed with brine (15 mL), dried over anhydrous Na₂SO₄, filtered, concentrated and the resulting crude product was purified by column chromatography (13% EtOAc in hexane) to afford 2-5 (7 mg, 14% over the two steps) as a pale yellow viscuous liquid: ¹H NMR (600 MHz, CDCl₃) δ 5.71-5.57 (m, 4H), 4.15-4.05 (m, 1H), 4.04-4.00 (m, 1H), 3.87-3.75 (m, 1H), 3.61 (dd, J=9.1, 6.7 Hz, 1H), 1.99 (dt, J=12.9, 4.2 Hz, 1H), 1.94-1.82 (m, 1H), 1.70-1.57 (m, 1H), 1.47 (s, 3H), 1.33 (s, 3H), 1.23 (s, 18H), 1.10 (d, J=7.0 Hz, 3H), 0.94 (t, J=8.0 Hz, 9H), 0.70-0.57 (m, 6H); ¹³C NMR (150 MHz, CDCl₃) δ 176.8 (2C), 109.0, 83.0, 82.9, 82.81, 82.77, 81.9, 81.8, 80.3, 80.2, 77.8, 77.2, 77.1, 38.9, 33.0, 29.5, 28.4, 26.99, 26.97, 26.5, 17.3, 6.9 (3C), 5.0 (3C); ³¹P NMR (162 MHz, CDCl₃) 5-4.67.

Synthesis of POM-phosphate triol 2-6. A solution of TFA:H₂O (9:1, 0.7 mL) was added to the acetonide 2-6 (7.6 mg, 12 μmol) and stirred for 4 h. The reaction mixture was concentrated under reduced pressure and co-evaporated with toluene (3×2 mL) and the resulting crude product was purified by column chromatography (90% EtOAc in hexane) to afford triol 2-6 (4.0 mg, 70%) as a pale yellow viscuous liquid: ¹H NMR (600 MHz, CDCl₃) δ 5.75-5.57 (m, 4H), 4.33-4.19 (m, 1H), 3.86-3.83 (m, 1H), 3.80 (app t, J=9.4 Hz, 1H), 3.46 (dd, J=9.4, 2.9 Hz, 1 H), 1.90-1.73 (m, 2H), 1.72-1.62 (m, 1H), 1.24 (s, 18H), 1.08 (d, J=6.8 Hz, 3H); ¹³C NMR (150 MHz, CDCl₃) δ 177.4, 177.0, 83.0, 82.99, 82.97, 82.95, 81.4, 81.3, 74.8, 73.63, 73.60, 71.9, 38.94, 38.91, 32.93, 32.91, 31.2, 29.9, 26.97, 26.96, 17.1; ³¹P NMR (162 MHz, CDCl₃) δ −4.17; HRMS (ESI-TOF) m/z: [M+NH₄]⁺ Calcd for C₁₉H₃₉NO₁₁P 488.2255; Found 488.2174

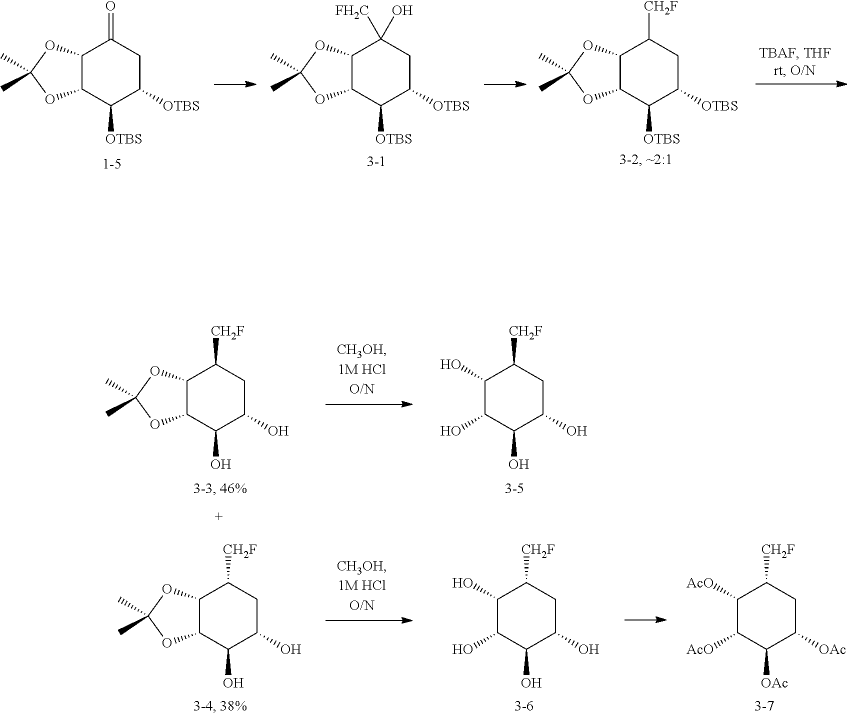

Synthesis of alcohol 3-1. To a stirred solution of ketone 1-5 (464 mg, 1.077 mmol) in THF/Et₂O (1:1) (17 mL) at −78° C. was added iodofluoromethane (0.086 M in pentane, 8.37 mL, 0.72 mmol) followed by MeLi·LiBr (1:1) (1.6 M in Et₂O, 1.34 mL, 2.15 mmol). The reaction mixture is stirred 5 min at −78° C. Next, the reaction mixture was treated with saturated aq NH₄Cl solution and extracted with Et₂O. The organic layer was dried over anhydrous MgSO₄, filtered, concentrated, and the resulting crude product was purified by column chromatography (5% Et₂O in hexane) to afford 3-1 (207 mg, 62%) as a colorless viscous liquid: [α]_{D}=+7.5 (c=0.96, CHCl₃); ¹H NMR (400 MHz, CDCl₃) δ4.23 (dd, J=8.9, 59.9 Hz, 1H), 4.13-4.11 (m, 2H), 4.10 (dd, J=8.9, 60.2 Hz, 1H), 4.08 (d, J=2.6 Hz, 1H), 3.90-3.85 (m, 1H), 2.04 (ddd, J=2.1, 4.1, 14.6 Hz, 1H), 1.78 (dd, J=4.9, 14.6 Hz, 1H), 1.55 (s, 3H), 1.37 (s, 1H), 0.91 (s, 9H), 0.89 (s, 9H), 0.12 (s, 9H), 0.10 (s, 3H); ¹³C NMR (101 MHz, CDCl₃): δ 109.4, 84.7 (d, J=174.9 Hz), 78.5, 73.5 (d, J=3.0 Hz), 72.4, 71.5, 70.8 (d, J=18.1 Hz), 33.3 (d, J=2.5 Hz), 26.3, 26.0, 25.9, 25.5, 18.1 (d, J=13.3 Hz), −4.3, −4.4, −4.5, −4.8; ¹⁹F NMR (377 MHz, CDCl₃): δ −228.3 (t, J=12.7, 47.4 Hz); IR (neat) v3468, 2930, 2370, 1255, 1082, 837 cm⁻¹; HRMS (ESI-TOF) m/z: [M+H]⁺ Calcd for C₂₂H₄₅FO₅Si₂ 465.2862; Found 465.2858.

Synthesis of fluoromethyl compound 3-2. To a stirred solution of alcohol 3-1 (40 mg, 0.086 mmol) in CH₂Cl₂ (1.2 mL) at −17° C. was added N,N-dimethylaminopyridine (63.2 mg, 0.516 mmol) followed by methyl 2-chloro-2-oxoacetate (47.5 μL, 0.516 mmol) in a dropwise manner. After 2 h, ice bath was removed, and the reaction mixture was warmed to room temperature and stirred 1 h. The reaction mixture was treated with cold saturated aq NaHCO₃ solution and extracted with EtOAc. The aqueous layer was separated, and the organic phase was dried over anhydrous MgSO₄, filtered, concentrated and the resulting crude product was quickly purified by column chromatography (33% Et₂O in hexane) to afford the corresponding ester: ¹H NMR (400 MHz, CDCl₃) δ 5.04 (dd, J=10.3, 48.1 Hz, 1H), 4.58 (dd, J=10.3, 45.7 Hz, 1H), 4.50 (d, J=6.7 Hz, 1H), 3.99 (t, J=6.5 Hz, 1H), 3.89 (s, 3H), 3.81 (dd, J=6.7, 7.9 Hz, 1H), 3.65 (m, 1H), 2.25 (m, 2H), 1.51 (s, 3 H), 1.32 (s, 3H), 0.90 (s, 9H), 0.89 (s, 9H), 0.13 (s, 3H), 0.10 (s, 3H), 0.09 (s, 3H), 0.08 (s, 3H). This crude material was dissolved in toluene (1 mL) in a microwavable glass tube. To this reaction mixture was added 2,2′-azobis(2-methylpropionitrile) (28.2 mg, 0.172 mmol) and the N₂ gas was bubbled through the solution for 30 min. In an another microwavable glass tube, neat n-tributyltin hydride (463 μL, 1.72 mmol) was added and the N₂ gas was bubbled through the solution for 30 min, after which time the reaction mixture was heated at 120° C. The former crude reaction mixture was added quickly into the latter and stirred at 120° C. for 2 h, after which time the solution was cooled to room temperature. The reaction mixture was treated with saturated aq NH₄Cl solution and extracted with EtOAc. The aqueous layer was separated and further extracted with EtOAc. The combined organic phases were washed with brine (20 mL), dried over anhydrous MgSO₄, filtered, concentrated and the resulting crude product was purified by column chromatography (5% Et₂O in hexane) to afford 3-2 (34 mg, 88% over the two steps) as a colorless viscous liquid which exhibits ˜2:1 diastereomeric ratio, as determined by ¹H NMR spectroscopy in CDCl₃ at 25° C.: ¹H NMR (400 MHz, CDCl₃) δ 4.67-4.26 (m, 2H), 4.24 (m, 0.6 H), 4.07-3.94 (m, 1.7 H), 3.81 (m, 0.4 H), 3.61-3.48 (m, 1.3 H), 2.38-2.13 (m, 1H), 1.81-1.68 (m, 1H), 1.64-1.43 (m, 1H), 1.50 (s, 2H), 1.47 (s, 1H), 1.36 (s, 1H), 1.35 (s, 2H), 0.93-0.90 (s, 18H), 0.15-0.07 (s, 12H); ¹³C NMR (101 MHz, CDCl₃): δ 109.2, 108.5, 85.4 (d, J=167.5 Hz), 84.6 (d, J=167.9 Hz), 81.7, 79.0, 78.4, 77.4, 73.3, 73.2, 71.8, 70.2, 35.9 (d, J=18.9 Hz), 35.2 (d, J=18.6 Hz), 29.6 (d, 4.9 Hz), 29.6 (d, J=3.1 Hz), 28.6, 28.0, 26.3, 26.2, 25.9, 25.8, 18.4, 18.3, 18.1, −3.5, −3.7, −4.0, −4.3, −4.6, −4.7, −4.8, −4.8; ¹⁹F NMR (377 MHz, CDCl₃): δ −222.7 (dt, J=12.7, 46.9 Hz), −231.1 (dt, J=29.9, 48.0 Hz); IR (neat) v 3698, 3621, 2929, 2369, 1470, 1245, 829 cm⁻¹; HRMS (ESI-TOF) m/z: [M+H]⁺ Calcd for C₂₂H₄₅FO₄Si₂ 449.2913; Found 449.2912.

Synthesis of diols 3-3 and 3-4. To a stirred solution of acetonide 3-2 (129 mg, 0.288 mmol) in THF (3 mL) was added tetra-n-butylammonium fluoride (1.0 M in THF, 1.15 mL, 1.15 mmol) and stirred for 12 h. The reaction mixture was then treated with saturated aq NH₄Cl solution and extracted with ethyl acetate. The aqueous layer was separated and further extracted with EtOAc. The combined organic phases were washed with brine, dried over anhydrous MgSO₄, filtered, concentrated and the resulting crude product was purified by column chromatography (20% Acetone in DCM) to afford isomer 3-4 (21.8 mg, 30%) as a white solid and isomer 3-3 (37.0 mg, 51%) as a white solid. Data for 3-3: [α]_{D}=+30.7 (c=1.4, acetone); ¹H NMR (400 MHz, CDCl₃) δ 4.59 (ddd, J=7.6, 8.9, 46.7 Hz, 1H), 4.42 (ddd, J=6.9, 8.9, 47.1 Hz, 1H), 4.26 (t, J=4.3 Hz, 1H), 3.93 (m, 1 H), 3.57-3.44 (m, 2H), 2.50 (d, J=2.7 Hz, 1H), 2.34 (d, J=2.4 Hz, 1H), 2.29 (m, 1 H), 1.88 (m, 1H), 1.51 (s, 3H), 1.48 (m, 1H), 1.36 (s, 3H); ¹³C NMR (101 MHz, CDCl₃) δ 109.9, 85.0, 83.4, 80.7, 78.7, 73.7 (d, J=6.1 Hz), 71.0 (d, J=1.5 Hz), 36.3 (d, J=19.1 Hz), 28.6 (d, J=5.7 Hz), 28.5, 26.5; ¹⁹F NMR (377 MHz, CDCl₃): δ −222.6 (dt, J=12.8, 46.7 Hz); IR (neat) v 3721, 3694, 3624, 3597, 3214, 2369, 1366, 1218 1024 cm⁻¹; HRMS (ESI-TOF) m/z: [M+H]⁺ Calcd for C₁₀H₁₇FO₄ 221.1184; Found 221.1183. Data for 3-4: [α]_{D}=+17.4 (c=1.1, acetone); ¹H NMR (500 MHz, CDCl₃) δ 4.56 (ddd, J=4.5, 9.3, 36.7 Hz, 1H), 4.46 (ddd, J=4.0, 9.3, 36.0 Hz, 1H), 4.20 (t, J=5.7 Hz, 1H), 4.00 (t, J=6.6 Hz, 1H), 3.77-3.55 (m, 2H), 2.52 (s, 1H), 2.47 (s, 1H), 2.34 (m, 1H), 1.87 (m, 2H), 1.52 (s, 3H), 1.37 (s, 3H); ¹³C NMR (101 MHz, CDCl₃): δ 109.3, 85.6 (d, J=170.0 Hz), 79.0 (d, J=1.1 Hz), 76.5, 74.8 (d, J=4.8 Hz), 68.8 (d, J=1.9 Hz), 36.3 (d, J=18.8 Hz), 29.8 (d, J=3.1 Hz), 28.2, 25.9; ¹⁹F NMR (377 MHz, CDCl₃): δ −225.0 (dt, J=31.3, 47.5 Hz); IR (neat) v3712, 3695, 3677, 3665, 2919, 2852, 1446, 1369, 1004 cm⁻¹; HRMS (ESI-TOF) m/z: [M+H]⁺ Calcd for C₁₀H₁₇FO₄ 221.1184; Found 221.1182.

Synthesis of tetraol 3-5. To a stirred solution of diol 3-3 (24 mg, 93.7 μmol) in CH₃OH (4.8 mL) was added 1 M HCl (1.2 mL) and stirred for 2 h. The reaction mixture was concentrated under reduced pressure and the resulting crude product was purified by column chromatography (20% CH₃OH in EtOAc) to afford 3-5 (15.4 mg, 91%) as a colorless viscous liquid: [α]_{D}=+2.8 (c=1.4, CH₃OH); ¹H NMR (400 MHz, CD₃OD) δ4.50 (dt, J=8.5, 47.5 Hz, 1H), 4.32 (ddd, J=6.5, 8.7, 47.3 Hz, 1H), 3.94 (m, 1H), 3.53 (t, J=9.3 Hz, 1H), 3.50-3.39 (m, 1H), 3.29 (dd, J=3.0, 9.5 Hz, 1H), 1.92 (m, 1H), 1.64 (dt, J=4.2, 12.5 Hz, 1H), 1.47 (q, J=12.4 Hz, 1H), 1.02 (d, J=6.7 Hz, 1H); ¹³C NMR (151 MHz, CD₃OD) δ 83.7 (d, J=166.7 Hz), 75.2, 74.7, 72.0, 68.5 (d, J=5.3 Hz), 37.9 (d, J=18.6 Hz), 28.0 (d, J=6.5 Hz); ¹⁹F NMR (377 MHz, CD₃OD) δ −225.0 (dt, J=12.4, 47.4 Hz); IR (neat) v3728, 3694, 2345, 1598, 1044 cm⁻¹; HRMS (ESI-TOF) m/z: [M+NH₄]⁺ Calcd for C₇H₁₇NFO₄ 198.1136; Found 198.1139.

Synthesis of tetraol 3-6. To a stirred solution of diol 3-4 (18 mg, 70.3 μmol) in CH₃OH (4.8 mL) was added 1 M HCl (1.2 mL) and stirred for 2 h. The reaction mixture was concentrated under reduced pressure and the resulting crude product was purified by column chromatography (20% CH₃OH in EtOAc) to afford 3-6 (12.2 mg, 96%) as a colorless viscous liquid: [α]_{D}=+5.8 (c=0.3, CH₃OH); ¹H NMR (500 MHz, CD₃OD) δ 4.59 (ddd, J=5.2, 9.2, 47.8 Hz, 1H), 4.47 (ddd, J=4.4, 9.2, 47.7 Hz, 1H), 3.85-3.78 (m, 2H), 3.75-3.68 (m, 2H), 2.21 (m, 1H), 1.87-1.83 (m, 2 H); ¹³C NMR (151 MHz, CD₃OD) δ 85.9 (d, J=167 Hz), 74.8, 73.9, 71.4, 69.7 (d, J=3.8 Hz), 37.8 (d, J=17.9 Hz), 30.5 (d, J=3.5 Hz); ¹⁹F NMR (377 MHz, CD₃OD) 5-229.0 (broad signal); IR (neat) v 3731, 3691, 2346, 1645, 1064 cm⁻¹; HRMS (ESI-TOF) m/z: [M+NH₄]⁺ Calcd for C₇H₁₇NFO₄ 198.1136; Found 198.1141.

Synthesis of acetate 3-7. To a stirred solution of tetrol 3-6 (3 mg, 16.7 μmol) in pyridine (0.6 mL) was added Ac₂O (0.02 mL, 0.2 mmol) and stirred for 12 h at 50° C. The reaction mixture was concentrated under reduced pressure and co-evaporated with toluene and the resulting crude product was purified by column chromatography (30% EtOAc in hexanes) to afford 3-7 (5.2 mg, 89%) as a colorless viscous liquid: [α]_{D}=+12.6 (c=0.6, CHCl₃); ¹H NMR (400 MHz, CDCl₃) δ 5.32-5.25 (m, 2H), 5.18 (d, J=3.3, 7.5 Hz, 1H), 5.08-5.02 (m, 1H), 4.56 (ddd, J=4.4, 9.7, 47.5 Hz, 1H), 4.49 (ddd, J=4.4, 9.7, 47.1 Hz, 1H), 2.37 (m, 1H), 2.09 (s, 3H), 2.06 (s, 3H), 2.05 (s, 3H), 2.04 (s, 3H), 2.05-2.01 (m, 2H); ¹³C NMR (151 MHz, CDCl₃) δ 170.0, 170.0, 169.9, 169.7, 84.0 (d, J=171.2 Hz), 70.2, 69.6 (d, J=3.1 Hz), 69.5 (d, J=2.5 Hz), 69.4 (d, J=1.8 Hz), 35.9 (d, J=19.5 Hz), 27.6 (d, J=3.1 Hz, 21.1, 21.0, 20.9, 20.8; ¹⁹F NMR (377 MHz, CDCl₃) δ −223.9 (broad signal); IR (cast film) v 2919, 2849, 1748, 1369, 1218, 1040 cm⁻¹; HRMS (ESI-TOF) m/z: [M+NH₄]⁺ Calcd for C₁₅H₂₅NO₈ 366.1559; Found 366.1557.

Aldehyde 1-29 was prepared from L-quebrachitol according to literature reported procedure

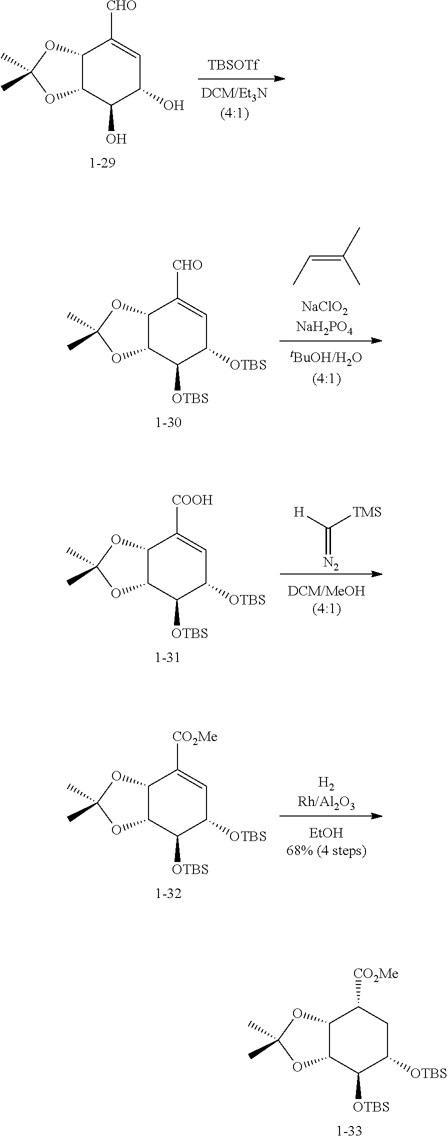

Synthesis of methyl ester 1-33. To a solution of aldehyde 1-29 ((Monda, S.; Sureshan, K. M. J. Org. Chem. 2016, 81, 11635, 0.161 mmol, 34.5 mg, 1.0 equiv) in CH₂Cl₂:Et₃N (4:1, c=0.1 M) at 0° C. was added tert-butyldimethylsilyl trifluoromethanesulfonate (4.0 equiv). After stirring at room temperature for 1 h, the reaction mixture was quenched with saturated aqueous NaHCO₃. The aqueous layer was extracted with Et₂O. The combined organic layers were washed with brine, dried over Na₂SO₄, and evaporated in vacuo. The residue (1.0 equiv) was dissolved in ^{t}BuOH:H₂O (4:1, c=0.01 M). 2-Methyl-2-butene (100 equiv), NaH₂PO₄ (11 equiv) and NaClO₂ (10 equiv) were added at 0° C. After stirring at 0° C. for 10 min, the reaction mixture was stirred at room temperature for 1 h. The reaction mixture was quenched with H₂O. The aqueous layer was extracted with ethyl acetate. The combined organic layers were washed with brine, dried over Na₂SO₄, and evaporated in vacuo. The residue (1.0 equiv) was redissolved in CH₂Cl₂:MeOH (4:1, c=0.01 M) and (trimethylsilyl)diazomethane (5.0 equiv) was added. The reaction mixture was stirred at room temperature for 1 h. The solvent was removed under reduced pressure, and the residue (1.0 equiv) was dissolved in EtOH (c=0.1 M) and Rh/A12O₃ (0.5 equiv) was added. The reaction mixture was stirred at rt under H₂ (50 atm). After 12 h, the reaction mixture was filtered through Celite and the filtrate was evaporated in vacuo. The residue was purified by column chromatography (ⁿhexane:Et₂O 10:1) on silica gel to give 1-33 (52 mg) as a white solid (isolated yield 68%). m.p.: 105-106° C.; ¹H NMR (600 MHz, CDCl₃): δ 4.53 (dd, J=4.5, 4.1 Hz, 1H), 4.01 (t, J=6.8 Hz, 1H), 3.73 (s, 3H), 3.59 (dd, J=6.9, 6.5 Hz, 1H), 3.52-3.48 (m, 1H), 2.74 (dt, J=13.4, 3.5 Hz, 1H), 1.98 (dt, J=13.4, 3.8 Hz, 1H), 1.91-1.85 (m, 1H), 1.48 (s, 3H), 1.32 (s, 3H), 0.89 (d, J=3.0 Hz, 18H), 0.11-0.07 (m, 12H); ¹³C NMR (151 MHz, CDCl₃): δ 172.1, 109.4, 80.9, 77.7, 74.1, 72.9, 52.2, 40.4, 28.8, 27.6, 26.20, 26.18, 25.9, 18.3, 18.2, −3.7, −3.8, −4.2, −4.3; IR (neat): v 3182, 2950, 2931, 2857, 1747, 1306, 1255, 1108, 1046, 827, 780 cm⁻¹; HRMS (ESI): m/z [M+H]⁺ calcd. for C₂₃H₄₇O₆Si₂: 475.2911; found: 475.2910; [α]_{D}²⁰=29.3 (c=0.1, MeOH).

Synthesis of Tetrol 1-34.

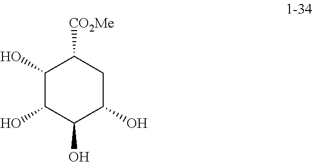 [SMILES: COC(=O)[C@@H]1C[C@H](O)[C@@H](O)[C@H](O)[C@@H]1O]

1-33 (0.025 mmol, 12.0 mg) was dissolved in methanol:1N HCl (4:1, c=0.05 M). After stirring at room temperature for 5 h, the solvents were removed in vacuo. The residue was purified by column chromatography (CH₂Cl₂:MeOH 4:1) on silica gel to give 1-34 (5.2 mg) as white solid (isolated yield 76%). ¹H NMR (400 MHz, MeOD): δ 4.26 (t, J=2.7 Hz, 1H), 3.70 (s, 3H), 3.52-3.47 (m, 1H), 3.41-3.37 (m, 1H), 3.33-3.32 (m, 1H), 2.61-2.56 (m, 1H), 1.94-1.89 (m, 2H); ¹³C NMR (151 MHz, MeOD): δ 174.5, 76.1, 75.7, 72.9, 71.8, 52.3, 44.3, 29.6; IR (neat): v 3178, 2921, 2853, 1718, 1578, 1421, 1298, 847 cm⁻¹; HRMS (ESI): m/z [M+H]⁺ calcd. for C₈H₁₅O₆: 207.0863; found: 207.0861; [α]_{D}²⁰=−14.0 (c=0.1, MeOH).

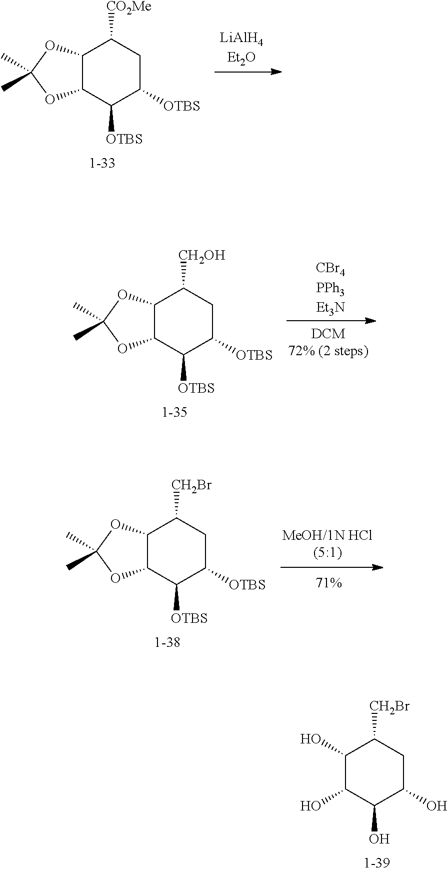

Synthesis of alkylbromide 1-38. LiAlH₄ (3.0 equiv) was added in portions to a solution of 1-33 (0.058 mmol, 27.4 mg, 1.0 equiv) in Et₂O (c=0.03 M). After heating at reflux for 16 h, the reaction mixture was quenched with wet Et₂O. The aqueous layer was extracted with ethyl acetate. The combined organic layers were washed with brine, dried over Na₂SO₄, and evaporated. The residue (1.0 equiv) was dissolved in CH₂Cl₂ (c=0.05 M). PPh₃ (1.0 equiv), CBr₄ (1.0 equiv) and Et₃N (1.0 equiv) were added. After stirring at room temperature for 48 h, the solvents were removed in vacuo. The residue was purified by column chromatography (ⁿhexane:Et₂O 40:1) on silica gel to give 1-38 (21.2 mg) as foamy solid (isolated yield 72%). Data for 1-38: ¹H NMR (600 MHz, CDCl₃): δ 4.29 (dd, J=4.3, 4.1 Hz, 1H), 3.92 (t, J=5.8 Hz, 1H), 3.54-3.50 (m, 2H), 3.47-3.43 (m, 1H), 3.34 (dd, J=9.8, 6.9 Hz, 1H), 2.12-2.06 (m, 1H), 1.83 (dt, J=12.8, 3.6 Hz, 1H), 1.51-1.47 (m, 1H), 1.47 (s, 3H), 1.33 (s, 3H), 0.90 (s, 18H), 0.12-0.08 (m, 12H); ¹³C NMR (151 MHz, CDCl₃): δ 109.0, 81.6, 78.9, 74.5, 73.2, 38.0, 34.5, 32.8, 29.9, 28.0, 26.3, 26.2, 18.34, 18.32, −3.6, −3.7, −4.1, −4.3; IR (neat): v 2954, 2932, 2861, 1474, 1384, 1257, 1097, 1052, 1034, 803 cm⁻¹; HRMS (ESI): m/z [M+H]⁺ calcd. for C₂₂H₄₆BrO₄Si₂: 509.2113; found: 509.2113; [α]_{D}²⁰=−6.3 (c=0.3, MeOH).

Synthesis of tetraol 1-39. 1-38 (0.029 mmol, 14.9 mg) was dissolved in methanol:1 N HCl (4:1, c=0.05 M). After stirring at room temperature for 3 h, the solvents were removed in vacuo. The residue was purified by column chromatography (CH₂Cl₂:MeOH 15:1, then 2:1) on silica gel to give 1-39 (5.0 mg) as colorless oil (isolated yield 71%). ¹H NMR (600 MHz, MeOD): δ4.02 (brs, 1H), 3.55-3.49 (m, 2H), 3.42-3.35 (m, 2H), 3.27 (dd, J=9.6, 2.9 Hz, 1H), 1.87-1.79 (m, 2H), 1.53-1.47 (m, 1H); ¹³C NMR (151 MHz, MeOD): δ 76.4, 76.1, 73.2, 71.3, 41.6, 35.2, 32.9; IR (neat): v 3372, 2924, 2857, 1451, 1257, 1116, 1072, 1049, 989 cm⁻¹; HRMS (ESI): m/z [M+Na]⁺ calcd. for C₇H₁₃BrNaO₄: 262.9889; found: 262.9886; [α]_{D}²⁰=−9.2 (c=0.12, MeOH).

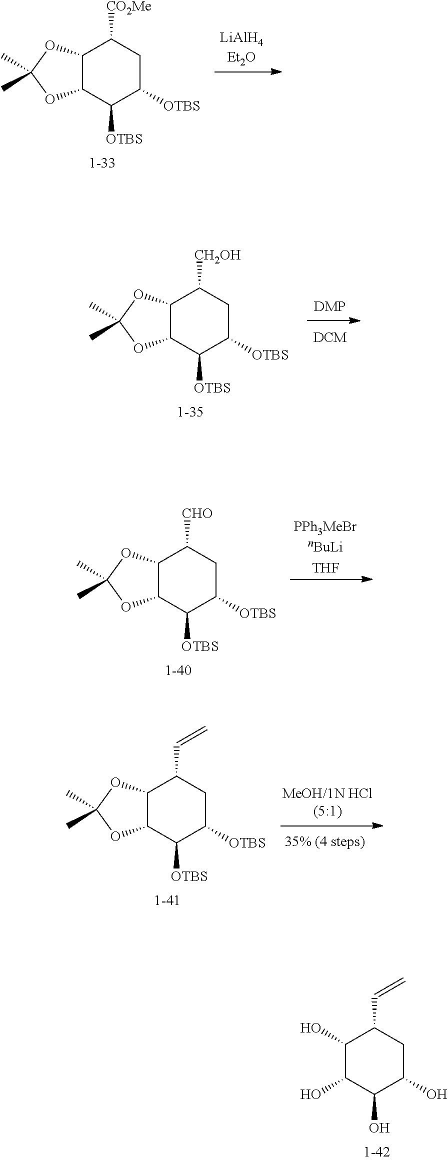

Synthesis of alkene 1-42. LiAlH₄ (3.0 equiv) was added in portions to a solution of 1-33 (0.066 mmol, 31.1 mg, 1.0 equiv) in Et₂O (c=0.03 M). After heating at reflux for 16 h, the reaction mixture was quenched with wet Et₂O. The aqueous layer was extracted with ethyl acetate. The combined organic layers were washed with brine, dried over Na₂SO₄, and evaporated in vacuo. The residue (1.0 equiv) was dissolved in CH₂Cl₂ (c=0.1 M), and Dess-Martin periodinane (1.2 equiv) was added. After stirring at room temperature for 30 min, the solvents were removed in vacuo to obtain the crude aldehyde 1-40. To a solution of PPh₃MeBr (3.0 equiv) in THF under N₂ atmosphere was added ⁿBuLi (2.5 M in hexane, 3.0 equiv) at −78° C. The mixture was allowed to warm to room temperature for 30 min, and then was cooled to −78° C. before the crude aldehyde 1-40 (1.0 equiv) was added. The reaction mixture was warmed up to room temperature for 3 h. The reaction mixture was quenched with water and extracted with Et₂O. The organic layer was dried over Na₂SO₄, and concentrated to obtain the crude alkene 1-41. The residue (0.055 mmol) was dissolved in methanol:1 N HCl (4:1, c=0.05 M). After stirring at room temperature for 3 h, the solvents were removed in vacuo. The residue was purified by column chromatography (CH₂Cl₂:MeOH 15:1, then 2:1) on silica gel to give 1-42 (4.0 mg) as colorless oil (isolated yield 35%). ¹H NMR (600 MHz, MeOD): δ 5.98-5.90 (m, 1H), 5.08 (dt, J=17.3, 1.6 Hz, 1H), 5.04 (dt, J=10.4, 1.5 Hz, 1H), 3.81 (brs, 1H), 3.53-3.49 (m, 1H), 3.45-3.39 (m, 1H), 3.33-3.31 (m, 1H), 2.24-2.19 (m, 1H), 1.78-1.63 (m, 2H); ¹³C NMR (151 MHz, MeOD): δ 140.8, 115.1, 76.5, 76.2, 74.1, 73.7, 42.8, 33.2; IR (neat): v3318, 2932, 2876, 1645, 1421, 1257, 1123, 1071, 1049, 989, 903 cm⁻¹; HRMS (ESI): m/z [M+Na]⁺ calcd. for C₈H₁₄NaO₄: 197.0784; found: 197.0787; [α]_{D}²⁰=−5.4 (c=0.3, MeOH).

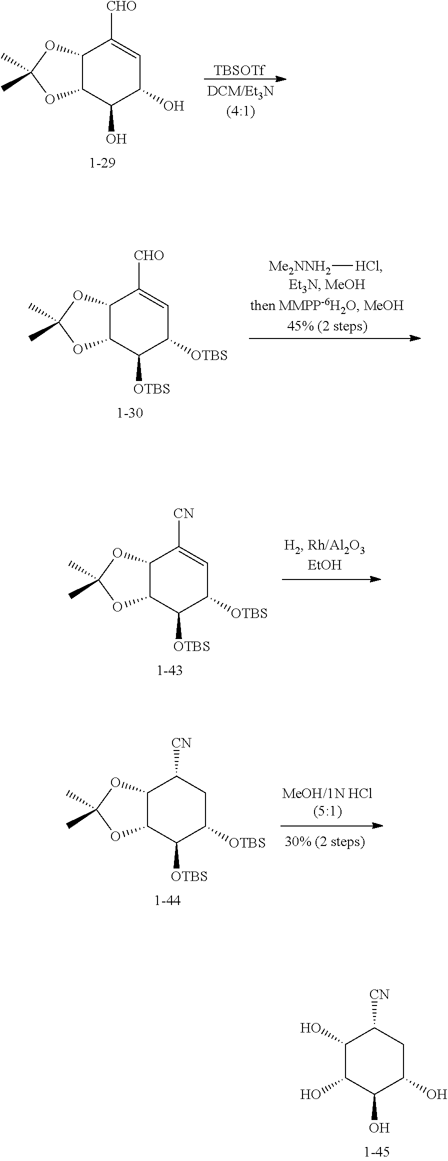

Synthesis of nitrile 1-43. To a solution of aldehyde 1-29 (0.115 mmol, 24.6 mg, 1.0 equiv) in CH₂Cl₂:Et₃N (4:1, c=0.1 M) at 0° C. was added tert-butyldimethylsilyl trifluoromethanesulfonate (1.0 equiv). After stirring at room temperature for 1 h, the reaction mixture was quenched with saturated aqueous NaHCO₃. The aqueous layer was extracted with Et₂O. The combined organic layers were washed with brine, dried over Na₂SO₄, and evaporated in vacuo to give crude 1-30. Dimethylhydrazine hydrochloride (1.5 equiv) and Et₃N (1.5 equiv) in MeOH (c=0.175 M) were stirred at room temperature for 10 min. Crude 1-30 (1.0 equiv) was added to the reaction mixture. The reaction mixture was stirred at room temperature for 2 h. The above reaction solution was then added dropwise to magnesium monoperoxyphthalate hexahydrate (2.5 equiv) in MeOH (c=0.7 M) at 0° C. After stirring at 0° C. for 10 min, the reaction mixture was quenched with H₂O. The aqueous layer was extracted with Et₂O. The combined organic layers were washed with brine, dried over Na₂SO₄, and evaporated in vacuo. The residue was purified by column chromatography (ⁿhexane:Et₂O 12:1) on silica gel to give 1-43 (22.8 mg) as foamy solid (isolated yield 45%)¹H NMR (600 MHz, CDCl₃): δ 6.46 (dd, J=3.2, 0.8 Hz, 1H), 4.62 (d, J=6.4 Hz, 1H), 4.18 (dd, J=6.0, 6.0 Hz, 1H), 4.09-4.07 (m, 1H), 3.80 (dd, J=5.8, 5.7 Hz, 1H), 1.47 (s, 3H), 1.37 (s, 3H), 0.90 (s, 9H), 0.88 (s, 9H), 0.12 (s, 3H), 0.11 (s, 3H), 0.095 (s, 3H), 0.088 (s, 3H); ¹³C NMR (151 MHz, CDCl₃): δ 146.5, 117.0, 113.1, 111.6, 77.2, 72.5, 71.4, 69.7, 29.8, 27.7, 26.00, 25.99, 25.9, 25.8, 18.2, 18.1, −4.1, −4.3, −4.4; IR (neat): v 2929, 2859, 1679, 1463, 1378, 1258, 1096, 837, 779 cm⁻¹; HRMS (ESI): m/z [M+H]⁺ calcd. For C₂₂H₄₂NO₄Si₂: 440.2652; found: 440.2650; [α]_{D}²⁰=50.1 (c=0.6, CH₂Cl₂).

Synthesis of tetraol 1-45. To 1-43 (0.048 mmol, 21.2 mg, 1.0 equiv) in EtOH (c=0.05 M) was added Rh/A12O₃ (0.5 equiv). The reaction mixture was stirred at rt under H₂ (50 atm). After 12 h, the reaction mixture was filtered through Celite and the filtrate was evaporated in vacuo. The residue was dissolved in methanol:1 N HCl (4:1, c=0.05 M). After stirring at room temperature for 3 h, the solvents were removed in vacuo. The residue was purified by column chromatography (CH₂Cl₂:MeOH 15:1) on silica gel to give 1-45 (2.5 mg) as colorless oil (isolated yield 30%)¹H NMR (600 MHz, MeOD): δ 4.05 (dd, J=2.4, 2.4 Hz, 1H), 3.51-3.48 (m, 1H), 3.38-3.35 (m, 1H), 3.26 (dd, J=9.6, 2.8 Hz, 1H), 2.94-2.91 (m, 1H), 2.00-1.97 (m, 2H); ¹³C NMR (151 MHz, MeOD): δ 121.4, 75.6, 74.9, 72.2, 70.7, 31.4, 31.3; IR (neat): v2928, 2859, 1679, 1463, 1378, 1258, 1196, 1100, 837, 776, 683 cm⁻¹; HRMS (ESI): m/z [M+Na]⁺ calcd. for C₇H₁₁NNaO₄: 196.0586; found: 196.0590; [α]_{D}²⁰=2.9 (c=0.1, MeOH).

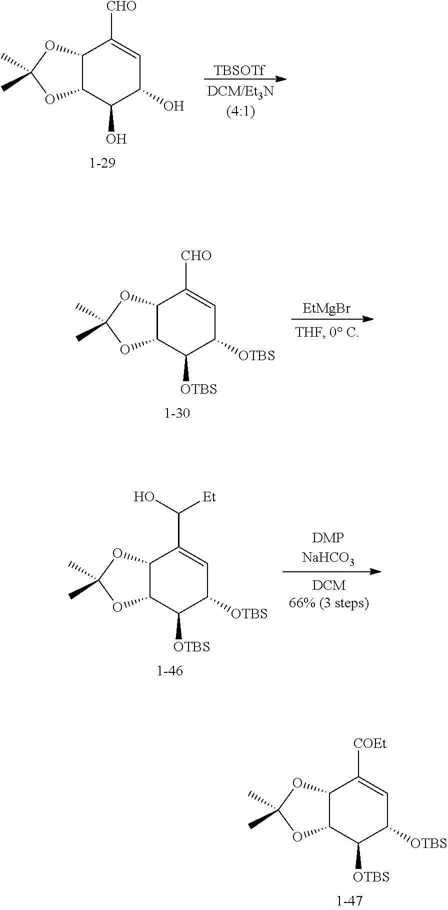

Synthesis of ethyl ketone 1-47. To a solution of aldehyde 1-29 (0.1 mmol, 21.4 mg, 1.0 equiv) in CH₂Cl₂:Et₃N (4:1, c=0.1 M) at 0° C. was added tert-butyldimethylsilyl trifluoromethanesulfonate (1.0 equiv). After stirring at room temperature for 1 h, the reaction mixture was quenched with saturated aqueous NaHCO₃. The aqueous layer was extracted with Et₂O. The combined organic layers were washed with brine, dried over Na₂SO₄, and evaporated in vacuo. The residue (1.0 equiv) was dissolved in THF (c=0.1 M), and EtMgBr (3.0 equiv) was added at 0° C. After stirring at 0° C. for 2 h, the reaction mixture was quenched with saturated aqueous NH₄Cl. The aqueous layer was extracted with Et₂O. The combined organic layers were washed with brine, dried over Na₂SO₄, and evaporated in vacuo. The residue (1.0 equiv) was dissolved in CH₂Cl₂ (c=0.1 M). Dess-Martin periodinane (1.2 equiv) and NaHCO₃ (3.0 equiv) were added. After stirring at room temperature for 30 min, the solvents were removed in vacuo. The residue was purified by column chromatography (ⁿhexane:Et₂O 15:1) on silica gel to give 1-47 (31.8 mg) as foamy solid (isolated yield 66%)¹H NMR (400 MHz, CDCl₃): δ 6.64 (d, J=2.3 Hz, 1H), 5.01 (d, J=6.6 Hz, 1H), 4.16 (ddd, J=7.2, 2.2, 1.2 Hz, 1H), 4.07 (dd, J=7.6, 6.6 Hz, 1H), 3.69 (t, J=7.5 Hz, 1H), 2.79 (dq, J=17.7, 7.2 Hz, 1H), 2.63 (dq, J=17.7, 7.2 Hz, 1H), 1.45 (s, 3H), 1.40 (s, 3H), 1.12 (t, J=7.2 Hz, 3H), 0.94 (s, 9H), 0.90 (s, 9H), 0.14-0.09 (m, 12H); ¹³C NMR (101 MHz, CDCl₃): δ 200.3, 141.5, 134.9, 110.3, 78.5, 74.7, 71.7, 70.8, 31.9, 29.8, 28.2, 26.3, 26.2, 26.1, 18.4, 18.3, 8.0, −3.7, −3.8, −3.9, −4.3; IR (neat): v 2930, 2855, 1715, 1682, 1462, 1381, 1250, 1202, 1145, 1115, 1073, 838, 779 cm⁻¹; HRMS (ESI): m/z [M+H]⁺ calcd. for C₂₄H₄₇O₅Si₂: 471.2962; found: 471.2961; [α]_{D}²⁰=15.2 (c=1.0, CH₂Cl₂).

Synthesis of ketone 1-49

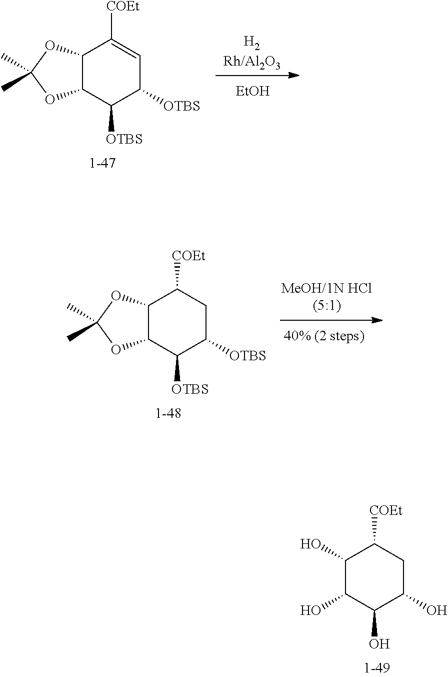

Synthesis of ketone 1-49. To 1-47 (0.05 mmol, 23.6 mg, 1.0 equiv) in EtOH (c=0.05 M) was added Rh/Al₂O₃ (0.5 equiv). The reaction mixture was stirred at rt under H₂ (50 atm). After 12 h, the reaction mixture was filtered through Celite and the filtrate was evaporated in vacuo. The residue was dissolved in methanol:1 N HCl (4:1, c=0.05 M). After stirring at room temperature for 3 h, the solvents were removed in vacuo. The residue was purified by column chromatography (CH₂Cl₂:MeOH 15:1, then 2:1) on silica gel to give 1-49 (4.1 mg) as colorless oil (isolated yield 40%)¹H NMR (600 MHz, MeOD): δ 4.32 (dd, J=2.7, 2.4 Hz, 1H), 3.50-3.47 (m, 1H), 3.41-3.38 (m, 1H), 3.36 (dd, J=9.5, 2.9 Hz, 1H), 2.68-2.51 (m, 3H), 1.87-1.84 (m, 2H), 1.02 (t, J=7.2 Hz, 3H); ¹³C NMR (151 MHz, MeOD): δ212.8, 76.2, 76.0, 73.2, 71.7, 50.9, 34.2, 29.1, 7.9; IR (neat): v2939, 2852, 1713, 1672, 1256, 1202, 1070, 841, 789 cm⁻¹; HRMS (ESI): m/z [M+H]⁺ calcd. for C₉H₁₇O₅: 205.1076; found: 205.1072; [α]_{D}²⁰=−24.1 (c=0.1, MeOH).

Cell-based Assay

CHO K1 cells were grown in a T175 flask, in F12 medium supplemented with 10% PBS, at 37° C., 5% CO₂. 350 cells were seeded in every well of a 384-well plate (Corning 4680, 45 μL final) and incubated at 37° C. overnight. Inhibitors were stored as 20 mM stock solution in DMSO. Inhibitors were diluted to the desired concentration in CHO cells culture medium and were dispensed into the wells of the plate (50 μL final well volume). An equivalent amount of DMSO (vehicle) was dispensed into control wells. Cells were incubated in the presence of inhibitors for five days. Following treatment, the culture medium was removed and cells were washed four times with 60 μL of PBS-T using a combination washer/dispenser (EL406, Biotek). 50 μL of 4% PFA in PBS-T was then dispensed into the plate and cells were incubated for 15 min at room temperature before being washed four times with 60 μL of PBS-T. Cells were subsequently incubated for 45 minutes at room temperature with 5% Bovine Serum Albumin (BSA) in PBS-T to prevent non-specific lectin binding. The BSA solution was then replaced by 50 μL of a solution of 5 μg/mL of Fluorescein-conjugated Aleuria Aurentia Lectin (AAL) (Vector Laboratories) and cells were kept at room temperature in the dark for 1 h. Negative controls were co-incubated with AAL and 100 mM fucose as a lectin antagonist. Cells were subsequently washed four times with 60 μL PBS-T. Then, 50 μL of 1 μg/mL Hoechst solution in PBS was dispensed into each well and cells were directly imaged using an ImageXpress MicroXLS high content microscope (Molecular Devices). Four sites per wells were imaged using DAPI and FITC channels (50 ms and 300 ms exposure respectively). Images were processed using MetaXpress software. Briefly, for each site, the integrated average fluorescence was quantified and normalized for the corresponding number of cells determined with nuclear staining. Results from each site were combined to provide a single value for each well. At least four independent wells were imaged for each condition providing four biological replicates. IC₅₀ curves were plotted using GraphPad Prism. Results were normalized on the average value for vehicle wells (DMSO treated).

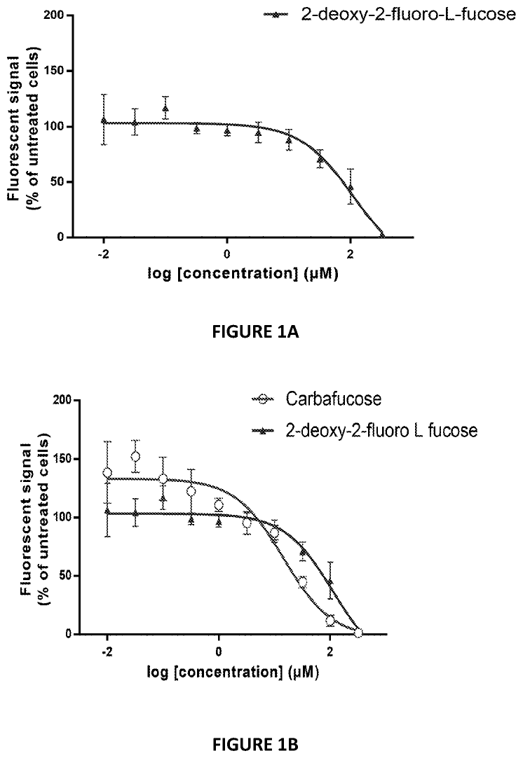

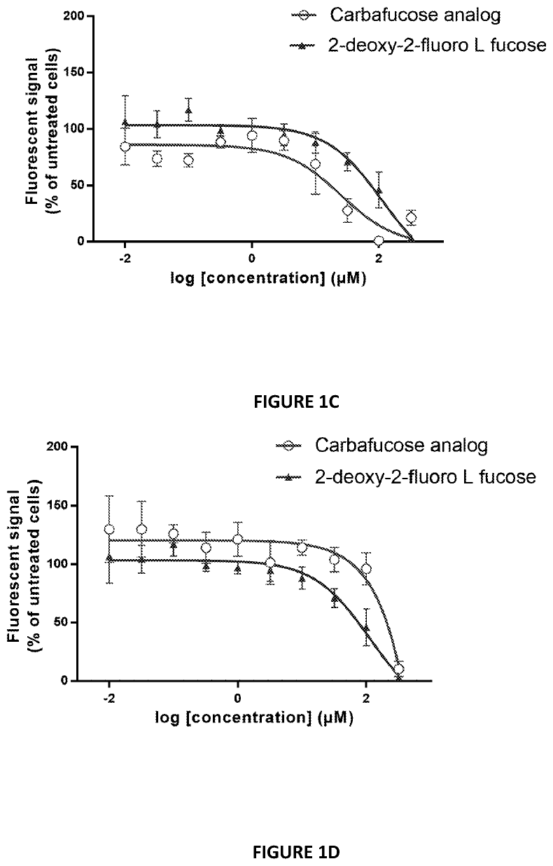

We assessed the potency of carbafucose and selected analogs through dose-response experiments over a 3.2 nM-100 μM range of concentrations in the cell-based assay. The cell-based fucosylation antagonist 2-deoxy-2-fluoro-L-fucose (2FFuc) was used as a control (FIGS. 1A-D).

Under the assay conditions, an IC₅₀ of 137±79 μM was measured with 2FFuc; the most potent compound was carbafucose with an IC₅₀ of IC₅₀ of 16.1±7.7 μM, as follows.

| Compound | IC₅₀ |
|---|---|
| 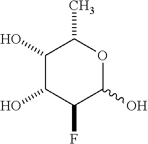 | 137 ± 79 μM |
| 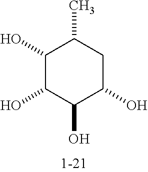 | 16.1 ± 7.7 μM |
| 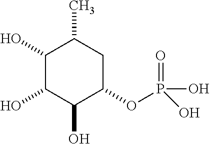 | 39.5 ± 30.3 μM |
| 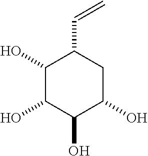 | 137 ± 79 μM |

The following compounds did not exhibit satisfactory results under the assay conditions.

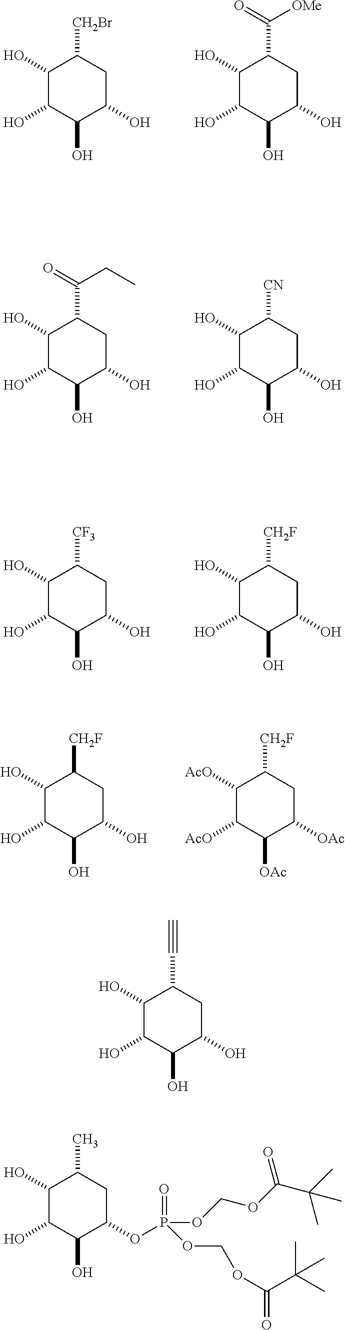

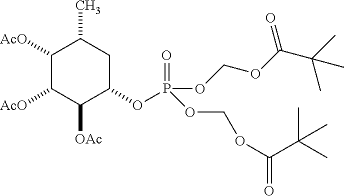

All citations are hereby incorporated by reference.

### Other Embodiments

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. It is to be understood that specific embodiments may be combined in any manner and in any number to create additional embodiments and any permutations and combinations of the embodiments should be considered disclosed by the description of the present application unless the context indicates otherwise. Numeric ranges are inclusive of the numbers defining the range. Recitation of numeric ranges of values herein is merely intended to serve as a shorthand method of referring individually to each separate value falling within the range. Unless otherwise indicated herein, each individual value is incorporated into the specification as if it were individually recited herein. By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc. The terms “a” and “an” and “the”” and similar reference used in the context of describing the invention are to be construed to cover both the singular and the plural, unless otherwise indicated herein or clearly contradicted by context. In the description, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples.

1. A method of inhibiting fucosylation of a protein, or fragment or derivative thereof, comprising contacting a eukaryotic cell or a mammal with a compound of Formula (I) or a salt thereof: 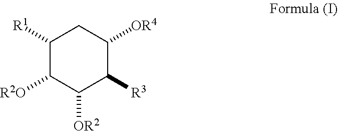
wherein
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), wherein R⁵ and R⁶ are each independently H, (C₁-C₆)alkyl, (CH₂)₂SC(═O)CH₃, CH₂OC(═O)OR⁷ or CH₂OC(═O)R⁷ wherein R7 is C1-C8 alkyl, or wherein R⁵ and R⁶ are connected to form a ring,
wherein fucosylation of the protein is reduced by at least 5% in the eukaryotic cell or mammal relative to the amount of fucosylation of the protein in the absence of administration of the compound.

2. The method of claim 1 wherein the protein comprises an N-glycan.

3. The method of claim 2 wherein the compound is not incorporated into the N-glycan.

4. The method of claim 1 wherein the protein is an antibody.

5. The method of claim 1 wherein R¹ is CH₃ or CHCH₂, R² is H, R³ is OH, and R⁴ is H or —P(═O)(OR⁵)(OR⁶), wherein R⁵ and R⁶ are H.

6. The method of claim 1 wherein the compound is

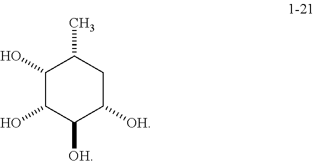

7. The method of claim 3 wherein the mammal has a cancer, an autoimmune disease, an inflammatory disease, or an infectious disease.

8. The method of claim 7 further comprising administering a cancer-associated antigen or an antigenic fragment thereof as an immunogen to the mammal having a cancer.

9. The method of claim 3 wherein the mammal is a human.

10. The method of claim 3 wherein the salt is a pharmaceutically acceptable salt.

11. A mammalian cell culture medium comprising an effective amount of a compound of Formula (I) or a salt thereof: 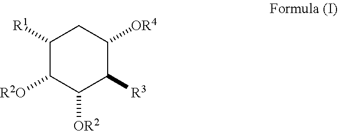
wherein
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), where R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or where R⁵ and R⁶ are connected to form a ring.

12. The mammalian culture medium of claim 11 wherein the medium is useful for the production of a fucose-deficient protein, or fragment or derivative thereof.

13. The mammalian culture medium of claim 12 wherein the effective amount is an amount of the compound is an amount sufficient to decrease fucose incorporation into a sugar chain of the fucose-deficient protein or fragment or derivative thereof by at least 50%.

14. The mammalian culture medium of claim 11 wherein the mammalian cell culture medium is a Chinese hamster ovary cell culture medium.

15. A method of treating a cancer, an autoimmune disease, an infectious disease, an inflammatory disease, or sickle cell disease comprising administering an effective amount of a compound of Formula (I) or a pharmaceutically acceptable salt thereof: 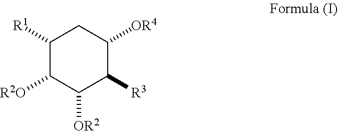
wherein
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶), wherein R⁵ and R⁶ are each independently H, (C₁-C₆)alkyl, (CH₂)₂SC(═O)CH₃, CH₂OC(═O)OR⁷ or CH₂OC(═O)R⁷ wherein R⁷ is C₁-C₈ alkyl, or wherein R⁵ and R⁶ are connected to form a ring, to a mammal in need thereof.

16. (canceled)

17. A compound of Formula (II) or a pharmaceutically acceptable salt thereof: 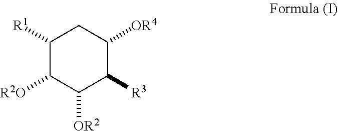
wherein
R¹ is optionally substituted C₁-C₁₀ alkyl, optionally substituted C₁-C₁₀ alkenyl, or optionally substituted C₁-C₁₀ alkynyl;
R² is H or —C(═O)(C₁-C₆)alkyl;
R³ is halo, OH, or O—C(═O)(C₁-C₆)alkyl; and
R⁴ is H, —C(═O)(C₁-C₆)alkyl, or —P(═O)(OR⁵)(OR⁶)], wherein R⁵ and R⁶ are each independently H, (C₁-C₆)alkyl, (CH₂)₂SC(═O)CH₃, CH₂OC(═O)OR⁷ or CH₂OC(═O)R⁷ wherein R7 is C1-C8 alkyl, or wherein R⁵ and R⁶ are connected to form a ring,
wherein when R¹ is CH₃, R² is not H, R³ is not OH, and R⁴ is not H.

18. A composition comprising the compound of claim 17.

19. The composition of claim 18 further comprising a pharmaceutically acceptable carrier.

20. The method of claim 15 wherein R¹ is CH₃ or CHCH₂, R² is H, R³ is OH, and R⁴ is H or —P(═O)(OR⁵)(OR⁶), wherein R⁵ and R⁶ are H.

21. The method of claim 15 wherein the compound is [chemical structure]
